(19) United States
(12) Patent Application Publication
(10) Pub. No.: US 2021/0386858 A1
(43) Pub. Date: Dec. 16, 2021

(54) MUCOADHESIVE COMPOSITIONS AND USES THEREOF

(71) Applicant: BLUEPHARMA - INDUSTRIA FARMACÊUTICA, S.A., Coimbra (PT)

(72) Inventors: Francisca Bastos Silva, Coimbra (PT); António Lucas Nunes, Coimbra (PT); Cláudia Sousa Silva, Coimbra (PT); Sérgio Paulo Simões, Coimbra (PT)

(73) Assignee: BLUEPHARMA - INDUSTRIA FARMACÊUTICA, S.A., Coimbra (PT)

(21) Appl. No.: 17/431,941

(22) PCT Filed: Feb. 18, 2020
(86) PCT No.: PCT/PT2020/050009

§ 371 Date: Aug. 18, 2021

Related U.S. Application Data
(60) Provisional application No. 62/807,680, filed on Feb. 19, 2019.

(51) Int. Cl. A61K 47/32; A61K 47/10; A61K 47/38

(52) U.S. Cl. CPC A61K 47/32; A61K 47/38; A61K 47/10

(57) ABSTRACT

Provided herein are compositions, which may be useful for mucosal delivery of active agents. The provided compositions may be useful for treating diseases.

### BACKGROUND

Oral drug delivery is a common route of drug administration. Alternative routes of drug administration may help to overcome some of the limitations associated with the oral route improving treatments safety and/or efficacy. One such example is the mucosal route of drug delivery (e.g., otic, ophthalmic, oral, oropharyngeal, urethral, vaginal, rectal, nasal), which may offer several advantages.

Mucosal compositions may be developed to deliver drugs locally or to ensure a systemic drug distribution. The mucosal drug administration has the potential to improve drug bioavailability, when compared with the oral route, due to a reduction of the hepatic first pass metabolism and pre-systemic degradation. Mucosal delivery systems are also developed to provide a faster onset of action of the drug substances that is desirable in emergency treatments. The mucosal drug administration is easy and convenient and does not need water for ingestion, which may be particularly relevant for pediatric and geriatric patients.

Some publications disclosed a film-forming composition for mucosal application. For example, U.S. Pat. No. 5,081,158 (the '158 patent) disclose a film-forming composition supported by an esterification reaction of weak organic acids. In particular, the '158 patent relies on tannic acid and salicylic acid to esterify the polymer, and boric acid to aid in polymer crosslinking. The major drawback is that the presence of residual levels of free organic acids that do not participate in the esterification or crosslinking reactions may cause local irritation. The compositions disclosed in the '158 patent are mainly applied in localized and dried regions. The '158 patent discloses that it is recommended to remove as much of the water, moisture, or other body fluids from the surface of the body tissue before applying the composition, limiting its applicability.

WO2014140475 (the '475 publication) discloses topical film-forming compositions for the topical treatment of areas subject to movement, tensions, or elongations, particularly the face or joints. The '475 publication discloses that the solid protective film is formed following solvent evaporation. However, the '475 publication does not disclose if the compositions are mucoadhesive once applied to a wet or moist tissue. Moreover, the '475 publication only discloses the administration of active compounds locally, and not systemically.

WO2007031753 (the '753 publication) discloses film-forming compositions that can be administered either locally to the skin, or into the systemic circulation after passage through the skin. The '753 publication mainly rely on the use of highly volatile solvent mixtures with film-forming and propellant agents. Besides the use of highly volatile solvents that are often not pharmaceutically acceptable for mucosal administration, the compositions in the '753 publication also include a propellant for the formulation delivery from the spray device. In particular, the advantage presented by the '753 publication lies in the combined high saturation levels of the drug substance and the use of the propellant. The propellant will aid in the development of pressure within the container, supplying the necessary force to expel the formulation when the valve is opened. The '753 publication discloses that, during this process, an explosive decompression of the propellant causes the disruption and loss of solvent by evaporation. Propellant-based products may have other disadvantages. For example, their repeated use could irritate the skin, and the prolonged use can pose safety concerns.

Therefore, in order to develop an effective and safe mucoadhesive drug delivery system, it may be important to obtain a pharmaceutical acceptable composition that may form a film to cover the mucosal surface and promote adhesion to the mucosal surface.

Major challenges of mucosal compositions may often be associated to the complexity of maintaining the compositions in direct contact under wet conditions, such as the ones existing on a mucosal surface, for a long time. Therefore, there is a need to develop effective mucoadhesive compositions, able to prolong the contact between the pharmaceutical acceptable formulation and the mucosal surface. Preferably, mucosal compositions should adhere to the surface of the mucosa long enough to allow the release of the active agents and their permeation into the bloodstream. Mucosal compositions should have versatility to incorporate active agents very different in nature (e.g. pharmaceutical agents with different physicochemical properties, nutraceutical agents, dietary supplements, or cosmetic agents), and a high active agent loading capacity, while maintaining stability and resistance to temperature and relative humidity without compromising performance, adhesion, and appearance.

### BRIEF SUMMARY OF THE INVENTION

The present invention (disclosure) relates to compositions (e.g., pharmaceutical compositions, nutraceutical compositions, cosmetic compositions, supplementary compositions), which may be useful for mucosal delivery of active agents (e.g., pharmaceutical agent, nutraceutical agent, cosmetic agent, supplement). In certain embodiments, the composition is a solution, gel, suspension, or emulsion, under ambient conditions. Also provided herein are kits containing the compositions and instructions for use. Further provided herein are use of the compositions for delivering an active agent, treating a disease, and/or preventing a disease.

In certain embodiments, the composition comprises: (i) a polyacrylic acid, wherein the concentration of the polyacrylic acid in the composition is between 0.1% and 10%, inclusive, by weight; (ii) a polyethylene glycol (PEG), wherein the concentration of the PEG in the composition is between 0% and 90%, inclusive, by weight; (iii) ethanol, a propanol, a butanol, or a mixture thereof, wherein the concentration of component (iii) in the composition is between 0% and 60%, inclusive, by weight; and (iv) water, wherein the concentration of water in the composition is between 10% and 75%, inclusive, by weight; provided that the total concentration of all components of the composition is 100%, inclusive, by weight.

In some embodiments, the composition comprises: (i) a polyvinylpyrrolidone copolymer, wherein the concentration of the polyvinylpyrrolidone copolymer in the composition is between 0.1% and 25%, inclusive, by weight; (ii) a polyethylene glycol (PEG), wherein the concentration of the PEG in the composition is between 1% and 90%, inclusive, by weight; (iii) ethanol, a propanol, a butanol, or a mixture thereof, wherein the concentration of component (iii) in the composition is between 1% and 50%, inclusive, by weight; and (iv) water, wherein the concentration of water in the composition is between 5% and 75%, inclusive, by weight; provided that the total concentration of all components of the composition is 100%, inclusive, by weight.

In certain embodiments, the composition comprises: (i) one or both of a hydroxypropyl cellulose M and a hydroxypropyl cellulose G, wherein: when the composition comprises a hydroxypropyl cellulose M, the concentration of the hydroxypropyl cellulose M in the composition is between 0.1% and 5%, inclusive, by weight; when the composition comprises a hydroxypropyl cellulose G, the concentration of the hydroxypropyl cellulose G in the composition is between 0.1% and 7%, inclusive, by weight; and when the composition comprises only one or both of a hydroxypropyl cellulose M and a hydroxypropyl cellulose G, the composition further comprises a polyvinylpyrrolidone copolymer, wherein the concentration of the polyvinylpyrrolidone copolymer in the composition is between 0% and 10%, inclusive, by weight; (ii) ethanol, a propanol, a butanol, or a mixture thereof, wherein the concentration of component (ii) thereof in the composition is between 10% and 80%, inclusive, by weight; and (iii) water, wherein the concentration of water in the composition is between 8% and 75%, inclusive, by weight; provided that the total concentration of all components of the composition is 100%, inclusive, by weight.

In certain embodiments, the composition further comprising an active agent, wherein the concentration of the active agent in the composition is between 0.1% and 50%, inclusive, by weight.

The disclosure further provides kits comprising a composition and instructions for using the composition.

The disclosure further provides methods of delivering an active agent to a subject in need thereof comprising contacting a mucus of the subject in need thereof with the composition.

The disclosure further provides methods of treating a disease in a subject in need thereof comprising contacting a mucus of the subject in need thereof with an effective amount of the composition.

The disclosure further provides methods of preventing a disease in a subject in need thereof comprising contacting a mucus of the subject in need thereof with an effective amount of the composition.

In certain embodiments, the subject is a human. In certain embodiments, the subject is an animal.

The details of one or more embodiments of the present disclosure are set forth herein. Other features, objects, and advantages of the present disclosure will be apparent from the Detailed Description, Examples, and Claims.

### DETAILED DESCRIPTION OF CERTAIN EMBODIMENTS OF THE INVENTION

The present invention relates to compositions, which may be useful for mucosal delivery of active agents. Also provided herein are kits containing the compositions and instructions for use. Further provided herein are use of the compositions for delivering an active agent, treating a disease, and/or preventing a disease.

### Pharmaceutical Compositions, Kits, and Administration

In one aspect, the present disclosure provides compositions, which may be useful for mucosal delivery of active agents to a subject (e.g., a human or animal), biological sample, or cell. The mucus includes otic, ophthalmic, oral, oropharyngeal, urethral, vaginal, rectal, and nasal mucosa. In certain embodiments, the mucus is oral mucus. The compositions may be useful in delivering active agents via a mucosal membrane into the bloodstream. Compared to compositions for other delivery routes, e.g., compositions for oral delivery, the compositions described herein may be able to reduce pre-systemic degradation, reduce hepatic first-pass metabolism, increase bioavailability, reduce active agent dosages, increase safety, provide a faster onset of action of the active agent, reduce intrusion to the subject, and/or reduce complications related to blood extravasation and infection. In certain embodiments, the composition does not require medical support, easy to use, and does not need water for ingestion, which may be particularly relevant for pediatric and geriatric subjects. The composition may increase compliance and convenience of the subject, e.g., during medium- and long-term use of the composition.

The mucosal membranes, depending on their functionality, may have a particular cellular physiology and may depict different physiological obstacles, such as available surface area for absorption, permeability rate, local pH, texture of the membrane surface, clearance mechanism, and presence of proteolytic enzymes in and/or around the mucosa. These aspects along with the physicochemical properties of the active agent to be delivered and the local or systemic effect desired may need to be taken into consideration throughout the development of the mucosal compositions/delivery systems. Some challenges of mucosal delivery arise from the nature of the mucosal tissues that are generally glabrous and wet, which may interfere with attempts to retain the composition at and/or on these tissues. The physiological events, such as the clearance mechanism, musculature activity, and/or “washing effect” of mucosal fluids may also compromise the retention time of the compositions at the site of local application, action, and/or absorption. It may be desired to maintain the composition in direct contact with the mucus for a sufficiently long time for effective delivery.

The compositions described herein may strengthen the interaction and adhesion of the composition with the mucus and mucosal surfaces, may enhance mucoadhesion, may maintain the composition in direct contact with mucosal surface, and may potentiate the delivery of active agents at, on and/or into the mucus. The compositions may provide superior film forming ability and film stability, clean and better mucosal feel, and/or higher active agent loading.

The compositions described herein may promote the coating of mucosal surfaces. In certain embodiments, the compositions are substantially free of a propellant. In certain embodiments, the term “substantially free” refers to at least 90%, at least 95%, at least 97%, at least 99%, or at least 99.9% free. In certain embodiments, the composition has film-forming capacity and modulated viscosity. In certain embodiments, the composition is a liquid (e.g., solution, suspension, emulsion). In certain embodiments, the composition is a gel. In certain embodiments, the composition is administered as a spray. In certain embodiments, the composition is in the form of a spray. In certain embodiments, the composition is administered as drops. In certain embodiments, the composition is in the form of drops.

The terms “mucosal membrane” and “mucosal surface” are used interchangeably.

The terms “composition” and “formulation” are used interchangeably.

“Mucoadhesion” as used herein refers to the feature of a composition that promotes adhesion once in contact with a mucosa or mucosal-like surfaces.

“Film forming capacity” as used herein refers to the ability of a composition to cover and form a layer on the surface of the mucosal membrane for example, after a solvent accompanying the film former has evaporated, absorbed into and/or dissipated on the substrate.

“Modulated of viscosity” as used herein refers to the capacity to adjust the viscosity of the composition, in particular envisioning the dispensing approach.

“Liquid-based formulations” can be distinguished from “Gel-based formulations” on the basis of viscosity. “Gel-based formulations” are generally more viscous than “liquid-based formulations”. In the context of this invention, the viscosity of “liquid-based formulations” is below 15,000 mPas, and allows the dispensing using, but not limiting to a spray-pump device or dropper.

It is an object of certain embodiment of the present invention to provide a formulation (e.g., liquid formulation) to be dispensed through a metered dose device, either a spray-pump or a dropper, and a composition that is able to promote adhesion with the mucus or mucosal-like surfaces at a versatile range of pHs, ranging from 1 to 14.

It is an object of certain embodiment of the present invention to provide a composition able to incorporate active agents.

In the context of this invention, active agents refers to natural or synthetic compound(s) capable of activity or other direct effect in the diagnosis, cure, mitigation, treatment or prevention of disease or to affect the structure and function of the body. In any of the foregoing embodiments, a provided mucoadhesive composition may contain one or more active agents, including, but not limiting to pharmaceutical agent that belong to different Biopharmaceutics Classification System (BCS), for example from BCS class I, II, III or IV, and/or peptides, and/or vaccines and/or nucleic acid-based products and/or immunologic agents and/or phytopharmaceutical agents and/or nutraceutical agents and/or cosmetic agents and/or supplements. In certain embodiments, the active agent is a small molecule (e.g., when the molecular weight is lower than 500, 800, 1000, or 1500, g/mol). In certain embodiments, the active agent is a peptide or protein (e.g., peptide/protein that comprises 3-10, 10-100, 100-1,000, 1,000-10,000, or 10,000-100,000, inclusive, amino acids). In certain embodiments, the active agent is a drug approved by the U.S. Food and Drug Administration and/or the European Medicines Agency.

It is an object of certain embodiment of the present invention to provide a composition containing active agent(s) which can be administered in a manner that will cause substantial systemic absorption without substantial risk of the dose passing into the lungs of the recipient.

One or more of the above-mentioned objects and others are achieved by the present invention. In certain embodiments, the compositions of the present disclosure that include one or more active agents are administered sublingually or buccally, in which a substantial portion of the active agent will not be passed into the lungs of the patient.

In certain embodiments, a provided composition or the combination of the excipients included in a provided composition has one or more of the following attributes: it is pharmaceutically acceptable; it is not irritant for the mucosa; it encompasses a substantial degree of flexibility in order to promote the desired entanglement with mucus; it has an appropriate molecular weight/length to allow chain inter-penetration; it has charges; it has functional groups to form hydrogen bonds; it has adequate surface energy to promote a good wetting and spreadability; it is tasteless; it is readily available; it is inexpensive; it does not decompose during retention time; it allows easy incorporation of the bioactive substances and provide the release of the active agents in a controlled manner; it promotes local and/or systemic absorption; it provides mucoadhesion; it promotes film-formation; it demonstrates local enzyme inhibition and penetration enhancement properties and/or it dissolves on the mucosal milieu.

In the present invention, one or a mixture of different excipients (e.g., pharmaceutically acceptable excipients) are combined to achieve an adequate viscosity, the target film-forming capability, and an appropriate mucoadhesiveness. In the embodiment of the present invention, one or a mixture of different polymers, plasticizers, organic solvents, inorganic solvents, achieves the adequate viscosity, film-forming capability and mucoadhesiveness of the composition. In the embodiment of the present invention, the adequate viscosity, film-forming capability and mucoadhesiveness of the composition is achieved by one or mixture of different grades of polyacrylic acid, polyvinylpyrrolidone copolymer, polyvidone, povidone, hydroxypropyl cellulose, other cellulose derivatives, polyethylene glycol, organic solvent and/or inorganic solvent. In certain embodiment of the present invention, the adequate viscosity, film-forming capability and mucoadhesiveness of the composition is achieved by one or mixture of Carbopol®, with concentrations from about 0.1% to about 10%; polyethylene glycol (PEG) with concentrations from about 0% to about 90%; ethanol with concentrations from about 0% to about 60% and water with concentration from about 10% to about 75%; Provided that the total concentration of all components of the liquid formulation is 100%, inclusive, by weight. In certain embodiment of the present invention, the adequate viscosity, the film-forming capability and mucoadhesiveness of the composition is achieved by one or mixture of polyvinylpyrrolidone copolymer, with concentrations from about 0.1% to about 25%; polyethylene glycol (PEG) with concentrations from about 1% to about 90%; Ethanol with concentrations from about 1% to about 50% and water with concentration from about 5% to about 75%; Provided that the total concentration of all components of the liquid formulation is 100%, inclusive, by weight. In certain embodiment of the present invention, the adequate viscosity, the film-forming capability and mucoadhesiveness of the composition is achieved by one or mixture of hydroxypropyl cellulose M (HPC-M) and a hydroxypropyl cellulose G (HPC-G) with concentrations from about 0.1% to about 5% and from about 0.1% to about 7% respectively; polyvinylpyrrolidone copolymer with concentrations from about 0% to about 10%; Ethanol with concentrations from about 10% to about 80% and Water with concentration from about 8% to about 75%; Provided that the total concentration of all components of the liquid formulation is 100%, inclusive, by weight.

In one aspect, the disclosure provides compositions comprising: (i) a polyacrylic acid, wherein the concentration of the polyacrylic acid in the composition is between 0.1% and 10%, inclusive, by weight; (ii) a polyethylene glycol (PEG), wherein the concentration of the PEG in the composition is between 0% and 90%, inclusive, by weight; (iii) ethanol, a propanol, a butanol, or a mixture thereof, wherein the concentration of component (iii) in the composition is between 0% and 60%, inclusive, by weight; and (iv) water, wherein the concentration of water in the composition is between 10% and 75%, inclusive, by weight; provided that the total concentration of all components of the composition is 100%, inclusive, by weight. In certain embodiments, the composition further comprises a cellulose derivative, a polyol, and/or a cyclodextrin. In some embodiments, the composition further comprises a pH adjuster, a sweetener, a colorant, and/or a flavoring agent.

In some embodiments, the composition contains a polyacrylic acid. In certain embodiments, the polyacrylic acid in the composition is a Carbomer® polymer. In certain embodiments, the Carbomer® polymer is selected from the group consisting of 910, 934, 940, 941, 934P, and 962. In some embodiments, the polyacrylic acid is a Carbopol® polymer. In certain embodiments, the Carbopol® polymer is selected from the group consisting of 71G NF, 971P NF, 974P NF, 980 NF, 981 NF, 5984 EP, ETD 2020 NF, Ultrez 10 NF, 934 NF, 934P NF, 940 NF, 941 NF, 1342 NF. In certain embodiments, the Carbopol® polymer is selected from the group consisting of 71G NF, 971P NF, 974P NF, and 934P NF. In certain embodiments, the Carbopol® polymer is selected from the group consisting of 971P NF, 974P NF, ETD 2020 NF, 980 NF, and 956. In certain embodiments, the polyacrylic acid is Carbopol® 971P NF polymer.

In certain embodiments, the concentration of the polyacrylic acid in the composition is between 0.1% and 10%, inclusive, by weight. In some embodiments, the concentration of the polyacrylic acid in the composition is between 0.1% and 9%, inclusive, by weight. In certain embodiments, the concentration of the polyacrylic acid in the composition is between 0.1% and 3%, inclusive, by weight. In some embodiments, the concentration of the polyacrylic acid in the composition is between 0.2% and 3%, inclusive, by weight. In some embodiments, the concentration of the polyacrylic acid in the composition is between 0.1% and 1%, inclusive, by weight. In certain embodiments, the concentration of the polyacrylic acid in the composition is between 0.2% and 0.9%, inclusive, by weight.

In some embodiments, the composition further comprises a PEG. In some embodiments, the PEG in the composition is a PEG between PEG200 and PEG600, inclusive. In certain embodiments, the PEG is selected from the group consisting of PEG200, PEG250, PEG300, PEG350, PEG400, PEG450, PEG500, PEG550, and PEG600. In some embodiments, the PEG is PEG400.

In some embodiments, the concentration of the PEG in the composition is 0% by weight. In some embodiments, the concentration of the PEG in the composition is between 0% and 90%, inclusive, by weight. In some embodiments, the concentration of the PEG in the composition is between 0.1% and 90%, inclusive, by weight. In some embodiments, the concentration of the PEG in the composition is between 0.1% and 80%, inclusive, by weight. In some embodiments, the concentration of the PEG in the composition is between 0% and 76%, inclusive, by weight. In some embodiments, the concentration of the PEG in the composition is between 0.75% and 76%, inclusive, by weight. In some embodiments, the concentration of the PEG in the composition is between 1.5% and 76%, inclusive, by weight. In certain embodiments, the concentration of the PEG in the composition is between 1.5% and 65%, inclusive, by weight. In certain embodiments, the concentration of the PEG in the composition is between 1.5% and 40%, inclusive, by weight.

In certain embodiments, component (iii) of the composition is ethanol. In certain embodiments, component (iii) of the composition is a propanol (e.g., 1-propanol, 2-propanol). In certain embodiments, component (iii) of the composition is a butanol (e.g., 1-butanol, 2-butanol, isobutanol, tert-butanol). In certain embodiments, component (iii) is a mixture of ethanol and a propanol. In certain embodiments, component (iii) is a mixture of ethanol and a butanol. In certain embodiments, component (iii) is a mixture of a propanol and a butanol. In certain embodiments, component (iii) is a 1:1 mixture of ethanol and a propanol. In certain embodiments, component (iii) is a 1:1 mixture of ethanol and a butanol. In certain embodiments, component (iii) is a 1:1 mixture of a propanol and a butanol.

In some embodiments, the concentration of component (iii) in the composition is 0% by weight. In some embodiments, the concentration of component (iii) in the composition is between 0% and 60%, inclusive, by weight. In some embodiments, the concentration of component (iii) in the composition is between 0% and 40%, inclusive, by weight. In some embodiments, the concentration of component (iii) in the composition is between 4% and 60%, inclusive, by weight. In certain embodiments, the concentration of component (iii) in the composition is between 5% and 40%, inclusive, by weight. In certain embodiments, the concentration of component (iii) in the composition is between 10% and 40%, inclusive, by weight. In some embodiments, the concentration of component (iii) in the composition is between 12% and 40%, inclusive, by weight. In some embodiments, the concentration of component (iii) in the composition is between 12% and 35%, inclusive, by weight. In certain embodiments, the concentration of component (iii) in the composition is between 15% and 35%, inclusive, by weight.

In some embodiments, the concentration of water in the composition is between 10% and 75%, inclusive, by weight. In some embodiments, the concentration of water in the composition is between 10.5% and 63%, inclusive, by weight. In some embodiments, the concentration of water in the composition is between 10.5% and 58%, inclusive, by weight. In some embodiments, the concentration of water in the composition is between 10% and 51%, inclusive, by weight. In certain embodiments, the concentration of water in the composition is between 40% and 57%, inclusive, by weight.

In certain embodiments, the composition further comprises a cellulose derivative. In some embodiments, the cellulose derivative is selected from the group consisting of methylcellulose, carboxymethyl cellulose, ethyl cellulose, ethyl methyl cellulose, hydroxypropyl cellulose, and hydroxypropyl methylcellulose. In certain embodiments, the cellulose derivative is methylcellulose. In certain embodiments, the cellulose derivative is hydroxypropyl cellulose. In certain embodiments, the cellulose derivative is methylcellulose and hydroxypropyl cellulose.

In certain embodiments, the concentration of the cellulose derivative in the composition is between 0% to 10%, inclusive, by weight. In certain embodiments, the concentration of the cellulose derivative in the composition is between 0.01% to 10%, inclusive, by weight. In certain embodiments, the concentration of the cellulose derivative in the composition is between 0.03% to 3%, inclusive, by weight. In some embodiments, the concentration of the cellulose derivative in the composition is between 0.3% and 3%, inclusive, by weight. In some embodiments, the concentration of the cellulose derivative in the composition is between 0% and 1.8%, inclusive, by weight. In certain embodiments, the concentration of the cellulose derivative in the composition is between 0.03% and 1.8%, inclusive, by weight. In certain embodiments, the concentration of the cellulose derivative in the composition is between 0.3% and 1.8%, inclusive, by weight. In some embodiments, the concentration of the cellulose derivative in the composition is between 0.8% and 1.8%, inclusive, by weight. In certain embodiments, the concentration of the cellulose derivative in the composition is between 1% and 1.8%, inclusive, by weight. In some embodiments, the concentration of the cellulose derivative in the composition is between 1% and 1.5%, inclusive, by weight.

In certain embodiments, the composition is substantially free of cellulose derivatives.

In certain embodiments, the composition further comprises a polyol. In some embodiments, the polyol is selected from the group of glycerol, erythritol, isomalt, maltitol, sorbitol, lactitol, mannitol, xylitol, trimethylolpropane, and pentaerythritol. In certain embodiments, the polyol is glycerol.

In some embodiments, the composition further comprises a polyol wherein the concentration of the polyol is between 0% to 5%, inclusive, by weight. In some embodiments, the concentration of the polyol in the composition is between 0.001% and 5%, inclusive, by weight. In some embodiments, the concentration of the polyol in the composition is between 0% and 3%, inclusive, by weight. In some embodiments, the concentration of the polyol in the composition is between 0.001% and 3%, inclusive, by weight. In some embodiments, the concentration of the polyol in the composition is between 0.8% and 3%, inclusive, by weight. In some embodiments, the concentration of the polyol in the composition is between 0.9% and 3%, inclusive, by weight.

In certain embodiments, the composition further comprises a cyclodextrin. In some embodiments, the cyclodextrin is selected from the group consisting of alpha-cyclodextrin, beta-cyclodextrin, gamma-cyclodextrin, methyl-beta-cyclodextrin, hydroxypropyl-beta-cyclodextrin, and hydroxypropyl-gamma-cyclodextrin. In some embodiments, the cyclodextrin is selected from the group consisting of Cavasol® W7 HP, Cavasol® W6 HP, Cavasol® W8 HP, Cavasol® W7 M, Cavitron® W7 HP5, Cavitron® W7 HP7, Cavamax® W8 Cavamax® W6, and Cavamax® W7.

In some embodiments, the concentration of cyclodextrin in the composition is between 0 to 30%, inclusive, by weight. In some embodiments, the concentration of cyclodextrin in the composition is between 0.1 to 30%, inclusive, by weight. In some embodiments, the concentration of the cyclodextrin in the composition is between 0.3% and 29%, inclusive, by weight. In some embodiments, the concentration of the cyclodextrin in the composition is between 3% and 9%, inclusive, by weight. In some embodiments, the concentration of the cyclodextrin in the composition is between 25% and 30%, inclusive, by weight. In certain embodiments, the cyclodextrin increases the solubility of the active agent in the composition. In certain embodiments, when the composition further comprises a cyclodextrin, the composition is substantially free of component (iii).

In certain embodiments, a provided composition further comprises a sweetener. In some embodiments, the concentration of the sweetener in the composition is between 0.01% and 5%, inclusive, by weight. In some embodiments, the concentration of the sweetener in the composition is between 0.01% and 1%, inclusive, by weight. In some embodiments, the concentration of the sweetener in the composition is between 0.01% and 0.03%, inclusive, by weight.

In certain embodiments, a provided composition further comprises a pH adjuster. In certain embodiments, the pH adjuster is an inorganic acid. In some embodiments, the pH adjuster is an inorganic base (e.g., alkali metal hydroxide). In certain embodiments, the pH adjuster is an organic acid. In some embodiments, the pH adjuster is an organic base. In some embodiments, the pH adjuster is NaOH. In certain embodiments, the pH adjuster is HCl. In some embodiments, the concentration of the pH adjuster in the composition is between 0.007% and 5%, inclusive, by weight. In some embodiments, the concentration of the pH adjuster in the composition is between 0.007% and 2.5%, inclusive, by weight. In some embodiments, the concentration of the pH adjuster in the composition is between 0.007% and 1.7%, inclusive, by weight. In some embodiments, the concentration of the pH adjuster in the composition is between 0.007% and 0.12%, inclusive, by weight.

In certain embodiments, a composition further comprises a colorant. In some embodiments, the concentration of the colorant in the composition is between 0.001% and 5%, inclusive, by weight. In some embodiments, the concentration of the colorant in the composition is between 0.001% and 2.5%, inclusive, by weight. In some embodiments, the concentration of the colorant in the composition is between 0.1% and 1%, inclusive, by weight.

In certain embodiments, a provided composition further comprises a flavoring agent (flavor). In some embodiments, the concentration of the flavoring agent in the composition is between 0.001% and 5%, inclusive, by weight. In some embodiments, the concentration of the flavoring agent in the composition is between 0.001% and 2.5%, inclusive, by weight. In some embodiments, the concentration of the flavoring agent in the composition is between 0.1% and 1%, inclusive, by weight.

In certain embodiments, the pH value of the composition under ambient conditions is between 3.5 and 9, inclusive. In certain embodiments, the pH value of the composition under ambient conditions is between 4.5 and 8, inclusive. In certain embodiments, the pH value of the composition under ambient conditions is between 5.5 and 7, inclusive.

In certain embodiments, the composition is as described in Tables 1A, 1B, and 5.

In certain embodiments, the composition comprises:

(i) a polyacrylic acid (e.g., Carbopol), wherein the concentration of the polyacrylic acid in the composition is 0.2-3%, inclusive, by weight;

(ii) a polyethylene glycol (PEG) (e.g., PEG400), wherein the concentration of the PEG in the composition is 0-76%, inclusive, by weight;

(iii) ethanol, wherein the concentration of ethanol in the composition is 0-40%, inclusive, by weight; and

(iv) water, wherein the concentration of water in the composition is 10.5-63%, inclusive, by weight.

In certain embodiments, the composition described in this paragraph further comprises:

a first cellulose derivative (e.g., HPC), wherein the concentration of the first cellulose derivative in the composition is 0-1.8%, inclusive, by weight; a second cellulose derivative (e.g., MC), wherein the concentration of the second cellulose derivative in the composition is 0-3%, inclusive, by weight; provided that the combined concentration of the first and second first cellulose derivatives is 0-3%;

a polyol (e.g., glycerol), wherein the concentration of the polyol in the composition is 0-5%, inclusive, by weight; and

a cyclodextrin, wherein the concentration of the cyclodextrin in the composition is 0-30%, inclusive, by weight.

In certain embodiments, the composition comprises:

(i) a polyacrylic acid (e.g., Carbopol), wherein the concentration of the polyacrylic acid in the composition is 0.2-0.9%, inclusive, by weight;

(ii) a polyethylene glycol (PEG) (e.g., PEG400), wherein the concentration of the PEG in the composition is 0.75-76%, inclusive, by weight;

(iii) ethanol, wherein the concentration of ethanol in the composition is 12-40%, inclusive, by weight; and

(iv) water, wherein the concentration of water in the composition is 10.5-63%, inclusive, by weight.

In certain embodiments, the composition described in this paragraph further comprises:

a first cellulose derivative (e.g., HPC), wherein the concentration of the first cellulose derivative in the composition is 0-1.8%, inclusive, by weight; a second cellulose derivative (e.g., MC), wherein the concentration of the second cellulose derivative in the composition is 0-1.8%, inclusive, by weight; provided that the combined concentration of the first and second first cellulose derivatives is 0-1.8%;

a polyol (e.g., glycerol), wherein the concentration of the polyol in the composition is 0-3%, inclusive, by weight; and

a cyclodextrin, wherein the concentration of the cyclodextrin in the composition is 0-30%, inclusive, by weight.

In certain embodiments, the composition comprises:

(i) a polyacrylic acid (e.g., Carbopol), wherein the concentration of the polyacrylic acid in the composition is 0.2-0.9%, inclusive, by weight;

(ii) a polyethylene glycol (PEG) (e.g., PEG400), wherein the concentration of the PEG in the composition is 1.5-76%, inclusive, by weight;

(iii) ethanol, wherein the concentration of ethanol in the composition is 12-35%, inclusive, by weight; and

(iv) water, wherein the concentration of water in the composition is 10.5-58%, inclusive, by weight.

In certain embodiments, the composition described in this paragraph further comprises:

a first cellulose derivative (e.g., HPC), wherein the concentration of the first cellulose derivative in the composition is 0-1.8%, inclusive, by weight; a second cellulose derivative (e.g., MC), wherein the concentration of the second cellulose derivative in the composition is 0.03-1.8%, inclusive, by weight; provided that the combined concentration of the first and second first cellulose derivatives is 0.3-1.8%; and

a polyol (e.g., glycerol), wherein the concentration of the polyol in the composition is 0.9-3%, inclusive, by weight.

In certain embodiments, the composition comprises:

(i) a polyacrylic acid (e.g., Carbopol), wherein the concentration of the polyacrylic acid in the composition is about 0.3%, by weight;

(ii) a polyethylene glycol (PEG) (e.g., PEG400), wherein the concentration of the PEG in the composition is about 7.92%, by weight;

(iii) ethanol, wherein the concentration of ethanol in the composition is about 33%, by weight; and

(iv) water, wherein the concentration of water in the composition is about 57%, by weight.

In certain embodiments, the composition described in this paragraph further comprises a cellulose derivative (e.g., MC), wherein the concentration of the cellulose derivative in the composition is about 1.78%, by weight.

The term “about XX,” wherein XX is a number or percentage, refers to a range. In certain embodiments, “about XX” refers to XX±5%. In certain embodiments, “about XX” refers to XX±10%. In certain embodiments, “about XX” refers to XX±20%. In certain embodiments, “about XX” refers to XX±40%. For example, when “about XX” refers to XX±10%, “about 0.3%” refers to a range between 0.3%×(100%−10%) and 0.3%×(100%+10%), i.e., 0.27-0.33%, inclusive.

In another aspect, the disclosure provides compositions comprising: (i) a polyvinylpyrrolidone copolymer, wherein the concentration of the polyvinylpyrrolidone copolymer in the composition is between 0.1% and 25%, inclusive, by weight; (ii) a polyethylene glycol (PEG), wherein the concentration of the PEG in the composition is between 1% and 90%, inclusive, by weight; (iii) ethanol, a propanol, a butanol, or a mixture thereof, wherein the concentration of component (iii) in the composition is between 1% and 50%, inclusive, by weight; and (iv) water, wherein the concentration of water in the composition is between 5% and 75%, inclusive, by weight; provided that the total concentration of all components of the composition is 100%, inclusive, by weight. The composition may further comprise a cellulose derivate, a cyclodextrin, a polyol, and/or a polyvinylpyrrolidone homopolymer. In some embodiments, the composition further comprises a pH adjuster, a pH adjustment agent, a sweetener, a colorant, and/or a flavoring agent.

In some embodiments, the composition further comprises a polyvinylpyrrolidone copolymer. In some embodiments, the polyvinylpyrrolidone copolymer is poly(vinylpyrrolidone-co-vinyl acetate) 64. In certain embodiments, the polyvinylpyrrolidone copolymer is poly(vinylpyrrolidone-co-vinyl acetate) 64. In certain embodiments, the polyvinylpyrrolidone copolymer is select from the group of PVP/VA E-335, PVP/VA E-535, PVP/VA E-635, PVP/VA E-735, PVP/VA 1-335, PVP/VA 1-735, PVP/VA S-630, PVP/VA S-630L, PVP/VA W-535, PVP/VA W-635, PVP/VA W-735, Luvitec® VA 64 W, Luvitec® VA 64 P, Luvitec® VPC 55 K 65 W, Kollidon® VA 64, and Collacral® Val. In certain embodiments, the polyvinylpyrrolidone copolymer is Kollidon® VA 64.

In certain embodiments, the concentration of the polyvinylpyrrolidone copolymer in the composition is between 0.1% and 25%, inclusive, by weight. In some embodiments, the concentration of the polyvinylpyrrolidone copolymer in the composition is between 0.5% and 20%, inclusive, by weight. In some embodiments, the concentration of the polyvinylpyrrolidone copolymer in the composition is between 0.5% and 10%, inclusive, by weight. In some embodiments, the concentration of the polyvinylpyrrolidone copolymer in the composition is between 0.7% and 10%, inclusive, by weight. In certain embodiments, the concentration of the polyvinylpyrrolidone copolymer in the composition is between 5% and 10%, inclusive, by weight. In certain embodiments, the concentration of the polyvinylpyrrolidone copolymer in the composition is between 7% and 11%, inclusive, by weight. In some embodiments, the concentration of the polyvinylpyrrolidone copolymer in the composition is between 7% and 10%, inclusive, by weight.

In some embodiments, the composition further comprises a PEG. In some embodiments, the PEG in the composition is a PEG between PEG200 and PEG600, inclusive. In certain embodiments, the PEG is selected from the group consisting of PEG200, PEG250, PEG300, PEG350, PEG400, PEG450, PEG500, PEG550, and PEG600. In some embodiments, the PEG is PEG400.

In some embodiments, the concentration of the PEG in the composition is between 1% and 90%, inclusive, by weight. In some embodiments, the concentration of the PEG in the composition is between 1.85% and 77%, inclusive, by weight. In some embodiments, the concentration of the PEG in the composition is between 1.85% and 76.5%, inclusive, by weight. In some embodiments, the concentration of the PEG in the composition is between 1.85% and 75%, inclusive, by weight. In some embodiments, the concentration of the PEG in the composition is between 19% and 65%, inclusive, by weight. In certain embodiments, the concentration of the PEG in the composition is between 40% and 65%, inclusive, by weight.

In certain embodiments, component (iii) of the composition is ethanol. In certain embodiments, component (iii) of the composition is a propanol (e.g., 1-propanol, 2-propanol). In certain embodiments, component (iii) of the composition is a butanol (e.g., 1-butanol, 2-butanol, isobutanol, tert-butanol). In certain embodiments, component (iii) is a mixture of ethanol and a propanol. In certain embodiments, component (iii) is a mixture of ethanol and a butanol. In certain embodiments, component (iii) is a mixture of a propanol and a butanol. In certain embodiments, component (iii) is a 1:1 mixture of ethanol and a propanol. In certain embodiments, component (iii) is a 1:1 mixture of ethanol and a butanol. In certain embodiments, component (iii) is a 1:1 mixture of a propanol and a butanol.

In some embodiments, the concentration of component (iii) in the composition is between 1% and 50%, inclusive, by weight. In some embodiments, the concentration of component (iii) in the composition is between 2% and 40%, inclusive, by weight. In some embodiments, the concentration of component (iii) in the composition is between 4.3% and 40%, inclusive, by weight. In certain embodiments, the concentration of component (iii) in the composition is between 4.4% and 39%, inclusive, by weight. In certain embodiments, the concentration of component (iii) in the composition is between 10% and 21%, inclusive, by weight. In some embodiments, the concentration of component (iii) in the composition is between 15% and 20%, inclusive, by weight.

In some embodiments, the concentration of water in the composition is between 5% and 75%, inclusive, by weight. In some embodiments, the concentration of water in the composition is between 20% and 75%, inclusive, by weight. In some embodiments, the concentration of water in the composition is between 8% and 54%, inclusive, by weight. In some embodiments, the concentration of water in the composition is between 10% and 57%, inclusive, by weight. In some embodiments, the concentration of water in the composition is between 10% and 51%, inclusive, by weight. In some embodiments, the concentration of water in the composition is between 10% and 40%, inclusive, by weight. In certain embodiments, the concentration of water in the composition is between 50% and 57%, inclusive, by weight.

In certain embodiments, a provided composition further comprises a polyvinylpyrrolidone homopolymer. In certain embodiments, the polyvinylpyrrolidone homopolymer is polyvinylpyrrolidone K12, polyvinylpyrrolidone K15, polyvinylpyrrolidone K25, polyvinylpyrrolidone K29, polyvinylpyrrolidone K30, polyvinylpyrrolidone K60, polyvinylpyrrolidone K90, or polyvinylpyrrolidone K120. In certain embodiments, the polyvinylpyrrolidone homopolymer is polyvinylpyrrolidone K29. In certain embodiments, the polyvinylpyrrolidone homopolymer is polyvinylpyrrolidone K30.

In some embodiments, the concentration of the polyvinylpyrrolidone homopolymer in the composition is between 0.1 to 10%, inclusive, by weight. In some embodiments, the concentration of the polyvinylpyrrolidone homopolymer in the composition is between 0.1% and 3.5%, inclusive, by weight. In some embodiments, the concentration of the polyvinylpyrrolidone homopolymer in the composition is between 0.1% and 1.8%, inclusive, by weight.

In certain embodiments, the composition further comprises a cellulose derivative. In some embodiments, the cellulose derivative is selected from the group consisting of methylcellulose, carboxymethyl cellulose, ethyl cellulose, ethyl methyl cellulose, hydroxypropyl cellulose, and hydroxypropyl methylcellulose. In certain embodiments, the cellulose derivative is methylcellulose. In certain embodiments, the cellulose derivative is hydroxypropyl cellulose. In certain embodiments, the cellulose derivative is methylcellulose and hydroxypropyl cellulose.

In certain embodiments, the concentration of the cellulose derivative in the composition is between 0% to 10%, inclusive, by weight. In certain embodiments, the concentration of the cellulose derivative in the composition is between 0.01% to 10%, inclusive, by weight. In certain embodiments, the concentration of the cellulose derivative in the composition is between 0.03% to 3%, inclusive, by weight. In some embodiments, the concentration of the cellulose derivative in the composition is between 0 and 1.5%, inclusive, by weight. In some embodiments, the concentration of the cellulose derivative in the composition is between 0.03% and 1.5%, inclusive, by weight. In certain embodiments, the concentration of the cellulose derivative in the composition is between 0.4% and 1.5%, inclusive, by weight. In some embodiments, the concentration of the cellulose derivative in the composition is between 1% and 1.5%, inclusive, by weight.

In certain embodiments, the composition is substantially free of cellulose derivatives.

In certain embodiments, the composition further comprises a cyclodextrin. In some embodiments, the cyclodextrin is selected from the group consisting of alpha-cyclodextrin, beta-cyclodextrin, gamma-cyclodextrin, methyl-beta-cyclodextrin, hydroxypropyl-beta-cyclodextrin, and hydroxypropyl-gamma-cyclodextrin. In some embodiments, the cyclodextrin is selected from the group consisting of Cavasol® W7 HP, Cavasol® W6 HP, Cavasol® W8 HP, Cavasol® W7 M, Cavitron® W7 HP5, Cavitron® W7 HP7, Cavamax® W8 Cavamax® W6, and Cavamax® W7.

In some embodiments, the concentration of cyclodextrin in the composition is between 0.3 to 30%, inclusive, by weight. In some embodiments, the concentration of the cyclodextrin in the composition is between 0.3% and 29%, inclusive, by weight. In some embodiments, the concentration of the cyclodextrin in the composition is between 0.3% and 10%, inclusive, by weight. In certain embodiments, the cyclodextrin increases the solubility of the active agent in the composition.

In certain embodiments, the composition further comprises a polyol. In some embodiments, the polyol is selected from the group of glycerol, erythritol, isomalt, maltitol, sorbitol, lactitol, mannitol, xylitol, trimethylolpropane, and pentaerythritol. In certain embodiments, the polyol is glycerol.

In some embodiments, the composition further comprises a polyol wherein the concentration of the polyol is between 0 to 5%, inclusive, by weight. In some embodiments, the composition further comprises a polyol wherein the concentration of the polyol is between 0.01% to 5%, inclusive, by weight. In some embodiments, the composition further comprises a polyol wherein the concentration of the polyol is between 0 to 3%, inclusive, by weight. In some embodiments, the concentration of the polyol in the composition is between 0.01% and 3%, inclusive, by weight. In some embodiments, the concentration of the polyol in the composition is between 0.05% and 3%, inclusive, by weight. In some embodiments, the concentration of the polyol in the composition is between 1.5% and 3%, inclusive, by weight.

In certain embodiments, a provided composition further comprises a sweetener. In some embodiments, the concentration of the sweetener in the composition is between 0% and 5%, inclusive, by weight. In some embodiments, the concentration of the sweetener in the composition is between 0% and 3%, inclusive, by weight. In some embodiments, the concentration of the sweetener in the composition is between 0.01% and 5%, inclusive, by weight. In some embodiments, the concentration of the sweetener in the composition is between 0.01% and 1%, inclusive, by weight. In some embodiments, the concentration of the sweetener in the composition is between 0.01% and 0.03%, inclusive, by weight.

In certain embodiments, a provided composition further comprises a pH adjuster. In certain embodiments, the pH adjuster is an inorganic acid. In some embodiments, the pH adjuster is an inorganic base (e.g., alkali metal hydroxide). In certain embodiments, the pH adjuster is an organic acid. In some embodiments, the pH adjuster is an organic base. In some embodiments, the pH adjuster is NaOH. In certain embodiments, the pH adjuster is HCl. In some embodiments, the concentration of the pH adjuster in the composition is between 0% and 5%, inclusive, by weight. In some embodiments, the concentration of the pH adjuster in the composition is between 0.007% and 5%, inclusive, by weight. In some embodiments, the concentration of the pH adjuster in the composition is between 0.007% and 2.5%, inclusive, by weight. In some embodiments, the concentration of the pH adjuster in the composition is between 0% and 1.7%, inclusive, by weight. In some embodiments, the concentration of the pH adjuster in the composition is between 0.007% and 1.7%, inclusive, by weight. In some embodiments, the concentration of the pH adjuster in the composition is between 0% and 0.12%, inclusive, by weight. In some embodiments, the concentration of the pH adjuster in the composition is between 0.007% and 0.12%, inclusive, by weight.

In some embodiments, the composition further comprises a pH adjustment agent. In some embodiments, the pH adjustment agent is different from the pH adjuster. In certain embodiments, the pH adjustment agent is an inorganic acid. In some embodiments, the pH adjustment agent is an inorganic base. In certain embodiments, the pH adjustment agent is an organic acid. In some embodiments, the pH adjustment agent is an organic base. In some embodiments, the pH adjustment agent is citric acid. In some embodiments, the pH adjustment agent is salicylic acid. In certain embodiments, the concentration of the pH adjustment agent in the composition is between 0% and 2.5%, inclusive, by weight. In certain embodiments, the concentration of the pH adjustment agent in the composition is between 0.017% and 2.5%, inclusive, by weight. In certain embodiments, the concentration of the pH adjustment agent in the composition is between 0% and 1.7%, inclusive, by weight. In certain embodiments, the concentration of the pH adjustment agent in the composition is between 0.1% and 1.7%, inclusive, by weight. In certain embodiments, the concentration of the pH adjustment agent in the composition is between 0% and 0.2%, inclusive, by weight.

In certain embodiments, a composition further comprises a colorant. In some embodiments, the concentration of the colorant in the composition is between 0.001% and 5%, inclusive, by weight. In some embodiments, the concentration of the colorant in the composition is between 0.001% and 2.5%, inclusive, by weight. In some embodiments, the concentration of the colorant in the composition is between 0.1% and 1%, inclusive, by weight.

In certain embodiments, a provided composition further comprises a flavoring agent. In some embodiments, the concentration of the flavoring agent in the composition is between 0 and 5%, inclusive, by weight. In some embodiments, the concentration of the flavoring agent in the composition is between 0.001% and 5%, inclusive, by weight. n some embodiments, the concentration of the flavoring agent in the composition is between 0% and 2.5%, inclusive, by weight. In some embodiments, the concentration of the flavoring agent in the composition is between 0.001% and 2.5%, inclusive, by weight. In some embodiments, the concentration of the flavoring agent in the composition is between 0.1% and 1%, inclusive, by weight.

In certain embodiments, the pH value of the composition under ambient conditions is between 3.5 and 9, inclusive. In certain embodiments, the pH value of the composition under ambient conditions is between 4.5 and 8, inclusive. In certain embodiments, the pH value of the composition under ambient conditions is between 5.5 and 7, inclusive.

In certain embodiments, the composition is as described in Tables 2A, 2B, and 6.

In certain embodiments, the composition comprises:

(i) a polyvinylpyrrolidone copolymer (e.g., PVP-VA-64), wherein the concentration of the polyvinylpyrrolidone copolymer in the composition is 0.7-10%, inclusive, by weight;

(ii) a polyethylene glycol (PEG) (e.g., PEG400), wherein the concentration of the PEG in the composition is 1.85-77%, inclusive, by weight;

(iii) ethanol, wherein the concentration of ethanol in the composition is 4.3-40%, inclusive, by weight; and

(iv) water, wherein the concentration of water in the composition is 8-54%, inclusive, by weight.

In certain embodiments, the composition described in this paragraph further comprises:

a pH adjustment agent (e.g., citric acid), wherein the concentration of the pH adjustment agent in the composition is 0-1.7%, inclusive, by weight;

a cellulose derivative (e.g., HPC), wherein the concentration of the cellulose derivative in the composition is 0-1.5%, inclusive, by weight;

a polyol (e.g., glycerol), wherein the concentration of the polyol in the composition is 0-3%, inclusive, by weight;

a flavoring agent, wherein the concentration of the flavoring agent in the composition is 0-2.5%, inclusive, by weight; and

a sweetener, wherein the concentration of the sweetener in the composition is 0-0.03%, inclusive, by weight. In certain embodiments, the composition comprises:

(i) a polyvinylpyrrolidone copolymer (e.g., PVP-VA-64), wherein the concentration of the polyvinylpyrrolidone copolymer in the composition is 5-10%, inclusive, by weight;

(ii) a polyethylene glycol (PEG) (e.g., PEG400), wherein the concentration of the PEG in the composition is 1.85-77%, inclusive, by weight;

(iii) ethanol, wherein the concentration of ethanol in the composition is 4.4-39%, inclusive, by weight; and

(iv) water, wherein the concentration of water in the composition is 8-54%, inclusive, by weight.

In certain embodiments, the composition described in this paragraph further comprises:

a pH adjustment agent (e.g., citric acid), wherein the concentration of the pH adjustment agent in the composition is 0-0.12%, inclusive, by weight;

a cellulose derivative (e.g., HPC), wherein the concentration of the cellulose derivative in the composition is 0.4-1.5%, inclusive, by weight;

a polyol (e.g., glycerol), wherein the concentration of the polyol in the composition is 0-3%, inclusive, by weight;

a flavoring agent, wherein the concentration of the flavoring agent in the composition is 0-2.5%, inclusive, by weight; and

a sweetener, wherein the concentration of the sweetener in the composition is 0-0.03%, inclusive, by weight.

In certain embodiments, the composition comprises:

(i) a polyvinylpyrrolidone copolymer (e.g., PVP-VA-64), wherein the concentration of the polyvinylpyrrolidone copolymer in the composition is 7-10%, inclusive, by weight;

(ii) a polyethylene glycol (PEG) (e.g., PEG400), wherein the concentration of the PEG in the composition is 40-65%, inclusive, by weight;

(iii) ethanol, wherein the concentration of ethanol in the composition is 15-20%, inclusive, by weight; and

(iv) water, wherein the concentration of water in the composition is 10-40%, inclusive, by weight.

In certain embodiments, the composition described in this paragraph further comprises:

a pH adjustment agent (e.g., citric acid), wherein the concentration of the pH adjustment agent in the composition is 0-0.12%, inclusive, by weight;

a cellulose derivative (e.g., HPC), wherein the concentration of the cellulose derivative in the composition is 1-1.5%, inclusive, by weight;

a polyol (e.g., glycerol), wherein the concentration of the polyol in the composition is 1.5-3%, inclusive, by weight;

a flavoring agent, wherein the concentration of the flavoring agent in the composition is 0-2.5%, inclusive, by weight; and

a sweetener, wherein the concentration of the sweetener in the composition is 0.01-0.03%, inclusive, by weight.

In certain embodiments, the composition comprises:

(i) a polyvinylpyrrolidone copolymer (e.g., PVP-VA-64), wherein the concentration of the polyvinylpyrrolidone copolymer in the composition is about 10%, by weight;

(ii) a polyethylene glycol (PEG) (e.g., PEG400), wherein the concentration of the PEG in the composition is about 61.8%, inclusive, by weight;

(iii) ethanol, wherein the concentration of ethanol in the composition is about 14%, inclusive, by weight; and

(iv) water, wherein the concentration of water in the composition is about 10%, inclusive, by weight.

In certain embodiments, the composition described in this paragraph further comprises:

a pH adjustment agent (e.g., citric acid), wherein the concentration of the pH adjustment agent in the composition is about 0.12%, by weight;

a polyol (e.g., glycerol), wherein the concentration of the polyol in the composition is about 1.5%, by weight;

a flavoring agent, wherein the concentration of the flavoring agent in the composition is about 2.5%, by weight; and

a sweetener, wherein the concentration of the sweetener in the composition is about 0.03%, by weight.

In yet another aspect of the invention, the disclosure provides compositions comprising: (i) one or both of a hydroxypropyl cellulose M and a hydroxypropyl cellulose G, wherein: when the composition comprises a hydroxypropyl cellulose M, the concentration of the hydroxypropyl cellulose M in the composition is between 0.1% and 5%, inclusive, by weight; when the composition comprises a hydroxypropyl cellulose G, the concentration of the hydroxypropyl cellulose Gin the composition is between 0.1% and 7%, inclusive, by weight; and when the composition comprises only one or both of a hydroxypropyl cellulose M and a hydroxypropyl cellulose G, the composition further comprises a polyvinylpyrrolidone copolymer, wherein the concentration of the polyvinylpyrrolidone copolymer in the composition is between 0% and 10%, inclusive, by weight; (ii) ethanol, a propanol, a butanol, or a mixture thereof, wherein the concentration of component (ii) thereof in the composition is between 10% and 80%, inclusive, by weight; and (iii) water, wherein the concentration of water in the composition is between 8% and 75%, inclusive, by weight; provided that the total concentration of all components of the composition is 100%, inclusive, by weight. Because the concentration of polyvinylpyrrolidone copolymer in the composition may be 0, the inclusion of polyvinylpyrrolidone copolymer in the composition is optional. When a composition described herein includes a component, wherein the concentration of the component in the composition is 0-YY %, wherein YY is a number between 0 and 100, exclusive, then the composition is either free of the component or includes the component at the concentration of not more than YY %. When the composition comprises only one or both of a hydroxypropyl cellulose M and a hydroxypropyl cellulose G, the composition optionally further comprises a polyvinylpyrrolidone copolymer, wherein the concentration of the polyvinylpyrrolidone copolymer in the composition is between 0% and 10%, inclusive, by weight. The composition may further comprise a cellulose derivative, pH adjustment agent, a PEG (polyethylene glycol), a polyol, and/or a polyvinylpyrrolidone homopolymer. In some embodiments, the composition further comprises a pH adjuster, a sweetener, a colorant, and/or a flavoring agent.

In certain embodiments, the composition comprises a hydroxypropyl cellulose. In some embodiments, the composition comprises both of a hydroxypropyl cellulose M and a hydroxypropyl cellulose G. In some embodiments, the composition comprises a hydroxypropyl cellulose M and a polyvinylpyrrolidone copolymer. In some embodiments, the composition comprises a hydroxypropyl cellulose G and a polyvinylpyrrolidone copolymer. In certain embodiments, the composition comprises a hydroxypropyl cellulose M, hydroxypropyl cellulose G, and a polyvinylpyrrolidone copolymer. In some embodiments, the composition comprises a hydroxypropyl cellulose M. In some embodiments, the composition comprises a hydroxypropyl cellulose G.

In some embodiments, the concentration of the hydroxypropyl cellulose M in the composition is between 0.1% and 5%, inclusive, by weight. In some embodiments, the concentration of the hydroxypropyl cellulose M in the composition is between 0.3% and 4%, inclusive, by weight. In some embodiments, the concentration of the hydroxypropyl cellulose M in the composition is between 0.3% and 2.7%, inclusive, by weight. In some embodiments, the concentration of the hydroxypropyl cellulose M in the composition is between 0.3% and 1.8%, inclusive, by weight.

In some embodiments, the concentration of the hydroxypropyl cellulose Gin the composition is between 0.1% and 7%, inclusive, by weight. In some embodiments, the concentration of the hydroxypropyl cellulose G in the composition is between 0.19% and 6.5%, inclusive, by weight. In some embodiments, the concentration of the hydroxypropyl cellulose G in the composition is between 0.6% and 3.5%, inclusive, by weight. In some embodiments, the concentration of the hydroxypropyl cellulose Gin the composition is between 0.6% and 1.8%, inclusive, by weight.

In some embodiments, the composition further comprises a polyvinylpyrrolidone copolymer. In some embodiments, the polyvinylpyrrolidone copolymer is poly(vinylpyrrolidone-co-vinyl acetate) 64. In certain embodiments, the polyvinylpyrrolidone copolymer is poly(vinylpyrrolidone-co-vinyl acetate) 64. In certain embodiments, the polyvinylpyrrolidone copolymer is select from the group of PVP/VA E-335, PVP/VA E-535, PVP/VA E-635, PVP/VA E-735, PVP/VA 1-335, PVP/VA 1-735, PVP/VA S-630, PVP/VA S-630L, PVP/VA W-535, PVP/VA W-635, PVP/VA W-735, Luvitec® VA 64 W, Luvitec® VA 64 P, Luvitec® VPC 55 K 65 W, Kollidon® VA 64, and Collacral® Val.

In certain embodiments, a provided composition is substantially free of polyvinylpyrrolidone copolymers. In certain embodiments, the concentration of the polyvinylpyrrolidone copolymer in the composition is 0% by weight. In certain embodiments, the concentration of the polyvinylpyrrolidone copolymer in the composition is between 0% and 10%, inclusive, by weight. In some embodiments, the concentration of the polyvinylpyrrolidone copolymer in the composition is between 0.02% and 4%, inclusive, by weight. In some embodiments, the concentration of the polyvinylpyrrolidone copolymer in the composition is between 0% and 3.5%, inclusive, by weight. In certain embodiments, the concentration of the polyvinylpyrrolidone copolymer in the composition is between 0% and 2%, inclusive, by weight. In certain embodiments, the concentration of the polyvinylpyrrolidone copolymer in the composition is between 0% and 1.8%, inclusive, by weight. In certain embodiments, the concentration of the polyvinylpyrrolidone copolymer in the composition is between 0.5% and 1.8%, inclusive, by weight.

In certain embodiments, component (ii) of the composition is ethanol. In certain embodiments, component (ii) of the composition is a propanol (e.g., 1-propanol, 2-propanol). In certain embodiments, component (ii) of the composition is a butanol (e.g., 1-butanol, 2-butanol, isobutanol, tert-butanol). In certain embodiments, component (ii) is a mixture of ethanol and a propanol. In certain embodiments, component (ii) is a mixture of ethanol and a butanol. In certain embodiments, component (ii) is a mixture of a propanol and a butanol. In certain embodiments, component (ii) is a 1:1 mixture of ethanol and a propanol. In certain embodiments, component (ii) is a 1:1 mixture of ethanol and a butanol. In certain embodiments, component (ii) is a 1:1 mixture of a propanol and a butanol.

In some embodiments, the concentration of component (ii) in the composition is between 10% and 80%, inclusive, by weight. In some embodiments, the concentration of component (ii) in the composition is between 15% and 77%, inclusive, by weight. In some embodiments, the concentration of component (ii) in the composition is between 15% and 40%, inclusive, by weight. In certain embodiments, the concentration of component (ii) in the composition is between 26% and 40%, inclusive, by weight. In certain embodiments, the concentration of component (ii) in the composition is between 33% and 40%, inclusive, by weight. In certain embodiments, the concentration of component (ii) in the composition is between 36% and 40%, inclusive, by weight.

In some embodiments, the concentration of water in the composition is between 8% and 75%, inclusive, by weight. In some embodiments, the concentration of water in the composition is between 8% and 60%, inclusive, by weight. In some embodiments, the concentration of water in the composition is between 8.9% and 59.5%, inclusive, by weight. In some embodiments, the concentration of water in the composition is between 50% and 66%, inclusive, by weight. In some embodiments, the concentration of water in the composition is between 50% and 58%, inclusive, by weight. In some embodiments, the concentration of water in the composition is between 51% and 58%, inclusive, by weight.

In certain embodiments, a provided composition further comprises a polyvinylpyrrolidone homopolymer. In certain embodiments, the polyvinylpyrrolidone homopolymer is polyvinylpyrrolidone K12, polyvinylpyrrolidone K15, polyvinylpyrrolidone K25, polyvinylpyrrolidone K29, polyvinylpyrrolidone K30, polyvinylpyrrolidone K60, polyvinylpyrrolidone K90, or polyvinylpyrrolidone K120. In certain embodiments, the polyvinylpyrrolidone homopolymer is polyvinylpyrrolidone K29. In certain embodiments, the polyvinylpyrrolidone homopolymer is polyvinylpyrrolidone K30.

In some embodiments, the concentration of the polyvinylpyrrolidone homopolymer in the composition is between 0 to 10%, inclusive, by weight. In some embodiments, the concentration of the polyvinylpyrrolidone homopolymer in the composition is between 0.1 to 10%, inclusive, by weight. In some embodiments, the concentration of the polyvinylpyrrolidone homopolymer in the composition is between 0.1% and 3.5%, inclusive, by weight. In some embodiments, the concentration of the polyvinylpyrrolidone homopolymer in the composition is between 0% and 1.8%, inclusive, by weight. In some embodiments, the concentration of the polyvinylpyrrolidone homopolymer in the composition is between 0.1% and 1.8%, inclusive, by weight.

In certain embodiments, the composition further comprises a cellulose derivative. In some embodiments, the cellulose derivative is selected from the group consisting of methylcellulose, carboxymethyl cellulose, ethyl cellulose, ethyl methyl cellulose, hydroxypropyl cellulose, and hydroxypropyl methylcellulose. In certain embodiments, the cellulose derivative is methylcellulose. In certain embodiments, the cellulose derivative is hydroxypropyl cellulose. In some embodiments, the cellulose derivative is an additional hydroxypropyl cellulose, wherein the additional hydroxypropyl cellulose is not hydroxypropyl cellulose M (HPC-M) or hydroxypropyl cellulose G (HPC-G) and is optionally hydroxypropyl cellulose E (HPC-E), hydroxypropyl cellulose H (HPC-H), or hydroxypropyl cellulose J (HPC-J). In some embodiments, the cellulose derivative is hydroxypropyl cellulose E. In some embodiments, the cellulose derivative is hydroxypropyl cellulose H. In some embodiments, the cellulose derivative is hydroxypropyl cellulose J.

In certain embodiments, the concentration of the cellulose derivative in the composition is between 0% to 10%, inclusive, by weight. In certain embodiments, the concentration of the cellulose derivative in the composition is between 0.01% to 10%, inclusive, by weight. In certain embodiments, the concentration of the cellulose derivative in the composition is between 0.03% to 3%, inclusive, by weight. In some embodiments, the concentration of the cellulose derivative in the composition is between 0.03% and 1.84%, inclusive, by weight. In certain embodiments, the concentration of the cellulose derivative in the composition is between 0% and 1.8%, inclusive, by weight. In certain embodiments, the concentration of the cellulose derivative in the composition is between 0.3% and 1.8%, inclusive, by weight.

In certain embodiments, the composition is substantially free of cellulose derivatives.

In some embodiments, the composition further comprises a pH adjustment agent. In certain embodiments, the pH adjustment agent is an inorganic acid. In some embodiments, the pH adjustment agent is an inorganic base. In certain embodiments, the pH adjustment agent is an organic acid. In some embodiments, the pH adjustment agent is an organic base. In some embodiments, the pH adjustment agent is citric acid. In some embodiments, the pH adjustment agent is salicylic acid. In certain embodiments, the concentration of the pH adjustment agent in the composition is between 0% and 2.5%, inclusive, by weight. In certain embodiments, the concentration of the pH adjustment agent in the composition is between 0.017% and 2.5%, inclusive, by weight. In certain embodiments, the concentration of the pH adjustment agent in the composition is between 0% and 1.7%, inclusive, by weight. In certain embodiments, the concentration of the pH adjustment agent in the composition is between 0.1% and 1.7%, inclusive, by weight.

In some embodiments, the composition further comprises a PEG. In some embodiments, the PEG in the composition is a PEG between PEG200 and PEG600, inclusive. In certain embodiments, the PEG is selected from the group consisting of PEG200, PEG250, PEG300, PEG350, PEG400, PEG450, PEG500, PEG550, and PEG600. In some embodiments, the PEG is PEG400.

In some embodiments, the concentration of the PEG in the composition is between 0.1% and 18%, inclusive, by weight. In some embodiments, the concentration of the PEG in the composition is between 0.6% and 8.5%, inclusive, by weight. In some embodiments, the concentration of the PEG in the composition is between 0.6% and 7.9%, inclusive, by weight. In some embodiments, the concentration of the PEG in the composition is between 0.6% and 1.8%, inclusive, by weight.

In certain embodiments, the composition further comprises a polyol. In some embodiments, the polyol is selected from the group of glycerol, erythritol, isomalt, maltitol, sorbitol, lactitol, mannitol, xylitol, trimethylolpropane, and pentaerythritol. In certain embodiments, the polyol is glycerol.

In some embodiments, the composition further comprises a polyol wherein the concentration of the polyol is between 0% to 3.8%, inclusive, by weight. In some embodiments, the composition further comprises a polyol wherein the concentration of the polyol is between 0.038% to 3.8%, inclusive, by weight. In some embodiments, the concentration of the polyol in the composition is between 0% and 3.5%, inclusive, by weight. In some embodiments, the concentration of the polyol in the composition is between 0.038% and 3.5%, inclusive, by weight. In some embodiments, the concentration of the polyol in the composition is between 1.5% and 3.5%, inclusive, by weight. In some embodiments, the concentration of the polyol in the composition is between 1.5% and 3%, inclusive, by weight.

In certain embodiments, a provided composition further comprises a sweetener. In some embodiments, the concentration of the sweetener in the composition is between 0.01% and 5%, inclusive, by weight. In some embodiments, the concentration of the sweetener in the composition is between 0.01% and 1%, inclusive, by weight. In some embodiments, the concentration of the sweetener in the composition is between 0.01% and 0.03%, inclusive, by weight.

In certain embodiments, a provided composition further comprises a pH adjuster. In certain embodiments, the pH adjuster is an inorganic acid. In some embodiments, the pH adjuster is an inorganic base (e.g., alkali metal hydroxide). In certain embodiments, the pH adjuster is an organic acid. In some embodiments, the pH adjuster is an organic base. In some embodiments, the pH adjuster is NaOH. In certain embodiments, the pH adjuster is HCl. In some embodiments, the concentration of the pH adjuster in the composition is between 0.007% and 5%, inclusive, by weight. In some embodiments, the concentration of the pH adjuster in the composition is between 0.007% and 2.5%, inclusive, by weight. In some embodiments, the concentration of the pH adjuster in the composition is between 0.007% and 1.7%, inclusive, by weight. In some embodiments, the concentration of the pH adjuster in the composition is between 0.007% and 0.12%, inclusive, by weight.

In certain embodiments, a composition further comprises a colorant. In some embodiments, the concentration of the colorant in the composition is between 0.001% and 5%, inclusive, by weight. In some embodiments, the concentration of the colorant in the composition is between 0.001% and 2.5%, inclusive, by weight. In some embodiments, the concentration of the colorant in the composition is between 0.1% and 1%, inclusive, by weight.

In certain embodiments, a provided composition further comprises a flavoring agent. In some embodiments, the concentration of the flavoring agent in the composition is between 0.001% and 5%, inclusive, by weight. In some embodiments, the concentration of the flavoring agent in the composition is between 0.001% and 2.5%, inclusive, by weight. In some embodiments, the concentration of the flavoring agent in the composition is between 0.1% and 1%, inclusive, by weight.

In certain embodiments, the composition further comprises a cyclodextrin. In some embodiments, the cyclodextrin is selected from the group consisting of alpha-cyclodextrin, beta-cyclodextrin, gamma-cyclodextrin, methyl-beta-cyclodextrin, hydroxypropyl-beta-cyclodextrin, and hydroxypropyl-gamma-cyclodextrin. In some embodiments, the cyclodextrin is selected from the group consisting of Cavasol® W7 HP, Cavasol® W6 HP, Cavasol® W8 HP, Cavasol® W7 M, Cavitron® W7 HP5, Cavitron® W7 HP7, Cavamax® W8 Cavamax® W6, and Cavamax® W7.

In some embodiments, the concentration of cyclodextrin in the composition is between 0 to 30%, inclusive, by weight. In some embodiments, the concentration of cyclodextrin in the composition is between 0.1 to 30%, inclusive, by weight. In some embodiments, the concentration of the cyclodextrin in the composition is between 0.3% and 29%, inclusive, by weight. In some embodiments, the concentration of the cyclodextrin in the composition is between 3% and 9%, inclusive, by weight. In some embodiments, the concentration of cyclodextrin in the composition is between 0 to 5.5%, inclusive, by weight. In some embodiments, the concentration of the cyclodextrin in the composition is between 25% and 30%, inclusive, by weight. In certain embodiments, the cyclodextrin increases the solubility of the active agent in the composition.

In some embodiments, the composition contains a polyacrylic acid. In certain embodiments, the polyacrylic acid in the composition is a Carbomer® polymer. In certain embodiments, the Carbomer® polymer is selected from the group consisting of 910, 934, 940, 941, 934P, and 962. In some embodiments, the polyacrylic acid is a Carbopol® polymer. In certain embodiments, the Carbopol® polymer is selected from the group consisting of 71G NF, 971P NF, 974P NF, 980 NF, 981 NF, 5984 EP, ETD 2020 NF, Ultrez 10 NF, 934 NF, 934P NF, 940 NF, 941 NF, 1342 NF. In certain embodiments, the Carbopol® polymer is selected from the group consisting of 71G NF, 971P NF, 974P NF, and 934P NF. In certain embodiments, the Carbopol® polymer is selected from the group consisting of 971P NF, 974P NF, ETD 2020 NF, 980 NF, and 956. In certain embodiments, the polyacrylic acid is Carbopol® 971P NF polymer.

In certain embodiments, the concentration of the polyacrylic acid in the composition is between 0.1% and 10%, inclusive, by weight. In some embodiments, the concentration of the polyacrylic acid in the composition is between 0.1% and 9%, inclusive, by weight. In certain embodiments, the concentration of the polyacrylic acid in the composition is between 0.2% and 3%, inclusive, by weight. In some embodiments, the concentration of the polyacrylic acid in the composition is between 0.2% and 3%, inclusive, by weight. In some embodiments, the concentration of the polyacrylic acid in the composition is between 0.1% and 1%, inclusive, by weight. In certain embodiments, the concentration of the polyacrylic acid in the composition is between 0.2% and 0.9%, inclusive, by weight. in certain embodiments, the concentration of the polyacrylic acid in the composition is between 0% and 0.9%, inclusive, by weight.

In certain embodiments, the pH value of the composition under ambient conditions is between 2 and 7, inclusive. In certain embodiments, the pH value of the composition under ambient conditions is between 3 and 6, inclusive. In certain embodiments, the pH value of the composition under ambient conditions is between 4 and 5, inclusive.

In certain embodiments, the composition is as described in Tables 3, 4, and 7.

In certain embodiments, the composition comprises:

(i) hydroxypropyl cellulose M, wherein the concentration of the hydroxypropyl cellulose M in the composition is 0.3-4%, inclusive, by weight; hydroxypropyl cellulose G, wherein the concentration of the hydroxypropyl cellulose Gin the composition is 0.19-6.5%, inclusive, by weight; provided that the combined concentration of hydroxypropyl cellulose M and hydroxypropyl cellulose G in the composition is 0.4-8%, inclusive, by weight; a polyvinylpyrrolidone copolymer (e.g., PVP-VA-64), wherein the concentration of the polyvinylpyrrolidone copolymer in the composition is 0-2%, inclusive, by weight;

(ii) ethanol, wherein the concentration of ethanol in the composition is 15-77%, inclusive, by weight; and

(iii) water, wherein the concentration of water in the composition is 8.9-59.5%, inclusive, by weight.

In certain embodiments, the composition described in this paragraph further comprises:

a polyvinylpyrrolidone homopolymer (e.g., PVP-30), wherein the concentration of the polyvinylpyrrolidone homopolymer in the composition is 0-3.5%, inclusive, by weight;

a polyethylene glycol (PEG) (e.g., PEG400), wherein the concentration of the PEG in the composition is 0.6-7.9%, inclusive, by weight;

an additional hydroxypropyl cellulose, wherein the concentration of the additional hydroxypropyl cellulose in the composition is 0-1.84%, inclusive, by weight;

a cellulose derivative (e.g., MC), wherein the concentration of the cellulose derivative in the composition is 0-1.8%, inclusive, by weight;

a first pH adjustment agent (e.g., salicylic acid), wherein the concentration of the first pH adjustment agent in the composition is about 0-2.5%, inclusive, by weight; and a second pH adjustment agent (e.g., citric acid), wherein the concentration of the second pH adjustment agent in the composition is about 0-1.7%, inclusive, by weight;

a polyol (e.g., glycerol), wherein the concentration of the polyol in the composition is 0-3.5%, inclusive, by weight;

a cyclodextrin, wherein the concentration of the cyclodextrin in the composition is 0-5.5%, inclusive, by weight; and

a polyacrylic acid (e.g., Carbopol), wherein the concentration of the polyacrylic acid in the composition is 0-0.9%, inclusive, by weight.

In certain embodiments, the composition comprises:

(i) hydroxypropyl cellulose M, wherein the concentration of the hydroxypropyl cellulose M in the composition is 0.3-2.7%, inclusive, by weight; hydroxypropyl cellulose G, wherein the concentration of the hydroxypropyl cellulose Gin the composition is 0.6-3.5%, inclusive, by weight; provided that the combined concentration of hydroxypropyl cellulose M and hydroxypropyl cellulose G in the composition is 0.4-4.6%, inclusive, by weight; a polyvinylpyrrolidone copolymer (e.g., PVP-VA-64), wherein the concentration of the polyvinylpyrrolidone copolymer in the composition is 0-2%, inclusive, by weight;

(ii) ethanol, wherein the concentration of ethanol in the composition is 33-40%, inclusive, by weight; and

(iii) water, wherein the concentration of water in the composition is 50-58%, inclusive, by weight.

In certain embodiments, the composition described in this paragraph further comprises:

a polyvinylpyrrolidone homopolymer (e.g., PVP-30), wherein the concentration of the polyvinylpyrrolidone homopolymer in the composition is 0-1.8%, inclusive, by weight;

a polyethylene glycol (PEG) (e.g., PEG400), wherein the concentration of the PEG in the composition is 0-1.8%, inclusive, by weight;

an additional hydroxypropyl cellulose, wherein the concentration of the additional hydroxypropyl cellulose in the composition is 0-1.84%, inclusive, by weight;

a cellulose derivative (e.g., MC), wherein the concentration of the cellulose derivative in the composition is 0-1.8%, inclusive, by weight;

a first pH adjustment agent (e.g., salicylic acid), wherein the concentration of the first pH adjustment agent in the composition is about 0-2.5%, inclusive, by weight; and a second pH adjustment agent (e.g., citric acid), wherein the concentration of the second pH adjustment agent in the composition is about 0.1-1.7%, inclusive, by weight;

a polyol (e.g., glycerol), wherein the concentration of the polyol in the composition is 0-3.5%, inclusive, by weight;

a cyclodextrin, wherein the concentration of the cyclodextrin in the composition is 0-5.5%, inclusive, by weight; and

a polyacrylic acid (e.g., Carbopol), wherein the concentration of the polyacrylic acid in the composition is 0-0.9%, inclusive, by weight.

In certain embodiments, the composition comprises:

(i) hydroxypropyl cellulose M, wherein the concentration of the hydroxypropyl cellulose M in the composition is 0.3-1.8%, inclusive, by weight; hydroxypropyl cellulose G, wherein the concentration of the hydroxypropyl cellulose Gin the composition is 0.6-1.8%, inclusive, by weight; provided that the combined concentration of hydroxypropyl cellulose M and hydroxypropyl cellulose G in the composition is 0.4-3.6%, inclusive, by weight; a polyvinylpyrrolidone copolymer (e.g., PVP-VA-64), wherein the concentration of the polyvinylpyrrolidone copolymer in the composition is 0-2%, inclusive, by weight;

(ii) ethanol, wherein the concentration of ethanol in the composition is 36-40%, inclusive, by weight; and

(iii) water, wherein the concentration of water in the composition is 51-58%, inclusive, by weight.

In certain embodiments, the composition described in this paragraph further comprises:

a polyethylene glycol (PEG) (e.g., PEG400), wherein the concentration of the PEG in the composition is 0.6-1.8%, inclusive, by weight;

an additional hydroxypropyl cellulose, wherein the concentration of the additional hydroxypropyl cellulose in the composition is 0-1.84%, inclusive, by weight;

a cellulose derivative (e.g., MC), wherein the concentration of the cellulose derivative in the composition is 0-1.8%, inclusive, by weight;

a pH adjustment agent (e.g., citric acid), wherein the concentration of the pH adjustment agent in the composition is about 0.1-1.7%, inclusive, by weight; and

a polyol (e.g., glycerol), wherein the concentration of the polyol in the composition is 1.5-3.5%, inclusive, by weight.

In certain embodiments, the composition comprises:

(i) hydroxypropyl cellulose M, wherein the concentration of the hydroxypropyl cellulose M in the composition is about 0.4%, by weight; hydroxypropyl cellulose G, wherein the concentration of the hydroxypropyl cellulose G in the composition is about 0.9%, by weight;

(ii) ethanol, wherein the concentration of ethanol in the composition is about 39%, by weight; and

(iii) water, wherein the concentration of water in the composition is about 57.9%, by weight.

In certain embodiments, the composition described in this paragraph further comprises a polyethylene glycol (PEG) (e.g., PEG400), wherein the concentration of the PEG in the composition is about 1.8%, by weight.

In certain embodiments, the composition comprises:

(i) hydroxypropyl cellulose M, wherein the concentration of the hydroxypropyl cellulose M in the composition is about 0.6%, by weight; hydroxypropyl cellulose G, wherein the concentration of the hydroxypropyl cellulose G in the composition is about 0.9%, by weight;

(ii) ethanol, wherein the concentration of ethanol in the composition is about 39%, by weight; and

(iii) water, wherein the concentration of water in the composition is about 56%, by weight.

In certain embodiments, the composition described in this paragraph further comprises:

a polyethylene glycol (PEG) (e.g., PEG400), wherein the concentration of the PEG in the composition is about 1.8%, by weight;

a pH adjustment agent (e.g., citric acid), wherein the concentration of the pH adjustment agent in the composition is about 1.7%, by weight.

The embodiments of the invention disclosed a composition that is presented as a liquid or gel composition. The embodiments of the invention disclosed as a liquid composition may be presented as a solution, an emulsion, a dispersion or a suspension. The embodiments of the invention disclosed as a gel composition may be presented as homogeneous or heterogeneous composition.

The embodiments of the invention presented in the form of a liquid or gel is applied at, on and/or into the mucosal surface by means of a suitable applicator such as a spray, a dropper, a brush, a roll-on, a spatula, a pallet or a pen. Once applied at, on and/or into the mucosal surface, the composition directly contacts and covers an area of the mucosal surface, potentiating the local release of the active agents for topical and/or systemic delivery.

In the present invention, each active agent may be incorporated in the composition and may be in solution or in the form of a suspension. Depending upon the qualitative and quantitative composition of the formulation chosen, the active agent may be released from the composition over a period of time (i.e. sustained release) or immediately. The present invention can be used in the treatment of both humans and animals.

In certain embodiments, the evaporation or partial evaporation of organic and/or inorganic solvents could start occurring immediately following the delivery of the composition and/or once the composition is in contact with mucosal milieu.

In the context of this invention, solvent(s) refers to compound(s) that has the ability to dissolve, suspend or extract other materials usually without causing a chemical change to the other material or solvent.

In the context of this invention, organic solvent(s) refers to compound(s) that contain carbon bonds and in which at least one carbon atom is covalently linked to an atom of hydrogen molecule.

In the context of this invention, inorganic solvent(s) refers to compound(s) that do not contain both carbon and hydrogen molecules.

In certain embodiments, the evaporation or partial evaporation of organic and/or inorganic solvents could lead to increase of local excipient and/or active agent(s) concentration, and hence increase of polymer concentration. In certain embodiments, the evaporation or partial evaporation of organic and/or inorganic solvents could increase of polymer concentration and optionally promote changes in the local viscosity of the composition.

In the embodiments of the invention, the non-volatile component of the composition prevents the active agent(s) from precipitating when the volatile solvent component evaporates or partially evaporates.

In the context of this invention, non-volatile component refers to a substance that does not readily evaporate into a gas under existing conditions, i.e., a material that exerts a low vapor pressure and has a slow rate of evaporation.

The embodiments of the invention disclosed herein are generally prepared in an aqueous solvent phase or hydroalcoholic solvent phase.

In certain embodiments, the organic and inorganic solvents could contribute for the solubilization of active agents and excipients in the composition, in particular, but not limited to water-soluble active agents, water-soluble excipients, non-water soluble active agents, non-water soluble excipients, amphiphilic active agents and/or amphiphilic excipients.

In certain embodiments, the organic and inorganic solvents could also contribute to the formation of the polymeric film (polymer matrices) once the composition is applied at and/or on the mucosal membrane following solvent evaporation, absorption into and/or dissipation on the substrate.

In certain embodiments, the organic solvent is one or a mixture of alcohols, such as, but not limited to alcohols, glycol ethers, methyl acetate, ethyl acetate, ketones, esters, and/or glycol ether/esters. In certain embodiments, the organic solvent is preferably one or a mixture of ethanol, propanol and/or butanol. In certain embodiments, the organic solvent is preferably ethanol and represents about 0% to about 80% by weight of the composition. In certain embodiments, the ethanol preferably represents about 4% to about 65% by weight of the composition. In certain embodiments, the ethanol preferably represents about 4% to about 40% by weight of the composition.

In certain embodiments, the organic solvent is ethanol and the inorganic solvent is water. In certain embodiments, the water represents about 5% to about 75% by weight of the composition. In certain embodiments, the water represents about 10% to about 65% by weight of the composition.

In certain embodiments, a provided mucoadhesive composition further comprises an organic and inorganic solvent mixture at different weight ratios. In certain embodiments, the organic and inorganic parts are ethanol and water and are presented at weight ratios of about 0 to about 9. In certain embodiments, the ethanol and water are presented at weight ratios of about 0.3 to about 1.7.

In the embodiments of this invention, the optimal combination of organic and/or inorganic solvents with functional excipients, in particular, but not limited to film-forming, mucoadhesive and plasticizers agents can favor the modulation of viscosity, the film-forming capability and mucoadhesiveness for a provided composition.

“Film forming (agents)” as used herein refers to substances, for example polymers, capable of forming polymer matrices comprised with polymer chains that can temporarily entrap active agents before their release from the composition. The “Film forming (agents)” could be classified as nonreactive or reactive film-forming agent. Nonreactive film-forming agent is for example the agent capable to promote a film-forming as a result of evaporation of the solvent; Reactive film-forming agent rely, for example, on reactive agents capable of promoting film formation as a result of chemical transformations.

Nonreactive film-forming agents often include polymers with film-forming capacity. The film-forming polymers include, but not limit to, acrylic polymers or copolymers, cellulose derivatives, and other polymers. Preferred film-forming polymers include a non-ionic copolymer of methacrylate derivatives, copolymers of monoalkyl esters of poly (methyl vinyl ether/maleic acid), poly vinyl acetate, cellulose acetate, polyvinyl alcohol, povidone, povidone vinyl acetate, copovidone, hydroxypropyl methylcellulose, hydroxyethyl cellulose, methylcellulose, hydroxypropyl cellulose.

Mucoadhesive agent(s) as used herein is referred to a natural or a synthetic agent(s) that can adhere to a mucosa or mucosal-like surfaces. In the context of this invention, mucoadhesion is assessed using Texture Analyser, such as TA.XT.plus from Stable Micro System (UK), equipped with a mucoadhesion support (such as mucoadhesion rig A/MUC), a probe (such as P10—10.0 cm diameter) and a load cell (for example 5 kg or 30 kg load cell). Briefly, the probe is fixed in the equipment and vertically compress a substrate (pre-hydrated natural or synthetic gut or pre-hydrated natural or synthetic gut with composition to be tested). After compressing in a controlled manner (pre-defined constant force and during a specific period-of-time), the probe returns to the initial position. The protocol of mucoadhesion test records the adhesiveness force, the work of adhesion and the duration of detachment between the composition and pre-hydrated natural or synthetic gut. The obtained value of mucoadhesion is an outcome of the difference between the adhesiveness obtained for the composition with pre-hydrated gut and the value obtained only with pre-hydrated gut (without composition). An example of the settings established for the mucoadhesion method using texture analyzer are as follows: Pre-test Speed (0.80 mm/Sec); Test Speed (0.10 mm/Sec); Post Test Speed (0.80 mm/Sec); Applied Force (2.50 g); Return Distance (10.0 mm); Contact Time (60.0 Sec); Auto Trigger Type; Trigger Force (5.0 g).

Mucoadhesive component, agent, or excipient can be polymers.

Mucoadhesive polymers are generally water-soluble or water insoluble polymers, which are swellable networks. For example, mucoadhesive polymers could hold optimal polarity to allow sufficient wetting by the mucus and optimal fluidity that permits the mutual adsorption and interpenetration of polymer and mucus.

Mucoadhesive polymers may be of natural or synthetic origin.

Natural polymers may be protein-based or Polysaccharides polymers. Examples of natural-based polymers include, but not limits, collagen, albumin, gelatin. Polysacharides, include, but are not limited to, Alginates, Cyclodextrins, Chitosan, Dextran, Agarose, Hyaluronic acid, Starch, Cellulose.

Synthetic polymers may be distinguished in biodegradable and non-biodegradable polymers.

Biodegradable polymers are generally categorized as Polyesters, including (but not limited to) Polylactic acid, Polyglycolic acid, Polyhydroxyl butyrate, Polycaprolactone, Poly Doxanones; or Polyanhydride, including (but not limited to) Polyadipic acid, Polyterphthalic acid, Polysebacic acid and Various copolymers; or Polyamides including (but not limited to) Poly iminocarbonates, Poly amino acids; or Phosphorous Based polymers including (but not limited to) Polyphosphonates, Polyphosphazenes; or others such as Poly cyanocrylates, Poly urethanes, Poly ortho esters, Polyacetals.

Non-Biodegradable polymers are generally categorized as Cellulose derivatives including (but not limited to) Carboxymethylcellulose (CMC), Ethyl cellulose (EC), hydroxyethylcellulose (HEC), Cellulose acetate, hydroxypropyl methylcellulose (HPMC), methylcellulose (MC), hydroxylpropyl cellulose (HPC), ethyl hydroxyethyl cellulose (EHEC), methyl hydroxyethyl cellulose (MHEC); or Silicones including (but not limited to) Polydimethyl siloxanes, Colloidal silica, Polymethacrylates; or Others such as copolymers of monoalkyl esters of poly (methyl vinyl ether/maleic acid) (PVM/MA), polyvinyl alcohol (PVA), povidone (PVP (e.g., polyvinylpyrrolidone K29, polyvinylpyrrolidone K30), povidone vinyl acetate (copovidone (e.g., PVP-VA-64, also referred to as poly(vinylpyrrolidone-co-vinyl acetate) 64)), ethylene-vinyl acetate (EVA), polyethylene glycols (PEG), Poloxamines and Poloxamers.

In certain embodiments, a provided composition is prepared also with one or a combination of different components such as plasticizer, pH adjustment additive, taste-masking additive, sweetener, flavor enhancement additive, cooling (after-taste) additive, colorant, surfactant, preservative, antioxidant and/or penetration enhancement additive.

In certain embodiments, a provided composition further comprises a plasticizer. Exemplary plasticizers include, but are not limited to, phthalate derivatives (e.g., dimethyl phthalate, diethyl phthalate, dibutyl phthalate), citrate derivatives (e.g., tributylcitrate, triethylcitrate, acetyl citrate, citric acid), polyalkylene oxides (e.g., polyethylene glycols, polypropylene glycols, polyethylene-propylene glycols), glycerol, glycerol monoacetate, glycerol diacetate, triacetate, triacetin, polysorbate, cetyl alcohol, propylene glycol, sorbitol, sodium diethylsulfosuccinate, and castor oil. In certain embodiments, the plasticizer is a Glycerol. In certain embodiments, Glycerol represents about 0% to about 20% based on the weight of all the components of the composition. In certain embodiments, glycerol represents about 0% to about 5% based on the weight of all the components of the film.

In certain embodiments, a provided composition also comprises a pH adjuster and/or pH adjustment agent. pH adjuster and/or pH adjustment agent can be added to modulate the pH. The pH can have an important role in the dissolution and stabilization of the components in the formulation, but also in their absorption through the mucosal surface. Exemplary pH adjusters and pH adjustment agents, include, but are not limited to acidifying agents, alkalizing agents, pH adjustment buffers, acid/acid salt system.

Acidifying agents are used to stabilize the product providing appropriate acidic pH. Examples of acidifying agents, include, but are not limited to adipic acid, citric acid, acetic acid, bonzoic acid, butyric acid, lactic acid, levulinic acid, oleic acid, sorbic acid, stearic acid, tartaric acid, tanic acid, malic acid, fumaric acid, hydrochloric acid, nitric acid and/or salicylic acid.

Alkalizing agents are used to stabilize the product providing appropriate alcaline pH. Examples of Alkalizing agents, include, but are not limited to sodium hydroxide, potassium hydroxide, sodium bicarbonate, ammonia solution, potassium citrate, sodium citrate, ammonia carbonate, triethanolamine, sodium borate and/or trolamine.

Examples of pH adjustment buffers, include, but are not limited to citrate buffers, phosphate buffers, acetate buffers, carbonate buffers, ammonia buffers, borate buffers, lactate buffers, ethanolamine buffers, glycine buffers, methionine buffers, glutamate buffers and succinate buffers.

Examples of acid/acid salt systems include, but are not limited to, citric acid/citric acid salts (e.g. sodium citrate, potassium citrate), citric acid/phosphoric acid salts (e.g. sodium aluminum phosphate, sodium monobasic phosphate, sodium dibasic phosphate, sodium tribasic phosphate, potassium tribasic phosphate, potassium monobasic phosphate, potassium dibasic phosphate), citric acid/tartaric acid salts (e.g. sodium tartrate, potassium tartrate), citric acid/boric acid salts (e.g. sodium borate, potassium borate), citric acid/malic acid salts (e.g. sodium malate, potassium malate), citric acid/maleic acid salts (e.g. sodium maleate, potassium maleate), tartaric acid/citric acid salts (e.g. sodium citrate, potassium citrate), tartaric acid/phosphoric acid salts (e.g. sodium aluminium phosphate, sodium monobasic phosphate, sodium dibasic phosphate, sodium tribasic phosphate, potassium tribasic phosphate, potassium monobasic phosphate, potassium dibasic phosphate), tartaric acid/tartaric acid salts (e.g. sodium tartrate, potassium tartrate), tartaric acid/boric acid salts (e.g. sodium borate, potassium borate), tartaric acid/malic acid salts (e.g. sodium malate, potassium malate), tartaric acid/maleic acid salts (e.g. sodium maleate, potassium maleate), boric acid/citric acid salts (e.g. sodium citrate, potassium citrate), boric acid/phosphoric acid salts (e.g. sodium aluminium phosphate, sodium monobasic phosphate, sodium dibasic phosphate, sodium tribasic phosphate, potassium tribasic phosphate, potassium monobasic phosphate, potassium dibasic phosphate), boric acid/tartaric acid salts (e.g. sodium tartrate, potassium tartrate), boric acid/boric acid salts (e.g. sodium borate, potassium borate), boric acid/malic acid salts (e.g. sodium malate, potassium malate), boric acid/maleic acid salts (e.g. sodium maleate, potassium maleate), malic acid/citric acid salts (e.g. sodium citrate, potassium citrate), malic acid/phosphoric acid salts (e.g. sodium aluminium phosphate, sodium monobasic phosphate, sodium dibasic phosphate, sodium tribasic phosphate, potassium tribasic phosphate, potassium monobasic phosphate, potassium dibasic phosphate), malic acid/tartaric acid salts (e.g. sodium tartrate, potassium tartrate), malic acid/boric acid salts (e.g. sodium borate, potassium borate), malic acid/malic acid salts (e.g. sodium malate, potassium malate), malic acid/maleic acid salts (e.g. sodium maleate, potassium maleate), maleic acid/citric acid salts (e.g. sodium citrate, potassium citrate), maleic acid/phosphoric acid salts (e.g. sodium aluminium phosphate, sodium monobasic phosphate, sodium dibasic phosphate, sodium tribasic phosphate, potassium tribasic phosphate, potassium monobasic phosphate, potassium dibasic phosphate), maleic acid/tartaric acid salts (e.g. sodium tartrate, potassium tartrate), maleic acid/boric acid salts (e.g. sodium borate, potassium borate), maleic acid/malic acid salts (e.g. sodium malate, potassium malate), maleic acid/maleic acid salts (e.g. sodium maleate, potassium maleate).

In certain embodiments, the pH adjuster and/or pH adjustment agent are one or combination of salicylic acid, citric acid, sodium hydroxide and/or hydrochloric acid. In certain embodiments, the pH adjuster and/or pH adjustment agent represents about 0% to about 5% by weight of the composition. In certain embodiments, a pH adjuster and/or pH adjustment agent represents 0.007% to about 1.7% by weight of the composition.

In certain embodiments, a provided composition further comprises citric acid. Citric acid can act as a plasticizer and also as a pH adjustment additive and also as a penetration enhancer and also as a flavoring agent. In certain embodiments, the composition further comprises citric acid as about 0% to about 2% based on the weight of all the components of the composition. In certain embodiments, the composition further comprises citric acid as about 0.01% to about 1.7% based on the weight of all the components of the composition. In certain embodiments, the composition further comprises citric acid preferably as about 0.1% to about 0.7% based on the weight of all the components of the composition.

In certain embodiments, a provided composition further comprises taste-masking. Taste-masking agents can be added to ameliorate the general organoleptic characteristics of the film-forming and mucoadhesive compositions. In certain embodiments, taste-masking agents may be used to mask unpleasant taste of some components. The main taste sensations include metallic, acidic, alkaline, salty, sweet, bitter and sour. Exemplary of taste-masking agents include, but are not limited to, menthol, peppermint oil, L-Mentol, cyclodextrins, glycerol, maltodextrins, ion-exchange resins, amino acids, gelatin, gelatinized starch, liposomes, lecithin or lecithin-like substances and salts. In certain embodiments, cyclodextrin is used as a taste-masking agent. The amount of taste-masking added can vary with the taste-masking employed. In certain embodiments, cyclodextrins are used as taste-masking agent and comprises about 0% to about 50% based on the dry weight of all the components of the composition. In certain embodiments, the taste-masking agent represents 0% to about 5% based on the dry weight of all the components of the composition.

In certain embodiments, a provided composition further comprises sweetener. Sweeteners can be used to improve palatability and are usually classified as natural or artificial. A sweetener may be a natural sweetener or artificial sweetener. Exemplary natural sweeteners include, but are not limited to, dextrose, fructose, glucose, liquid glucose, maltose, rebiana, glycyrrhizin, thaumatin, sorbitol, mannitol, isomalt, glycerol, maltitol, xylitol, and erythritol. Exemplary artificial sweeteners include, but are not limited to, saccharin, cyclamate, aspartame, acesulfame-K, sucralose, alitame and neotame. In certain embodiments, sucralose is used as a sweetener. In certain embodiments, one or combination of neohespiridin dihydrochalcone, glycerol and/or sucralose are used as sweeteners.

The amount of sweetener added can vary with the sweetener employed. In some embodiments, the concentration of the sweetener in the composition is between 0.01% and 5%, inclusive, by weight. In some embodiments, the concentration of the sweetener in the composition is between 0.01% and 1%, inclusive, by weight. In some embodiments, the concentration of the sweetener in the composition is between 0.5% and 1%, inclusive, by weight. In certain embodiments, the composition further comprises sucralose. In certain embodiments, the composition further comprises sucralose as about 0.01-0.25% based on the dry weight of all the components of the composition.

In certain embodiments, a provided composition further comprises a flavoring agent. In certain embodiments, the selection of a suitable flavoring agent to be added depends on the original taste sensation of the composition, including metallic, acidic, alkaline, salty, sweet, bitter and sour taste sensation. Certain flavoring agents, alone or in combination, mask specific taste sensations. For example, metallic taste could be masked with, but not limited to, flavoring agents based on berry fruits, grape, peppermint. For example, acidic taste could be masked with, but not limited to, flavoring agents based on lemon, lime, grapefruit, orange, cherry and/or strawberry. For example, alkaline taste could be masked with, but not limited to, flavoring agents based on aniseed, caramel, passion fruit, peach and/or banana. For example, salty taste could be masked with, but not limited to, flavoring agents based on butterscotch, caramel, hazelnut, spicy, maple, apricot, apple, peach, vanilla and/or wintergreen mint. For example, bitter taste could be masked with, but not limited to, flavoring agents based on licorice, passion fruit, coffee, chocolate, peppermint, grapefruit, cherry, peach, raspberry, wild cherry, walnut, mint and/or anise. For example, sweet taste could be masked with, but not limited to, flavoring agents based on grape, cream, caramel, banana, vanilla and/or fruit berry. For example, sour taste could be masked with, but not limited to, flavoring agents based on citrus flavors, licorice, root, bear and/or raspberry. Flavoring agents can be used alone or in combination and its selection will be dependent also upon the target population and any other substance (e.g., a pharmaceutical agent) incorporated on the composition. The perception of the flavoring agent changes from individual to individual and also with age: typically a geriatric population will prefer mint or orange flavors whereas younger populations tend to prefer flavors like fruit punch, raspberry, etc. Generally, the amount of flavoring agent needed to mask an unpleasant taste or improve taste overall will depend not only on the composition of the formulation but also on the flavor type and its strength.

In certain embodiments, a flavoring agent is a palatable flavor that has a long shelf life and which does not crystallize or precipitate out of the composition upon storage. In certain embodiments, flavoring agents may be natural flavors, derived from various parts of the plants like leaves, fruits and flowers, or synthetic flavor oils or powders. Exemplary flavor oils that may be used in or as flavoring agents include, but are not limited to, peppermint oil, cinnamon oil, spearmint oil, and oil of nutmeg. Exemplary fruity flavors that may be used in or as flavoring agents include, but are not limited to, vanilla, cocoa, coffee, chocolate and citrus. Exemplary fruit essence flavors that may be used in or as flavoring agents include, but are not limited to, apple, raspberry, cherry, and pineapple. The amount of flavoring agent added can vary with the flavor employed. In some embodiments, the concentration of the flavoring agent in the composition is between about 0% and 5%, by weight. In some embodiments, the concentration of the flavoring agent in the composition is between 0.001% and 5%, inclusive, by weight. In some embodiments, the concentration of the flavoring agent in the composition is between 0.1% and 1%, inclusive, by weight. In some embodiments, the concentration of the flavoring agent in the composition is between 0.5% and 1%, inclusive, by weight.

Cooling agents may also be added in order to improve the after-taste of mucoadhesive composition. Exemplary cooling agents include, but are not limited to, neohesperidine dihydrochalcone, menthol flavor, L-Menthol and some polyol sugars which are widely used for this purpose. Other components can also be added that should compete with sensory stimuli, such as Cremophor (which is used to coat the surface protein receptors), or saline solutions (e.g. sodium chloride, which competes within channel receptors with the bitter stimuli to reduce the overall perception of bitterness). In certain embodiments, the cooling agents in the composition is one or a combination of neohesperidine dihydrochalcone, menthol and/or polyol sugar. In certain embodiments, the mucoadhesive composition further comprises cooling agents of about 0% to about 5% based on the weight of all the components of the composition. In certain embodiments, the mucoadhesive composition further comprises cooling agents as about 0.001% to about 2.5% based on the weight of all the components of the composition.

In certain embodiments, a provided composition further comprises a colorant. A colorant can be added to enhance the aesthetic appeal of the composition, especially when formulation ingredients or drugs are presented in a non-solution form. Generally, any colorant could be added, such as for example FD&C pigments (for example blue no. 1, blue no. 2, red no. 3, red no. 40, yellow no. 5, or yellow no. 6). Exemplary colorants include, but are not limited to annatto extract, dehydrated beets (beet powder), canthaxanthin, caramel, β-apo-8′-carotenal, β-carotene, cochineal extract, carmine, sodium copper chlorophyllin, toasted partially defatted cooked cottonseed flour, ferrous gluconate, ferrous lactate, grape color extract, grape skin extract (enocianina), synthetic iron oxide, fruit juice, vegetable juice, carrot oil, paprika, paprika oleoresin, mica-based pearlescent pigments, riboflavin, saffron, spirulina extract, titanium dioxide, tomato lycopene extract; tomato lycopene concentrate, turmeric, turmeric oleoresin, alumina (dried aluminum hydroxide), calcium carbonate, potassium sodium copper chlorophyllin (chlorophyllin-copper complex), dihydroxyacetone, bismuth oxychloride, synthetic iron oxide, ferric ammonium ferrocyanide, ferric ferrocyanide, chromium hydroxide green, chromium oxide greens, guanine, pyrophyllite, mica, talc, aluminum powder, bronze powder, copper powder, zinc oxide, bismuth citrate, disodium EDTA-copper, guaiazulene, henna, lead acetate, pyrophyllite, silver, ultramarines, manganese violet, luminescent zinc sulfide, FD&C Blue No. 1, FD&C Blue No. 2, FD&C Green No. 3, Orange B, Citrus Red No. 2, FD&C Red No. 3, FD&C Red No. 40, FD&C Yellow No. 5, FD&C Yellow No. 6, D&C Blue No. 4, D&C Green No. 5, D&C Green No. 6, D&C Green No. 8, D&C Orange No. 4, D&C Orange No. 5, D&C Orange No. 10, D&C Orange No. 11, FD&C Red No. 4, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Red No. 39, D&C Violet No. 2, D&C Yellow No. 7, Ext. D&C Yellow No. 7, D&C Yellow No. 8, D&C Yellow No. 10, D&C Yellow No. 11, D&C Black No. 2, D&C Black No. 3, D&C Brown No. 1, and Ext. D&C Violet No. 2. In certain embodiments, a colorant represents 0.001% to about 0.5% based on the weight of all the components of the composition. In some embodiments, the concentration of the colorant in the composition is between 0.001% and 5%, inclusive, by weight. In some embodiments, the concentration of the colorant in the composition is between 0.001% and 1%, inclusive, by weight.

In certain embodiments, a provided composition further comprises a surfactant. Exemplary surfactants include, but are not limited to, sorbitan fatty acid esters (e.g., sorbitan monoisostearate, sorbitan monolaurate, sorbitan monooleate, sorbitan monopalmitate, sorbitan monostearate, sorbitan sesquistearate, sorbitan sesquioleate, sorbitan trilaurate, sorbitan trioleate, sorbitan tristearate), sucrose palmitate, docusate sodium, glyceryl monooleate, vitamin E polyethylene glycol succinate, polyethylene glycol derivatives, polypropylene glycol derivatives, propylene glycol monolaurate, myristyl alcohol, polyoxyethylene derivatives, povidone, copovidone, polaxamer, polyoxyethylene alkyl ethers, polyoxyethylene castor oil derivatives, polyoxyl castor oil, sodium lauryl sulfate, and propylene glycol dilaurate. The amount of surfactant added can vary with the surfactant employed and active agent.

In certain embodiments, a provided composition further comprises one or more preservatives. The preservative employed in the invention can be any preservative, as long as does not negate other desirable properties of the composition. Example of a preservative is an antimicrobial preservative that is used to prevent or inhibit the growth of micro-organisms in the composition. Exemplary preservative agents include, but are not limited to, C3-C8 alcohols, phenylethyl alcohol, chlorbutanol, p-hydroxybenzoic, acid esters, benzathonium chloride and benzalkonium chloride, benzoic acid, propyl galate, methylparaben, propylparaben, sorbic acid, sodium benzoate and/or potassium sorbate. The amount of preservative agent added can vary with the preservative agent employed. In certain embodiments, a preservative agent represents about 0% to about 45% based on the weight of all the components of the composition. In certain embodiments, a preservative agent represents about 0% to about 1% (e.g., 0.025% to 0.2%) based on the weight of all the components of the composition.

In certain embodiments, a provided composition further comprises stabilizing agents such as antioxidants. Oxidative degradation of a composition could be accelerated by light and by the presence of mineral or metallic impurities due to the formation of free radicals. Antioxidants prevents the oxidation of active agents and/or excipients of the composition. Antioxidants could be classified as true antioxidants, as reducing agents and as antioxidant synergists. Exemplary antioxidants agents include, but are not limited to, Butylated hydroxytoluene (BHT), Butylated hydroxyanisole (BHA), Ascorbic acid, sodium bisulfate, sodium ascorbate, sodium edetate and chelating agents such as EDTA and or cyclodextrins. The amount of antioxidant agent added can vary with the antioxidant agent employed.

In certain embodiments, a provided composition further comprises penetration enhancement additives. Penetration enhancers effectively increase permeability of active agents and/or composition excipients. Preferably, penetration enhancement additives are compatible with the active agents and other formulation excipients, pharmacologically inert, nontoxic and inexpensive. Exemplary penetration enhancement additives include, but are not limited to, bile salts, surfactants, fatty acids and derivatives, glycerides, chelators, salicylates, polymers, or other compounds. Exemplary of bile salts that act as Penetration enhancement additives include, but are not limited to, Sodium glycocholate, sodium deoxycholate, sodium taurocholate, sodium fusidate, sodium glycodeoxycholate, sodium taurodihydrofusidate. Exemplary of surfactants that act as penetration enhancement additives include, but are not limited to, sodium lauryl sulfate, brij1-35, lysophosphatidylcholine, dioctyl sodium sulfosuccinate, laurenth-9, polysorbate-80, polyethyleneglycol-8-laurate, glyceryl monolaurate. Exemplary of fatty acids and derivatives that act as Penetration enhancement additives include, but are not limited to, sorbitan laurate, sodium caprate, sucrose palmitate, lauroyl choline, sodium myristate, palmitoyl carnitine. Exemplary of glycerides that act as penetration enhancement additives include, but are not limited to phospholipids, monohexanoin, medium chain glycerides. Exemplary of chelators that act as penetration enhancement additives include, but are not limited to ethylene diamine tetraacetate (EDTA), disodium EDTA. Exemplary of salicylates that act as penetration enhancement additives include, but are not limited to salicylic acid, sodium methoxysalicylate, acethyl slicylic acid. Exemplary of polymers that act as penetration enhancement additives include, but are not limited to chitosan, polycarbophil, sodium carboxymethylcellulose and their derivatives. Exemplary of other compounds that act as Penetration enhancement additives include, but are not limited to cyclodextrins, benzalkonium chloride, phenothiazines, nitric acid donors, menthol, zonula occluden toxin, poly-1-arginines, soybean derivative glucosides, citicholine, α-acid derivatives. The amount of penetration enhancement additives added can vary with the Penetration enhancement additives agent employed.

In certain embodiments, a provided composition further comprises preferably: one, both, or a mixture of hydroxypropyl cellulose M and a hydroxypropyl cellulose G as both a film forming and mucoadhesive polymer. Wherein, the concentration of the hydroxypropyl cellulose M in the liquid formulation is between 0.1% and 3% (e.g., 0.3%-1.8%) by weight; the concentration of the hydroxypropyl cellulose Gin the liquid formulation is between 0.1% and 3% (e.g., 0.6%-1.8%) by weight. Wherein, the concentration of the hydroxypropyl cellulose M in the liquid formulation is between 0.3% and 4% (e.g., 0.3%-1.8%) by weight; the concentration of the hydroxypropyl cellulose Gin the liquid formulation is between 0.19% and 6.5% (e.g., 0.6%4.8%) by weight. A provided composition further comprises polyvinylpyrrolidone copolymer as a film forming and mucoadhesive polymer, wherein, the concentration of the polyvinylpyrrolidone copolymer is about 0% to about 4% (e.g., 0%-2%) by weight or about 0% to about 2%. The composition further comprises about 0% to about 7% of one or a combination of Glycerol (e.g., at 1.5%-3.5%) and/or PEG-400 (e.g., at 0.6%4.8%) as a plasticizer; optionally cellulose derivative HPC (e.g., at 0-1.84%); optionally cellulose derivative MC (e.g., at 0-1.8%); about 10% to about 40% (e.g., at 33% to 40%, at 36% to 40%) by weight of Ethanol as an organic solvent; about 10% to about 65% (e.g., at 50% to 58%, at 51% to 58%) by weight of water as an inorganic solvent; about 0%-2% by weight of one or combination of pH adjusting agents (e.g., citric acid; e.g., at 0.1%-1.7%), wherein the total percentage by weight of all components does not exceed 100%. These proportions are without taking into account the active agent to be included in the formulation.

In certain embodiments, a provided composition further comprises about 0.1% to about 10% (e.g., 0.2%-0.9%) by weight of polyacrylic acid (e.g., a Carbopol®) as a film forming and mucoadhesive polymer. The provided composition optionally further comprises polyethylene glycol (e.g., PEG400) as a mucoadhesive polymer, wherein, the concentration of the polyethylene glycol is about 0% to about 76% (e.g., 0.75%-76%, 1.5%-76%, 1.5%-75%) by weight. The composition further comprises cellulose derivative as a mucoadhesive polymer, wherein, the concentration of the hydroxypropyl cellulose (HPC) derivative is about 0% to about 3% by weight. In certain embodiments, the composition further comprises cellulose derivative (HPC) (e.g., at 0%-1.8%). In certain embodiments, the composition further comprises cellulose derivative (MC) (e.g., at 0%-1.8% (e.g., 0.03%-1.8%)). The composition further comprises Glycerol as a plasticizer, with concentration ranging from about 0% to about 5% (e.g., 0%-3% (e.g., 0.9%-3%)). In certain embodiments, the composition is substantially free of a PEG and does not comprise both PVP-VA-64 and Carbopol®. The composition further comprises about 0% to about 40% (e.g., 12%-35%) by weight of ethanol as an organic solvent and about 10% to about 63% (e.g., 10.5%-58%) by weight of water as an inorganic solvent; about 0% to about 2% by weight of one or combination of pH adjusting agents, wherein the total percentage by weight of all components does not exceed 100%. These proportions are without taking into account the active agents to be included in the formulation.

In certain embodiments, a provided composition further comprises polyvinylpyrrolidone copolymer (e.g., PVP-VA-64) and polyethylene glycol as a film forming and mucoadhesive polymer. Wherein polyvinylpyrrolidone copolymer is about 0.1% to 10% or 0.7% to 10% (e.g., 7%-10%) by weight and polyethylene glycol (e.g., PEG400) is about 1.85% to about 77% (e.g., 40%-65%) by weight. The provided composition further comprises about 0% to about 5% (e.g., 1.5% to 3%) of glycerol as a plasticizer; about 4% to about 40% (e.g., 15%-20%) by weight of ethanol as an organic solvent; about 5% to about 60% (e.g., 10%-40%) by weight of water as an inorganic solvent; about 0% to about 2% by weight of one or a combination of pH adjusting agents (e.g., citric acid (e.g., at 0-0.12%)); optionally one or more cellulose derivatives (e.g., HPC (individual or mixture) (e.g., at 1%-1.5%); optionally a flavoring agent (e.g., Mint's or a derivative thereof) (e.g., at 0-2.5%); and optionally a sweetener (e.g., sucralose (e.g., at 0.01%-0.03%); wherein the total percentage by weight of all components does not exceed 100%. These proportions are without taking into account the active agents to be included in the formulation.

In certain embodiments, the compositions described herein further comprise a propellant, wherein the concentration of the propellant in the composition is between 0.1% and 10%, inclusive, by weight. In certain embodiments, the compositions described herein is substantially free of a propellant.

In certain embodiments, the compositions are substantially free of a propellant.

It will be understood that when a range is recited in the application, the end of the range are specifically disclosed as if specifically recited. For example, a range of about 19% to about 99% specifically include a disclosure separately of 19% and separately of 99%.

In certain embodiments, the active agent is included in an amount from about 0.001% to 50% based on the weight of all the components of the composition. In certain embodiments, the active agent is included in an amount from about 0.1% to 5% (e.g., 0.1% to 1%, 1% to 5%) based on the weight of all the components of the composition (e.g., when the composition is in the form of a solution, gel, suspension, or emulsion). In certain embodiments, the active agent is included in an amount from about 5% to 20% based on the weight of all the components of the composition (e.g., when the composition is in the form of a solution, gel, suspension, or emulsion). In certain embodiments, the active agent is included in an amount from about 20% to 50% based on the weight of all the components of the composition (e.g., when the composition is in the form of a suspension).

In certain embodiments, a pharmaceutical agent is included in a provided composition described herein. In certain embodiments, the provided composition can be designed such that a pharmaceutical agent included therein has a local effect. In certain embodiments, the provided composition can be designed such that a pharmaceutical agent included therein is absorbed systemically by the mucosal surface. In certain embodiments, the provided composition can be designed such that a pharmaceutical agent included therein mimics the pharmacokinetics of a traditional dosage form (e.g., an oral composition), such as a marketed dosage form, such as a capsule, tablet, oral dispersible films, oral sprays. Non-limiting examples of pharmaceutical agents that may be included in a provided composition described herein include 5HT3 antagonists, selective serotonin receptor (5-HT) agonists, Ace inhibitors, alcohols, alkaloid narcotics, alkaloids, alpha-1-adrenergic receptor antagonists, amides, amino acid preparations, anabolic preparations, barbiturate acid derivatives, benzodiazepines and derivatives, bromides, beta-adrenergic antagonists, dopamine D1/D2 antagonists, H2 antagonists, mineralocorticoids, monoamine oxidase inhibitors, acne drugs, agents for virulent carcinoma, Alzheimer's disease medicines, analeptics, analgesics, anesthetics, antacids, CGRP receptor antagonists, antiallergic medications, antianginal drugs, anti-anxiety agents, anti-arrhythmias, anti-asthmatics, antibiotics, anti-cholesterolemics, anticoagulants, anticonvulsants, antidepressants, antidiabetic drugs, anti-diarrhea preparations, anti-emetics, anti-epileptics, antifungals, antihistamines, anti-hypertensive drugs, anti-inflammatory drugs, anti-inflammatory anodynes, anti-inflammatory enzymes, anti-inflammatory steroids, anti-lipid agents, anti-malarials, anti-maniacs, anti-migraines, anti-nauseants, anti-neoplasics, anti-obesity drugs, antiparasitic agents, anti-Parkinsonian agents, anti-periodontitis agents, antipodagrics, antipsychotics, anti-pyretics, including analgesic anti-pyretics, anti-rheumatic agents, antispasmodic agents, anti-stroke agents, anti-thrombotic agents, anti-thyroid preparations, anti-tumor drugs, antitussives, anti-ulcer agents, anti-uricemic drugs, antivirals, appetite stimulants, appetite suppressants, awakening agents, biological response modifiers, blood coagulation inhibitors, blood modifiers, blood pressure depressing agents, blood vein dilating agents, blood vessel protective agents, bone metabolism regulators, bronchodilators, carbamates, cardiac strengthening agents, cardiotonic drugs, cardiovascular agents, central nervous system stimulants, cerebral circulation agents, cerebral dilators, chemically therapeutic agents, chemotherapeutics, cytostatic, cytotoxic agents, narcotics, chloral derivatives, cholagogues, cholinesterase inhibitors, contraceptives, coronary dilators, cough curing agents, cough suppressants, cyclin-dependent kinase inhibitors, decongestants, dermatological agents, diabetic angina agents, digesting organ curing agents, diuretics, DNA and genetic modifying drugs, drugs for renal failure, drugs for treating gastric disorders, drugs which selectively modify CNS function, hormone replacement therapies, emetic agents, endometriosis management agents, enzymes, erectile dysfunction therapies, erythropoietic drugs, expectorants, fertility agents, gastrointestinal agents, glucocorticoids, steroids, hardening agents, hemostatic agents, homeopathic remedies, hormonal drugs, hormones, hyperglycemic agents, hypoglycemic agents, hypercalcemia and hypocalcemia management agents, hypnotics, hypolipidemic drugs, hypotensives, immunomodulators, immunosuppressives, intestinal regulators, ion exchange resins, laxatives, local anesthetic agents, local narcotic agents, lupus erythematosus agents, metabolism ameliorators, migraine treatments, miotic agents, motion sickness treatments, mucolytic agents, muscle relaxants, narcotic analgesics, narcotic antagonists, neuromuscular blocking agents, neuromuscular drugs, neuroprotective agents, non-cyclic ureides, nootropics, obesity management agents, ophthalmic agents, osteoporosis drugs, ovarian hormones, oxytocic agents, oxytocics, parasympatholytics, parasympathomimetics, pepsin inhibitors, peptides, peripheral vasodilators, peristaltic stimulants, piperidinediones, progestogens, prolactin inhibitors, prostaglandins, protease inhibitors, proton pump inhibitors, psychoneurotopic agents, psychopharmacological drugs, psychotherapeutic agents, pyschotropics, quinazolone derivatives, respiratory agents, respiratory stimulants, rhinitis drugs, sedatives, somnifacients, selective serotonin reuptake inhibitors, sexual hormones, skeletal muscle relaxants, sleeping inducers, smoking cessation agents, sore throat and mouth treatments, periodontal disease treatments, statins, stomachics, styptic agents, sympatholytics, systemic anti-infective agents, nonsystemic anti-infective agents, terine relaxants, thrombolytic agents, thyroid preparations, antithyroid preparations, thyroid hormones, tranquilizers, tranquilizer antipsychotics, treatments for acute radiation exposure, treatments for attention-deficit hyperactivity disorder, treatments for glaucoma, treatments for gout, treatments for Sjorgren's syndrome, tremor preparations, tyrosine kinase inhibitor, ulcer treatments, uricosuric agents, urinary tract agents, vaccines, vasoconstrictors, vasodilators, vasopressors, and veterinary drugs. For example, in some embodiments, a pharmaceutical agent included in the composition described herein is 2′-deoxycytidine 5′-monophosphate, 2′:3′-cyclic monophosphate, 2′-deoxyadenosine 5′5-triphosphate, 2′-deoxyadenosine 5′-monophosphate, 2′-deoxyguanosine 5′-monophosphate, 3′,5′-cyclic monophosphate, 5′-monophosphate, 8B/9A-substituted oestra-L 3,5(10)-triene, acetaminophen, acycloguanosine, adenosine 3′,5′-cyclic monophosphate, afatinib, alaproclate, alexidine, alfacalcidol, almotriptan, alprazolam, ambrisentan, ambroxol, ambroxol hydrochloride, amitriptyline hydrochloride, amlodipine, amobarbital, amphotericin B, apomorphine, aprepitant, aripipazole, ascorbic acid, asenapine, aspirin, atenolol, atomoxetine, ATP, avitriptan, azasetron, azathioprine, batanopride, benzalkonium chloride, benzocaine, benzonatate, beta-histine, betamethasone, betaxolol, bis-biguanide, bretazenil, bromazepam, brompheniramine maleate, buprenorphine, buprenorphine hydrochloride, caffeine, caramiphen edisylate, carbemazepime, carbinoxamine maleate, cetirizine, cetirizine hydrochloride, cetyl pyridium chloride, cetylpyridinium chloride, chlophedianol hydrochloride, chlordiazepoxide, chlorhexidine, chlorhexidine digluconate and tetracaine combination, chlorpheniramine, chlorpromazine, cimetidine, ciprofloxacin, citalopram, clebopride, clemastine fumarate, clonazepam, clonixine, clozapine, cobamamide, codeine, cyclobenzaprine, cyclophosphamide, cytidine 5′-monophosphate, dalfampridine, dasatinib, dazopride, D-chlorpheniramine maleate, dapoxetine, combination of dapoxetine and tadalafil, deferiprone, delmopinol, desloratadine, dexchlorpheniramine maleate, dexketoprofen, dextromethorphan, dextromethorphan hydrobromide, diazepam, diclofenac, diclofenac sodium, diclofenac potassium, dicyclomine hydrochloride, diflunisal, dimenhydrinate, diphenhydramine citrate, diphenhydramine hydrochloride, diphenylpyraline hydrochloride, dolasetron, domiphen bromide, domperidone, donepezil hydrochloride, doxylamine succinate, dronabinol, dutasteride, EDTA, eletriptan, enalapril, enzalutamide, enoxacin, erlotinib, estazolam, estradiol, etoricoxib, everolimus, exemestane, ezetimibe, eszopiclone, famotidine, fenoprofen calcium, fentanyl, finasteride, fingolimod, flumazenil, flurazepam, fluorides, fluoxetine, fluvoxamine, frovatriptan, galantamine, glatiramer acetate, gefitinib, growth hormone releasing peptide-2, glimepiride, granisetron, grepafloxacin, guaifenesin, guanosine 2′:3′-cyclic monophosphate, guanosine 2′-monophosphate, guanosine 3′,5′-cyclic monophosphate, guanosine 3′-monophosphate, guanosine 5′-monophosphate, haloperidol, hexetidine, hydrocodone, hydrocortisone, hydromorphone, ibuprofen, ibrutinib, imatinib, imipramine hydrochloride, indomethacin, inosine 5′-monophosphate, iodine, ipecac, isopropyl antipyrine, itasetron, ketoprofen, ketotifen, ketotifen fumarate, lansoprazole, lenalidomide, L-argenin, levobetaxolol hydrochloride, levodopa, levofloxacin, levorphanol, levosulpiride, levothyroxine, linaclotide, lisinopril, liothyronine, L-lysine, lomefloxacin, loperamide, loperamide hydrochloride, loratidine, lorazepam, lormetazepam, L-valine, meclizine, mecobalamin, mefanamic acid, melatonin, melatonin analog, meloxicam, memantine, mequitazine, methadone, methylphenidate, metoclopramide, metophon, metopimazine, montelukast sodium, morphine, morphine sulfate, inosine 5′-monophosphate, nabilone, nalidixic acid, nalorphine, naloxone, naloxone hydrochloride dehydrate, naltrexone, naproxen, naratriptan, neramexane, nicotine, nicotine analog, nicotinic acid, nifedipine, nilvadipine, nimesulide, nisin formulations, nitrazepam, nitroglycerin, N-tetradecyl-4-ethylpyridinium chloride, nystatin, octapinol, octenidine, olanzapine, omeprazole, ondansetron, ondansetron base, orbifloxacin, oseltamivir carboxylate, oxazolam, oxybutinine, oxycodone, oxymorphone, palonosetron, pancopride, paracetamol, paroxetine, pentobarbital, phenols, phenylephrine, phenylpropanolamine, picosulfate sodium, piroxicam, potassium iodide, pramipexole, prednisolone, prelanenant, prochlorperazine, progesterone, progestin, promethazine hydrochloride, pronase, propranolol, propiverine, propoxyphene, pseudoephredrine hydrochloride, pyrilamine maleate, quaternary ammonium salts, romasetron, ranitidine, rasigiline, remacemide, repaglinide, risperidone, rivaroxaban, rivastigmine, rivastigmine tartrate, rizatriptan, roflumilast, ropinirole, rosuvastatin, roxithromycin, salbutamol, salicylanilide, salivary gland hormone, sanguinarine, scopolamine, selegiline, serrapeptase, sertraline, sildenafil, sildenafil citrate, simethicone, sinvastatine, solifenacin, streptodornase, streptokinase, structural homolog of adenosine 5′ monophosphate, sulfonamide, sulpiride, sumatriptan, sunitinib, tadalafil, tapentadol, tecfidera, tegafur, teriflunomide, terpin hydrate, tetradecylpyridinium chloride, tetrahydrolipstatin, thalidomide, thiamazole, thiocolchicine derivatives, timidazole, tocopherol nicotinate, tolmetin sodium, topiramate, tramadol hydrochloride, triazolam, triclosan, trihexyphenidyl, trimetazidine, trimethobenzamide, tripelennamine citrate, tripolyphosphate sodium, triprolidine hydrochloride, tropisetron, umatriptan, uridine 5′-monophosphate, valproic acid, vardenafil, vardenafil hydrochloride, vareniclina, vinpocetine, xanthone, xycodone, zatosetron, zinc compounds, zinc histidine, zolmitriptan, or zolpidem. Combinations of pharmaceutical agents (e.g., those described herein) may also be used in a film-forming and mucoadhesive composition. In certain embodiments, the pharmaceutical agent is an oncologic drug. Exemplary oncologic drugs include abemaciclib, abiraterone acetate, abraxane (paclitaxel albumin-stabilized nanoparticle formulation), abvd, abve, abve-pc, ac, acalabrutinib, ac-t, actemra (tocilizumab), adcetris (brentuximab vedotin), ade, ado-trastuzumab emtansine, adriamycin (doxorubicin hydrochloride), afatinib dimaleate, afinitor (everolimus), akynzeo (netupitant and palonosetron hydrochloride), aldara (imiquimod), aldesleukin, alecensa (alectinib), alectinib, alemtuzumab, alimta (pemetrexed disodium), aliqopa (copanlisib hydrochloride), alkeran for injection (melphalan hydrochloride), alkeran tablets (melphalan), aloxi (palonosetron hydrochloride), alunbrig (brigatinib), ameluz (aminolevulinic acid), amifostine, aminolevulinic acid, anastrozole, apalutamide, aprepitant, aranesp (darbepoetin alfa), aredia (pamidronate disodium), arimidex (anastrozole), aromasin (exemestane), arranon (nelarabine), arsenic trioxide, arzerra (ofatumumab), asparaginase Erwinia chrysanthemi, atezolizumab, avastin (bevacizumab), avelumab, axicabtagene ciloleucel, axitinib, azacitidine, azedra (iobenguane i 131), bavencio (avelumab), beacopp, beleodaq (belinostat), belinostat, bendamustine hydrochloride, bendeka (bendamustine hydrochloride), bep, besponsa (inotuzumab ozogamicin), bevacizumab, bexarotene, bicalutamide, bicnu (carmustine), binimetinib, bleomycin, blinatumomab, blincyto (blinatumomab), bortezomib, bosulif (bosutinib), bosutinib, braftovi (encorafenib), brentuximab vedotin, brigatinib, bumel, busulfan, busulfex (busulfan), cabazitaxel, cabometyx (cabozantinib-s-malate), cabozantinib-s-malate, caf, calquence (acalabrutinib), campath (alemtuzumab), camptosar (irinotecan hydrochloride), capecitabine, capox, carac (fluorouracil—topical), carboplatin, carboplatin-taxol, carfilzomib, carmustine, carmustine implant, casodex (bicalutamide), cem, cemiplimab-rwlc, ceritinib, cerubidine (daunorubicin hydrochloride), cervarix (recombinant hpv bivalent vaccine), cetuximab, cev, chlorambucil, chlorambucil-prednisone, CHOP, cisplatin, cladribine, clofarabine, clolar (clofarabine), CMF, cobimetinib, cometriq (cabozantinib-s-malate), copanlisib hydrochloride, copdac, copiktra (duvelisib), COPP, copp-abv, cosmegen (dactinomycin), cotellic (cobimetinib), crizotinib, CVP, cyclophosphamide, cyramza (ramucirumab), cytarabine, cytarabine liposome, cytosar-u (cytarabine), dabrafenib, dacarbazine, dacogen (decitabine), dacomitinib, dactinomycin, daratumumab, darbepoetin alfa, darzalex (daratumumab), dasatinib, daunorubicin hydrochloride, daunorubicin hydrochloride and cytarabine liposome, decitabine, defibrotide sodium, defitelio (defibrotide sodium), degarelix, denileukin diftitox, denosumab, depocyt (cytarabine liposome), dexamethasone, dexrazoxane hydrochloride, dinutuximab, docetaxel, doxil (doxorubicin hydrochloride liposome), doxorubicin hydrochloride, doxorubicin hydrochloride liposome, dox-sl (doxorubicin hydrochloride liposome), durvalumab, duvelisib, efudex (fluorouracil—topical), eligard (leuprolide acetate), elitek (rasburicase), ellence (epirubicin hydrochloride), elotuzumab, eloxatin (oxaliplatin), eltrombopag olamine, emend (aprepitant), empliciti (elotuzumab), enasidenib mesylate, encorafenib, enzalutamide, epirubicin hydrochloride, epoch, epoetin alfa, epogen (epoetin alfa), erbitux (cetuximab), eribulin mesylate, erivedge (vismodegib), erleada (apalutamide), erlotinib hydrochloride, erwinaze (asparaginase Erwinia chrysanthemi), ethyol (amifostine), etopophos (etoposide phosphate), etoposide, etoposide phosphate, evacet (doxorubicin hydrochloride liposome), everolimus, evista (raloxifene hydrochloride), evomela (melphalan hydrochloride), exemestane, 5-FU (fluorouracil injection), 5-FU (fluorouracil—topical), fareston (toremifene), farydak (panobinostat), faslodex (fulvestrant), FEC, femara (letrozole), filgrastim, firmagon (degarelix), fludarabine phosphate, fluoroplex (fluorouracil—topical), fluorouracil injection, fluorouracil-topical, flutamide, folfiri, folfiri-bevacizumab, folfiri-cetuximab, folfirinox, folfox, folotyn (pralatrexate), fostamatinib disodium, FU-LV, fulvestrant, fusilev (leucovorin calcium), gardasil (recombinant hpv quadrivalent vaccine), gardasil 9 (recombinant hpv nonavalent vaccine), gazyva (obinutuzumab), gefitinib, gemcitabine hydrochloride, gemcitabine-cisplatin, gemcitabine-oxaliplatin, gemtuzumab ozogamicin, gemzar (gemcitabine hydrochloride), gilotrif (afatinib dimaleate), gleevec (imatinib mesylate), gliadel wafer (carmustine implant), glucarpidase, goserelin acetate, granisetron, granisetron hydrochloride, granix (filgrastim), halaven (eribulin mesylate), hemangeol (propranolol hydrochloride), herceptin (trastuzumab), HPV bivalent vaccine, recombinant, hpv nonavalent vaccine, recombinant, HPV quadrivalent vaccine, recombinant, hycamtin (topotecan hydrochloride), hydrea (hydroxyurea), hydroxyurea, hyper-cvad, ibrance (palbociclib), ibritumomab tiuxetan, ibrutinib, iclusig (ponatinib hydrochloride), idarubicin hydrochloride, idelalisib, idhifa (enasidenib mesylate), ifex (ifosfamide), ifosfamide, IL-2 (aldesleukin), imatinib mesylate, imbruvica (ibrutinib), imfinzi (durvalumab), imiquimod, imlygic (talimogene laherparepvec), inlyta (axitinib), inotuzumab ozogamicin, interferon alfa-2b recombinant, interleukin-2 (aldesleukin), intron A (recombinant interferon alfa-2b), iobenguane I 131, ipilimumab, iressa (gefitinib), irinotecan hydrochloride, irinotecan hydrochloride liposome, istodax (romidepsin), ivosidenib, ixabepilone, ixazomib citrate, ixempra (ixabepilone), jakafi (ruxolitinib phosphate), JEB, jevtana (cabazitaxel), kadcyla (ado-trastuzumab emtansine), kepivance (palifermin), keytruda (pembrolizumab), kisqali (rib ociclib), kymriah (ti sagenlecleucel), kyprolis (carfilzomib), lanreotide acetate, lapatinib ditosylate, larotrectinib sulfate, lartruvo (olaratumab), lenalidomide, lenvatinib mesylate, lenvima (lenvatinib mesylate), letrozole, leucovorin calcium, leukeran (chlorambucil), leuprolide acetate, levulan kerastik (aminolevulinic acid), libtayo (cemiplimab-rwlc), lipodox (doxorubicin hydrochloride liposome), lomustine, lonsurf (trifluridine and tipiracil hydrochloride), lorbrena (lorlatinib), lorlatinib, lumoxiti (moxetumomab pasudotox-tdfk), lupron (leuprolide acetate), lupron depot (leuprolide acetate), lutathera (lutetium Lu 177-dotatate), lutetium (Lu 177-dotatate), lynparza (olaparib), marqibo (vincristine sulfate liposome), matulane (procarbazine hydrochloride), mechlorethamine hydrochloride, megestrol acetate, mekinist (trametinib), mektovi (binimetinib), melphalan, melphalan hydrochloride, mercaptopurine, mesna, mesnex (mesna), methotrexate, methylnaltrexone bromide, midostaurin, mitomycin C, mitoxantrone hydrochloride, mogamulizumab-kpkc, moxetumomab pasudotox-tdfk, mozobil (plerixafor), mustargen (mechlorethamine hydrochloride), MVAC, myleran (busulfan), mylotarg (gemtuzumab ozogamicin), nanoparticle paclitaxel (paclitaxel albumin-stabilized nanoparticle formulation), navelbine (vinorelbine tartrate), necitumumab, nelarabine, neratinib maleate, nerlynx (neratinib maleate), netupitant and palonosetron hydrochloride, neulasta (pegfilgrastim), neupogen (filgrastim), nexavar (sorafenib tosylate), nilandron (nilutamide), nilotinib, nilutamide, ninlaro (ixazomib citrate), niraparib tosylate monohydrate, nivolumab, nplate (romiplostim), obinutuzumab, odomzo (sonidegib), oepa, ofatumumab, olaparib, olaratumab, omacetaxine mepesuccinate, oncaspar (pegaspargase), ondansetron hydrochloride, onivyde (irinotecan hydrochloride liposome), ontak (denileukin diftitox), opdivo (nivolumab), OPPA, osimertinib, oxaliplatin, paclitaxel, paclitaxel albumin-stabilized nanoparticle formulation, PAD, palbociclib, palifermin, palonosetron hydrochloride, palonosetron hydrochloride and netupitant, pamidronate disodium, panitumumab, panobinostat, pazopanib hydrochloride, PCV, PEB, pegaspargase, pegfilgrastim, peginterferon alfa-2B, peg-intron (peginterferon alfa-2b), pembrolizumab, pemetrexed disodium, perj eta (pertuzumab), pertuzumab, plerixafor, pomalidomide, pomalyst (pomalidomide), ponatinib hydrochloride, portrazza (necitumumab), poteligeo (mogamulizumab-kpkc), pralatrexate, prednisone, procarbazine hydrochloride, procrit (epoetin alfa), proleukin (aldesleukin), prolia (denosumab), promacta (eltrombopag olamine), propranolol hydrochloride, provenge (sipuleucel-t), purinethol (mercaptopurine), purixan (mercaptopurine), radium 223 dichloride, raloxifene hydrochloride, ramucirumab, rasburicase, ravulizumab-cwvz, R-CHOP, R-CVP, recombinant human papillomavirus (hpv) bivalent vaccine, recombinant human papillomavirus (HPV) nonavalent vaccine, recombinant human papillomavirus (HPV) quadrivalent vaccine, recombinant interferon alfa-2B, regorafenib, relistor (methylnaltrexone bromide), R-epoch, retacrit (epoetin alfa), revlimid (lenalidomide), rheumatrex (methotrexate), ribociclib, r-ice, rituxan (rituximab), rituxan hycela (rituximab and hyaluronidase human), rituximab, rituximab and hyaluronidase human, rolapitant hydrochloride, romidepsin, romiplostim, rubidomycin (daunorubicin hydrochloride), rubraca (rucaparib camsylate), rucaparib camsylate, ruxolitinib phosphate, rydapt (midostaurin), sancuso (granisetron), sclerosol intrapleural aerosol (talc), siltuximab, sipuleucel-t, somatuline depot (lanreotide acetate), sonidegib, sorafenib tosylate, sprycel (dasatinib), Standford V, sterile talc powder (talc), steritalc (talc), stivarga (regorafenib), sunitinib malate, sustol (granisetron), sutent (sunitinib malate), sylatron (peginterferon alfa-2b), sylvant (siltuximab), synribo (omacetaxine mepesuccinate), tabloid (thioguanine), tafinlar (dabrafenib), tagrisso (osimertinib), talc, talimogene laherparepvec, tamoxifen citrate, tarabine pfs (cytarabine), tarceva (erlotinib hydrochloride), targretin (bexarotene), tasigna (nilotinib), tavalisse (fostamatinib disodium), taxol (paclitaxel), taxotere (docetaxel), tecentriq (atezolizumab), temodar (temozolomide), temozolomide, temsirolimus, thalidomide, thalomid (thalidomide), thioguanine, thiotepa, tibsovo (ivosidenib), tisagenlecleucel, tocilizumab, tolak (fluorouracil—topical), topotecan hydrochloride, toremifene, torisel (temsirolimus), totect (dexrazoxane hydrochloride), TPF, trabectedin, trametinib, trastuzumab, treanda (bendamustine hydrochloride), trexall (methotrexate), trifluridine and tipiracil hydrochloride, trisenox (arsenic trioxide), tykerb (lapatinib ditosylate), ultomiris (ravulizumab-cwvz), unituxin (dinutuximab), uridine triacetate, vac, valrubicin, valstar (valrubicin), vandetanib, vamp, varubi (rolapitant hydrochloride), vectibix (panitumumab), veip, velcade (bortezomib), vemurafenib, venclexta (venetoclax), venetoclax, verzenio (abemaciclib), vidaza (azacitidine), vinblastine sulfate, vincristine sulfate, vincristine sulfate liposome, vinorelbine tartrate, VIP, vismodegib, vistogard (uridine triacetate), vitrakvi (larotrectinib sulfate), vizimpro (dacomitinib), voraxaze (glucarpidase), vorinostat, votrient (pazopanib hydrochloride), vyxeos (daunorubicin hydrochloride and cytarabine liposome), xalkori (crizotinib), xeloda (capecitabine), xeliri, xelox, xgeva (denosumab), xofigo (radium 223 dichloride), xtandi (enzalutamide), yervoy (ipilimumab), yescarta (axicabtagene ciloleucel), yondelis (trabectedin), zaltrap (ziv-aflibercept), zarxio (filgrastim), zejula (niraparib tosylate monohydrate), zelboraf (vemurafenib), zevalin (ibritumomab tiuxetan), zinecard (dexrazoxane hydrochloride), ziv-aflibercept, zofran (ondansetron hydrochloride), zoladex (goserelin acetate), zoledronic acid, zolinza (vorinostat), zometa (zoledronic acid), zydelig (idelalisib), zykadia (ceritinib), and zytiga (abiraterone acetate). In certain embodiments, the pharmaceutical agent is Tinibs. In some embodiments, the pharmaceutical agent is Ciclibs. In certain embodiments, the pharmaceutical agent is a direct factor Xa inhibitor. Exemplary direct factor Xa inhibitors include dabigatran, rivaroxaban (Xarelto), apixaban (Eliquis), betrixaban, pradaxa, fondaparinus, darexaban (YM150), edoxaban (Lixiana), otamixaban, letaxaban (TAK-442), and eribaxaban (PD0348292). In certain embodiments, the pharmaceutical agent is rivaroxaban. In certain embodiments, the pharmaceutical agent is used to treat erectile dysfunction. Exemplary pharmaceutical agents used to treat erectile dysfunction include Cialis, Viagra, sildenafil, Staxyn, Levitra, tadalafil, vardenafil, alprostadil, avanafil (PDE-5 inhibitor), alprostadil, acecarbromal, apomorphine, methyltestosterone, PF-00446687, PL-6983, prostaglandin E1, THIQ, and trimix. In some embodiments, the pharmacuetical agent is a sleep aid or substance used to improve sleep. Exemplary sleep aids include diphenhydramine, trazodone, Ambien, zolpidem, temazepam, Lunesta, Ativan, Restoril, amitriptyline, Ambien CR, lorazepam, Prosom, estazolam, Belsomra, suvorexant, clonazepam, doxepin, eszopiclone, Halcion, Rozerem, diphenhydramine mirtazapine, gabapentin, Silenor, Sonata, quetiapine, flurazepam, Intermezzo, estazola doxylamine, doxylamine, triazolam, Unisom, olanzapine, secobarbital, Seconal, zaleplon, suvorexant, diphenhydramine with ibuprofen, quazepam, ramelteon, Seconal Sodium, acetaminophen with diphenhydramine, diphenhydramine hydrochloride, Edluar, zolpidem tartrate, chloral hydrate, Compoz Nighttime Sleep Aid, Doral, Nytol, oxazepam pentobarbital, Sominex, tasimelteon, butabarbital, dimenhydrinate, Hetlioz, phenobarbital, Twilite, Z-Sleep, Aceta-Gesic, Aleve PM, amobarbital, aspirin with diphenhydramine, diphenhydramine with magnesium salicylate, diphenhydramine with naproxen, diphenhydramine with aspirin and acetaminophen, SleepTabs, Tylenol PM, Unisom SleepGels, Unisom SleepMelts, Zolpimist, ZzzQuil, Nytol QuickCaps, Simply Sleep Sleep Aid, Sleepinal, Tranquil Nighttime Sleep Aid, Bayer PM, Care One Sleep Aid, Doans PM, Equaline Sleep Aid, Equate Sleep Aid, Excedrin PM, Headache Relief PM, and Ibuprofen PM. In some embodiments, the pharmaceutical agent is used to treat a pediatric patient. Exemplary medications used to treat pediatric patients include abacavir, abacavir and lamivudine, abacavir with dolutegravir and lamivudine, abacavir with lamivudine and zidovudine, abatacept, abobotulinumtoxin A, acarbose, acetaminophen, acetaminophen and codeine, acetazolamide, acetic acid, propylene glycol diacetate, hydrocortisone, acetohydroxamic acid, acetylcysteine, acrivastine and pseudoephedrine, acyclovir, acyclovir and hydrocortisone, adalimumab, adapalene, adapalene and benzoyl peroxide, adefovir, adenosine, agalsidase beta, albendazole, albumin, albuterol, alcaftadine, alclometasone, ethyl alcohol, isopropyl alcohol, aldesleukin, alendronate, alfentanil, alglucosidase alfa, aliskiren, allopurinol, almotriptan, alprazolam, alprostadil, alteplase, aluminum acetate, aluminum hydroxide, aluminum hydroxide and magnesium hydroxide, amantadine, amifostine, amikacin, amikacin (systemic), amiloride, aminocaproic acid, aminophylline, amiodarone, amitriptyline, amlodipine, ammonium chloride, amobarbital, amoxicillin, amoxicillin and clavulanate, amphetamine, amphotericin B (conventional), amphotericin B (lipid complex), amphotericin B (liposomal), amphotericin b cholesteryl sulfate complex, ampicillin, ampicillin and sulbactam, amyl nitrite, anagrelide, anakinra, anthralin, anthrax immune globulin (human), anthrax vaccine adsorbed, antihemophilic factor (human), antihemophilic factor (recombinant), antihemophilic factor/von willebrand factor complex (human), anti-inhibitor coagulant complex (human), antithymocyte globulin (equine), antithymocyte globulin (rabbit), antivenin (Latrodectus mactans), aprepitant, argatroban, arginine, aripiprazole, arsenic trioxide, artemether and lumefantrine, artesunate, articaine and epinephrine, artificial tears, ascorbic acid, asenapine, asfotase alfa, asparaginase (Erwinia), aspirin, atazanavir, atenolol, atomoxetine, atorvastatin, atovaquone, atovaquone and proguanil, atracurium, atropine, atropine (ophthalmic), atropine (systemic), atropine and pralidoxime, auranofin, avelumab, azacitidine, azathioprine, azelaic acid, azelastine, azelastine (nasal), azelastine (ophthalmic), azelastine and fluticasone, azithromycin, azithromycin (ophthalmic), azithromycin (systemic), aztreonam, aztreonam (oral inhalation), aztreonam (systemic), bacitracin, bacitracin (ophthalmic), bacitracin (systemic), bacitracin (topical), bacitracin and polymyxin B, bacitracin and polymyxin B (ophthalmic), bacitracin and polymyxin B (topical), bacitracin with neomycin, and polymyxin B, bacitracin with neomycin, and polymyxin B (ophthalmic), bacitracin with neomycin and polymyxin B (topical), bacitracin with neomycin, polymyxin B, and hydrocortisone, bacitracin with neomycin, polymyxin B, and hydrocortisone (ophthalmic), bacitracin with neomycin, polymyxin B, and hydrocortisone (topical), bacitracin with neomycin, polymyxin B, and pramoxine, baclofen, balanced salt solution, baloxavir marboxil, balsalazide, barium, basiliximab, bcg vaccine (immunization), beclomethasone, beclomethasone (nasal), beclomethasone (oral inhalation), Belladonna and opium, benazepril, benralizumab, bentoquatam, benzocaine, benzoin, benzonatate, benzoyl peroxide, benztropine, benzyl alcohol, benzylpenicilloyl polylysine, beractant, besifloxacin, betaine, betamethasone, betamethasone (systemic), betamethasone (topical), betaxolol, betaxolol (ophthalmic), bethanechol, bevacizumab, biotin, bisacodyl, bismuth subgallate, bismuth sub salicylate, bleomycin, blinatumomab, bosentan, botulism antitoxin, heptavalent, botulism immune globulin (intravenous-human), brimonidine, brimonidine (ophthalmic), brinzolamide and brimonidine, brivaracetam, bromocriptine, brompheniramine and phenylephrine, brompheniramine and pseudoephedrine, brompheniramine with dextromethorphan and phenylephrine, brompheniramine with pseudoephedrine and dextromethorphan, budesonide, budesonide (nasal), budesonide (oral inhalation), budesonide (systemic), budesonide and formoterol, bumetanide, bupivacaine, buprenorphine, buprenorphine and naloxone, bupropion, burosumab-twza, buspirone, busulfan, butalbital and acetaminophen, butalbital with acetaminophen and caffeine, Cl inhibitor (human), caffeine, calamine, calaspargase pegol-mknl, calcipotriene and betamethasone, calcitonin, calcitriol, calcitriol (systemic), calcium acetate, calcium carbonate, calcium chloride, calcium citrate, calcium glubionate, calcium gluconate, calcium lactate, calfactant, canakinumab, candesartan, cannabidiol, capreomycin, capsaicin, captopril, carbamazepine, carbamide peroxide, carbinoxamine, carboplatin, carglumic acid, carisoprodol, carmustine, carvedilol, caspofungin, castor oil, cefaclor, cefadroxil, cefazolin, cefdinir, cefditoren, cefepime, cefixime, cefotaxime, cefotetan, cefoxitin, cefpodoxime, cefprozil, ceftaroline fosamil, ceftazidime, ceftibuten, ceftriaxone, cefuroxime, celecoxib, cenegermin-bkbj, centruroides immune F(ab′)2 (Equine), cephalexin, cerliponase alfa, cetirizine, cetirizine (systemic), charcoal, activated, chenodiol, chloral hydrate, chlorambucil, chloramphenicol (systemic), chlordiazepoxide, chlorhexidine gluconate, chlorhexidine gluconate (oral), chlorhexidine gluconate (topical), chlorophyll, chloroprocaine, chloroquine, chlorothiazide, chlorpheniramine, chlorpromazine, chlorthalidone, chlorzoxazone, cholecalciferol, cholestyramine resin, cholic acid, choline magnesium trisalicylate, chorionic gonadotropin (human), ciclesonide, ciclesonide (nasal), ciclesonide (oral inhalation), ciclopirox, cidofovir, cimetidine, ciprofloxacin, ciprofloxacin (ophthalmic), ciprofloxacin (otic), ciprofloxacin (systemic), ciprofloxacin and dexamethasone, ciprofloxacin and fluocinolone, ciprofloxacin and hydrocortisone, cisapride, cisatracurium, cisplatin, citalopram, citric acid, sodium citrate, potassium citrate, cladribine, clarithromycin, clemastine, clindamycin, clindamycin (systemic), clindamycin (topical), clindamycin and benzoyl peroxide, clindamycin and tretinoin, clobazam, clobetasol, clofarabine, clofazimine, clomipramine, clonazepam, clonidine, clopidogrel, clorazepate, clotrimazole, clotrimazole (oral), clotrimazole (topical), clozapine, coal tar, cocaine (topical), codeine, colchicine, colesevelam, colestipol, colistimethate, copper, corticotropin, cortisone, cosyntropin, crisaborole, cromolyn, cromolyn (nasal), cromolyn (ophthalmic), cromolyn (oral inhalation), cromolyn (systemic), crotalidae immune F(ab′)2 (equine), crotalidae polyvalent immune fab (ovine), crotamiton, cyanocobalamin, cyclobenzaprine, cyclopentolate, cyclopentolate and phenylephrine, cyclophosphamide, cycloserine, cyclosporine, cyclosporine (ophthalmic), cyclosporine (systemic), cyproheptadine, cysteamine, cysteamine (systemic), cysteine, cytarabine (conventional), cytomegalovirus immune globulin (intravenous-human), dacarbazine, daclizumab, dactinomycin, dalteparin, danazol, dantrolene, dapsone, dapsone (systemic), dapsone (topical), daptomycin, darbepoetin alfa, darunavir, daunorubicin (conventional), deferasirox, deferoxamine, defibrotide, deflazacort, demeclocycline, denosumab, desipramine, desloratadine, desmopressin, desoximetasone, dexamethasone, dexamethasone (ophthalmic), dexamethasone (systemic), dexchlorpheniramine, dexmedetomidine, dexmethylphenidate, dexrazoxane, dextran, dextroamphetamine, dextroamphetamine and amphetamine, dextromethorphan, dextrose, dextrose and ringer's injection (lactated), dextrose and sodium chloride, diazepam, diazoxide, dibucaine, diclofenac, diclofenac (systemic), dicloxacillin, dicyclomine, didanosine, difluprednate, digoxin, digoxin immune fab, dihydroergotamine, diltiazem, dimenhydrinate, dimercaprol, dimethyl sulfoxide, dinutuximab, diphenhydramine, diphenhydramine (systemic), diphenhydramine (topical), diphenoxylate and atropine, diphtheria and tetanus toxoids, diphtheria with tetanus toxoids, acellular pertussis, and poliovirus vaccine, diphtheria with tetanus toxoids, acellular pertussis, poliovirus and Haemophilus B conjugate vaccine, diphtheria with tetanus toxoids, acellular pertussis vaccine, diphtheria, tetanus toxoids, acellular pertussis, hepatitis B (recombinant), and poliovirus (inactivated) vaccine, dipyridamole, disopyramide, dobutamine, docetaxel, docusate, docusate and Senna, dolasetron, dolutegravir, dopamine, dornase alfa, dorzolamide, doxapram, doxazosin, doxepin, doxepin (systemic), doxorubicin (conventional), doxycycline, doxylamine, dronabinol, droperidol, dyclonine, ecallantide, echothiophate iodide, econazole, eculizumab, edetate calcium disodium, edrophonium, efavirenz, efavirenz with emtricitabine and tenofovir disoproxil fumarate, efavirenz with lamivudine and tenofovir disoproxil fumarate, eflornithine, elapegademase-LVLR, elosulfase alfa, eltrombopag, elvitegravir with cobicistat, emtricitabine and tenofovir alafenamide, elvitegravir with cobicistat, emtricitabine, and tenofovir disoproxil fumarate, emapalumab-lzsg, emicizumab-kxwh, emtricitabine, emtricitabine and tenofovir alafenamide, emtricitabine and tenofovir disoproxil fumarate, emtricitabine with rilpivirine and tenofovir alafenamide, emtricitabine with rilpivirine and tenofovir disoproxil fumarate, enalapril, enalaprilat, enfuvirtide, enoxaparin, entecavir, ephedrine, ephedrine (systemic), epinastine, epinephrine, epinephrine (nasal), epinephrine (oral inhalation), epinephrine (systemic), epoetin alfa, epoprostenol, ergocalciferol, ergotamine, ergotamine and caffeine, ertapenem, erythromycin, erythromycin (ophthalmic), erythromycin (systemic), erythromycin (topical), escitalopram, eslicarbazepine, esmolol, esomeprazole, estradiol, estradiol (systemic), estrogens (conjugated/equine), estrogens (conjugated/equine, systemic), estrogens (conjugated/equine, topical), etanercept, eteplirsen, ethacrynic acid, ethambutol, ethiodized oil, ethionamide, ethosuximide, etidronate, etodolac, etomidate, etoposide, etravirine, everolimus, ezetimibe, ezetimibe and simvastatin, factor IX (human), factor IX (recombinant), factor IX complex (human) [(factors II, IX, X)], factor VIIA (recombinant), factor XIII concentrate (human), famciclovir, famotidine, fat emulsion (fish oil and plant based), fat emulsion (fish oil based), fat emulsion (plant based), felbamate, felodipine, fenoldopam, fentanyl, ferric gluconate, ferrous fumarate, ferrous gluconate, ferrous sulfate, fexofenadine, fibrin sealant, fibrinogen concentrate (human), filgrastim, fingolimod, flecainide, fluconazole, flucytosine, fludarabine, fludrocortisone, flumazenil, flunisolide, flunisolide (nasal), flunisolide (oral inhalation), fluocinolone, fluocinolone (ophthalmic), fluocinolone (otic), fluocinolone (topical), fluocinonide, fluorescein, fluoride, fluoride varnishes, fluorometholone, fluorouracil, fluorouracil (systemic), fluoxetine, fluoxymesterone, flurandrenolide, flurazepam, fluticasone, fluticasone (nasal), fluticasone (oral inhalation), fluticasone (topical), fluticasone and salmeterol, fluvastatin, fluvoxamine, folic acid, fomepizole, formoterol, fosamprenavir, fosaprepitant, foscarnet, fosinopril, fosphenytoin, fulvestrant, furosemide, gabapentin, gadobenate dimeglumine, gadopentetate dimeglumine, gadoterate meglumine, gallium GA 68-dotatate, galsulfase, ganciclovir, ganciclovir (ophthalmic), ganciclovir (systemic), gatifloxacin, gemcitabine, gemtuzumab ozogamicin, gentamicin, gentamicin (ophthalmic), gentamicin (systemic), gentamicin (topical), gentian violet, glucagon, glucarpidase, glycerin, glycerol phenylbutyrate, glycopyrrolate, glycopyrrolate (systemic), glycopyrronium (topical), granisetron, grass pollen allergen extract (5 grass extract), grass pollen allergen extract (timothy grass), griseofulvin, guaifenesin, guaifenesin and codeine, guaifenesin and dextromethorphan, guanfacine, Haemophilus B conjugate vaccine, halobetasol, haloperidol, hemin, heparin, hepatitis A vaccine, hepatitis B immune globulin (human), hepatitis B vaccine (recombinant), hetastarch, histrelin, homatropine, hyaluronidase, hydralazine, hydrochlorothiazide, hydrochlorothiazide and spironolactone, hydrocodone and acetaminophen, hydrocodone and chlorpheniramine, hydrocodone and homatropine, hydrocortisone, hydrocortisone (systemic), hydrocortisone (topical), hydrogen peroxide, hydromorphone, hydroxocobalamin, hydroxychloroquine, hydroxyprogesterone caproate, hydroxyurea, hydroxyzine, hyoscyamine, hyoscyamine with atropine, scopolamine, and phenobarbital, ibuprofen, idarubicin, idursulfase, ifosfamide, iloprost, imatinib, imiglucerase, imipenem and cilastatin, imipramine, immune globulin, indigotindisulfonate sodium, indinavir, indomethacin, infliximab, influenza virus vaccine (inactivated), influenza virus vaccine (live/attenuated), influenza virus vaccine (recombinant), inhibitors and inducers of cytochrome P450 enzymes, inhibitors and inducers of p-glycoprotein, insulin aspart, insulin aspart protamine and insulin aspart, insulin degludec, insulin degludec and insulin aspart, insulin detemir, insulin glargine, insulin glulisine, insulin lispro, insulin lispro protamine and insulin lispro, insulin NPH, insulin NPH and insulin regular, insulin products, insulin regular, interferon alfa-2B, iobenguane I 131, iodine, iodixanol, iodoquinol, iohexol, ipilimumab, ipratropium, ipratropium (nasal), ipratropium (oral inhalation), irbesartan, irinotecan (conventional), iron dextran complex, iron sucrose, isoniazid, isoproterenol, isotretinoin (systemic), isradipine, itraconazole, ivacaftor, ivermectin, ivermectin (systemic), ivermectin (topical), Japanese encephalitis virus vaccine (inactivated), ketamine, ketoconazole, ketoconazole (systemic), ketoconazole (topical), ketorolac, ketorolac (ophthalmic), ketorolac (systemic), ketotifen, ketotifen (ophthalmic), labetalol, lacosamide, lactic acid and ammonium hydroxide, Lactobacillus, lactulose, lamivudine, lamivudine and tenofovir disoproxil fumarate, lamivudine and zidovudine, lamivudine, nevirapine, and zidovudine, lamotrigine, lanadelumab-flyo, lansoprazole, laronidase, larotrectinib, latanoprost, ledipasvir and sofosbuvir, letrozole, leucovorin calcium, leuprolide, levalbuterol, levetiracetam, levocarnitine, levocetirizine, levofloxacin, levofloxacin (ophthalmic), levofloxacin (systemic), levoleucovorin, levothyroxine, lidocaine, lidocaine (ophthalmic), lidocaine (systemic), lidocaine (topical), lidocaine and epinephrine, lidocaine and prilocaine, lidocaine and tetracaine, lifitegrast, lindane, linezolid, liothyronine, liotrix, lisdexamfetamine, lisinopril, lithium, lomustine, loperamide, lopinavir and ritonavir, loratadine, loratadine and pseudoephedrine, lorazepam, losartan, lovastatin, lucinactant, lumacaftor and ivacaftor, mafenide, magnesium carbonate, magnesium chloride, magnesium citrate, magnesium gluconate, magnesium hydroxide, magnesium L-aspartate hydrochloride, magnesium oxide, magnesium sulfate, malathion, management of drug extravasations, mannitol, mannitol (oral inhalation), mannitol (systemic), maraviroc, measles mumps and rubella virus vaccine, measles mumps rubella and varicella virus vaccine, mebendazole, mecasermin, mechlorethamine, mechlorethamine (systemic), meclizine, medium chain triglycerides, medroxyprogesterone, mefloquine, megestrol, meloxicam, melphalan, meningococcal (groups A/C/Y and W-135) diphtheria conjugate vaccine, meningococcal group B vaccine, meningococcal polysaccharide (groups C and Y) and Haemophilus B tetanus toxoid conjugate vaccine, meperidine, mepivacaine, mepolizumab, mercaptopurine, meropenem, mesalamine, mesna, metaproterenol, metformin, methadone, methamphetamine, methenamine, methimazole, methocarbamol, methohexital, methotrexate, methsuximide, methyldopa, methylene blue, methylphenidate, methylprednisolone, metoclopramide, metolazone, metoprolol, metreleptin, metronidazole, metronidazole (systemic), metronidazole (topical), metyrapone, mexiletine, micafungin, miconazole, miconazole (topical), midazolam, miglustat, milrinone, miltefosine, mineral oil, minocycline, minoxidil, minoxidil (systemic), mitotane, mitoxantrone, mivacurium, modafinil, molindone, mometasone, mometasone (nasal), mometasone (oral inhalation), mometasone (topical), mometasone and formoterol, montelukast, morphine (systemic), morphine sulfate, moxidectin, moxifloxacin, moxifloxacin (ophthalmic), moxifloxacin (systemic), mupirocin, mycophenolate, nabilone, nadolol, nafcillin, nalbuphine, naloxone, naphazoline, naphazoline (ophthalmic), naproxen, nedocromil (ophthalmic), nelarabine, nelfinavir, neomycin, neomycin and polymyxin B, neomycin with polymyxin B and dexamethasone, neomycin with polymyxin B and hydrocortisone, neomycin with polymyxin B and hydrocortisone (ophthalmic), neomycin with polymyxin B and hydrocortisone (otic), neomycin with polymyxin B and hydrocortisone (topical), neostigmine, nesiritide, nevirapine, niacin, nicardipine, nifedipine, nilotinib, nitazoxanide, nitisinone, nitric oxide, nitrofurantoin, nitroglycerin, nitroprusside, nivolumab, nizatidine, norepinephrine, norethindrone, nortriptyline, nusinersen, nystatin, nystatin (oral), nystatin (topical), obiltoxaximab, octreotide, ofloxacin, ofloxacin (ophthalmic), ofloxacin (otic), ofloxacin (systemic), olanzapine, olmesartan, omalizumab, omeprazole, omeprazole and sodium bicarbonate, onabotulinumtoxina, ondansetron, oprelvekin, oseltamivir, oxacillin, oxaliplatin, oxandrolone, oxaprozin, oxcarbazepine, oxybutynin, oxycodone, oxycodone and acetaminophen, oxycodone and aspirin, oxymetazoline, oxymetazoline (nasal), oxymetazoline (ophthalmic), oxymetholone, paliperidone, palivizumab, palonosetron, pamidronate, pancrelipase, pancuronium, pantoprazole, papaverine, papillomavirus (9-valent) vaccine (human, recombinant), papillomavirus (types 16, 18) vaccine (human, recombinant), papillomavirus (types 6, 11, 16, 18) vaccine (human, recombinant), paregoric, paricalcitol, paromomycin, paroxetine, pegademase bovine, pegaspargase, pegfilgrastim, peginterferon alfa-2A, peginterferon alfa-2B, pembrolizumab, penciclovir, penicillamine, penicillin G (parenteral/aqueous), penicillin G benzathine, penicillin G procaine, penicillin V potassium, pentamidine, pentamidine (oral inhalation), pentamidine (systemic), pentazocine, pentobarbital, pentostatin, pentoxifylline, peramivir, perampanel, permethrin, perphenazine, phenazopyridine, phenobarbital, phenoxybenzamine, phentolamine, phenylephrine, phenylephrine (nasal), phenylephrine (ophthalmic), phenylephrine (systemic), phenylephrine (topical), phenytoin, physostigmine, phytonadione, pilocarpine, pilocarpine (ophthalmic), pimecrolimus, pimozide, piperacillin and tazobactam, piroxicam (systemic), pneumococcal conjugate vaccine (13-valent), pneumococcal polysaccharide vaccine (23-valent), poliovirus vaccine (inactivated), polyethylene glycol 3350, polyethylene glycol-electrolyte solution, polymyxin b, polysaccharide-iron complex, poractant alfa, posaconazole, potassium acetate, potassium chloride, potassium citrate and citric acid, potassium gluconate, potassium iodide, potassium iodide and iodine, potassium phosphate, potassium phosphate and sodium phosphate, pralidoxime, pravastatin, praziquantel, prazosin, prednicarbate, prednisolone, prednisolone (ophthalmic), prednisolone (systemic), prednisone, pregabalin, primaquine, primidone, probenecid, procainamide, procarbazine, prochlorperazine, promethazine, promethazine and codeine, promethazine and phenylephrine, promethazine with phenylephrine and codeine, propantheline, proparacaine, propofol, propranolol, propylthiouracil, protamine, protein C concentrate (human), protriptyline, pseudoephedrine, pseudoephedrine and ibuprofen, Psyllium, pyrantel pamoate, pyrazinamide, pyridostigmine, pyridoxine, pyrimethamine, quetiapine, quinapril, quinidine, quinine, quinupristin and dalfopristin, rabeprazole, rabies immune globulin (human), rabies vaccine, raltegravir, ranitidine, rasburicase, raxibacumab, remifentanil, retapamulin, rho(D) immune globulin, ribavirin, ribavirin (oral inhalation), ribavirin (systemic), riboflavin, riboflavin 5′-phosphate, rifabutin, rifampin, rifapentine, rifaximin, rilonacept, rilpivirine, rimantadine, Ringer's injection (lactated), risperidone, ritonavir, rituximab, rizatriptan, rocuronium, ropivacaine, rosiglitazone, rosuvastatin, rotavirus vaccine, rufinamide, sacrosidase, salicylic acid, salmeterol, sapropterin, saquinavir, sarecycline, sargramostim, scopolamine, scopolamine (ophthalmic), scopolamine (systemic), sebelipase alfa, secobarbital, secretin, segesterone acetate and ethinyl estradiol, selenium, selenium sulfide, Senna, sertaconazole, sertraline, sevelamer, sildenafil, silver nitrate, silver sulfadiazine, simethicone, simvastatin, sirolimus, sodium acetate, sodium benzoate, sodium bicarbonate, sodium chloride, sodium citrate and citric acid, sodium glycerophosphate pentahydrate, sodium nitrite and sodium thiosulfate, sodium phenylacetate and sodium benzoate, sodium phenylbutyrate, sodium phosphates, sodium polystyrene sulfonate, sodium thiosulfate, sofosbuvir, somatropin, sorbitol, sotalol, spinosad, spironolactone, stavudine, streptomycin, succimer, succinylcholine, sucralfate, sucrose, sufentanil, sugammadex, sulfacetamide, sulfacetamide (ophthalmic), sulfacetamide (topical), sulfadiazine, sulfamethoxazole and trimethoprim, sulfasalazine, sulfur and salicylic acid, sulindac, sumatriptan, sumatriptan and naproxen, tacrolimus, tacrolimus (systemic), tacrolimus (topical), tafenoquine, taliglucerase alfa, tamoxifen, tamsulosin, tazarotene, technetium TC 99m sodium pertechnetate, tecovirimat, temozolomide, teniposide, tenofovir disoproxil fumarate, terazosin, terbinafine, terbinafine (systemic), terbinafine (topical), terbutaline, testosterone, tetanus immune globulin (human), tetanus toxoid (adsorbed), tetracaine, tetracaine (ophthalmic), tetracaine (systemic), tetracycline (systemic), tetrastarch, tezacaftor and ivacaftor, thalidomide, theophylline, thiamine, thioguanine, thioridazine, thiotepa, thiothixene, thrombin (topical), tiagabine, ticarcillin and clavulanate potassium, tigecycline, timolol, timolol (ophthalmic), tinidazole, tipranavir, ti sagenlecleucel, tizanidine, tobramycin, tobramycin (ophthalmic), tobramycin (oral inhalation), tobramycin (systemic), tocilizumab, tolmetin, tolnaftate, tolterodine, topical corticosteroids, topiramate, topotecan, torsemide, tramadol, tranexamic acid, travoprost, trazodone, tretinoin (systemic), tretinoin (topical), triamcinolone, triamcinolone (nasal), triamcinolone (ophthalmic), triamcinolone (systemic), triamcinolone (topical), triamterene, triazolam, trifluoperazine, trifluridine, trihexyphenidyl, trimethobenzamide, trimethoprim, trimethoprim and polymyxin B, triprolidine, triprolidine and pseudoephedrine, triptorelin, tromethamine, tropicamide, typhoid vaccine, undecylenic acid and derivatives, uridine triacetate, ursodiol, valacyclovir, valganciclovir, valproic acid and derivatives, valsartan, vancomycin, varicella virus vaccine, varicella-zoster immune globulin (human), vasopressin, vecuronium, velaglucerase alfa, venlafaxine, verapamil, vestronidase alfa-vjbk, vigabatrin, vinblastine, vincristine, vinorelbine, vitamin A, vitamin E, vitamin E (systemic), vitamin E (topical), voretigene neparvovec-rzyl, voriconazole, warfarin, yellow fever vaccine, zafirlukast, zanamivir, zidovudine, zileuton, zinc acetate, zinc chloride, zinc gluconate, zinc oxide, zinc sulfate, ziprasidone, zolmitriptan, zolpidem, and zonisamide. In some embodiments, the pharmaceutical agent is an immunosuppressant. exemplary immunosuppressant drugs include corticosteroids, prednisone (deltasone, orasone), budesonide (entocort ec), prednisolone (millipred), j anus kinase inhibitors, tofacitinib (xeljanz), calcineurin inhibitors, cyclosporine (neoral, sandimmune, sangcya), tacrolimus (astagraf xl, envarsus xr, prograf), mTOR inhibitors, sirolimus (rapamune), everolimus (afinitor, zortress), IMDH inhibitors, azathioprine (azasan, imuran), leflunomide (arava), mycophenolate (cellcept, myfortic), abatacept (orencia), adalimumab (humira), anakinra (kineret), certolizumab (cimzia), etanercept (enbrel), golimumab (simponi), infliximab (remicade), ixekizumab (taltz), natalizumab (tysabri), rituximab (rituxan), secukinumab (cosentyx), tocilizumab (actemra), ustekinumab (stelara), vedolizumab (entyvio), monoclonal antibodies, basiliximab (simulect), and daclizumab (zinbryta).

In certain embodiments, a nutraceutical agent or supplement is included in a provided composition described herein. In certain embodiments, the provided composition can be designed such that a nutraceutical agent or supplement included therein has a local effect. In certain embodiments, the provided composition can be designed such that a nutraceutical agent or supplement included therein is absorbed systemically by the mucosal surface. In certain embodiments, the provided composition can be designed such that a nutraceutical agent or supplement included therein is systemically absorbed by the oral mucosa. In certain embodiments, the provided composition can be designed such that a nutraceutical agent or supplement included therein mimics the pharmacokinetics of a traditional dosage form (e.g., an oral composition), such as a marketed dosage form, such as a capsule, tablet, oral dispersible films or oral sprays. Non-limiting examples of nutraceutical agents and supplements that may be included in the composition described herein include anesthetics, antibacterials, steroids, anticaries agents, anti-cavity ingredients, anti-gingivitis agents, anti-inflammatory agents, antioxidants, antiperspirants, antiplaque agents, antitussives, cold prevention agents, cold and allergy treatments, cough treatments, dermatological agents, diarrhea treatments, enzymes, erectile dysfunction treatments, female sexual dysfunction treatments, heartburn and dyspepsia agents, hemostatics, herbals, hydration agents, oral hygiene treatments, periodontal actives, periodontal disease drugs, pH control agents, plaque disclosing agents, pre-treatment and treatment for exposure to chemical weapons, provitamins, respiratory disorder treatments, sleep aids, smoking cessation, sore throat agents, stimulants, stomatitis therapies, tartar control agents, vaccines, vitamin derivatives, vitamin extracts, and vitamins. For example, in some embodiments, a nutraceutical agent or supplement included in the provided composition described herein is acerola, electrolytes, aloe, aluminium, amino acids, anise, antibiotics, antimicrobial essential oil, apple extract, arsenic, balsam pear, barium chlorite, benzocaine, beta-carotene, beta-glucans, bicarbonate, bioflavonoids, biotene (glucose oxidase lactose peroxidase and lysozyme), biotin, blueberry, boron, breath freshening agents, bromine, buckwheat, cadmium, calcium, calcium chlorite, calcium peroxide, carbohydrates, carbonate, carvacrol, catechol, cevimeline, chitosan, chlorides, chlorine, chlorine dioxide, choline, chromium, cinnamon, citral, cobalt, coenzyme Q10, copper, DHA, edible organic acid, EPA, erythritol, essential oils, eucalyptol, evening primrose, fluorine, folic acid, garlic, geraniol, germinated unpolished rice, ginkgo leaf, glucose tolerance factor chromium, glutathione, glycerin, grapefruit extracts, green tea, green tea extracts, guava sesame, herb extracts, herbs, hinokitiol, huperzine-A, hydrogen peroxide, hydrogen peroxide adduct of carbodiimide persulfate, hydroperoxide, inositol, iodine, iron, isomaltulose, kava-kava extract (e.g., standardized to 30% kavalactones), lactitol, lactobacillus, lithium, lithium chlorite, magnesium, magnesium chlorite, maltitol powdered hydrogenated glucose syrup, manganese, mannitol, manose, matrimony vine (e.g. lychium Chinese), melatonin, menthol, meswak extract, metal chlorite, methylsalicylate, minerals, molybdenum, molybdenum nickel, momordicae fructus, mugwort, mulberry leaf, niacin (vitamin B3), niacin amide, oils, organic peroxides, PABA, pantethine, pantothenic acid, papain, parched bean flour, peppermint, perborate salt, perboric acid, percarbonate salt, Perilla, peroxide generating compounds, peroxyacids, peroxycarbamate, persulfate salt, persulfates, phenol, phosphate ions, phosphorus, pilocarpine, polyalcohol, potassium, potassium chlorite, protein, PVP-hydrogen peroxide complex, pyridoxine (vitamin B6), red Ginseng extracts, riboflavin, rose hip, seaweed extracts, selenium, silicon, sodium, sodium chlorite, sorbitol, soybean isoflavone, spearmint, strontium, sugar, superoxide dismutase, sweet tea, tea tree oil, thiamine, thyme oil, thymol, tin, tree and plant components/extracts, turmeric, eucalyptol, vanadium, vanadium glutathionine, vitamin A, vitamin B complex, vitamin B1, vitamin B12, vitamin B2, vitamin B6, vitamin C, vitamin D, vitamin D3, vitamin E, vitamin K, vitamin P, vitamins, wintergreen, or zinc.

Other agents that may be included in the film-forming and mucoadhesive composition described herein include antidotes, antigens or allergens, recombinant allergens, allergoids, antimicrobial agents, antiperspirants, antiseptics, anti-smoking formula, aromatizing agents, botanicals, breath deodorizing agents, breath freshening agents, breath masking agents, Chinese medicines, comfort agents, conditioning agents, cosmetic agents, deodorant actives, diet formula, dyes, emollients, flavor masking agents, flavors, food products, fragrances, heating agents, humectants, insects, malodor control agents, minerals, moisturizers, mouthwash components, oral band, oral freshness formula, proteins, refreshment agents, saliva stimulating agents, sexual enhancement formula, sugars, veterinary agents, whitening agents, wound-burn protective agents, wound-healing drugs, and homeopathic medicines.

The amount of an active agent or combination of active agents thereof included in a provided composition described herein will depend on the indication target population. In some embodiments, a provided composition contains an effective amount of an active agent. The term “effective amount” as used herein, refers to a sufficient amount of the active agent to produce a desired outcome. The exact amount required will vary from subject to subject, depending on the species, age, and general condition of the subject, the indication and the particular agent. In some embodiments, when the active agent is a pharmaceutical agent or nutraceutical agent, the provided composition contains a therapeutically effective amount of the active agent. The term “therapeutically effective amount” as used herein refers to a sufficient amount of a pharmaceutical or nutraceutical agent to achieve the intended purpose, such as, for example, to cause a reduction of symptoms of a disease. In some embodiments, when the active agent is a pharmaceutical agent or nutraceutical agent, the provided composition contains a prophylactically effective amount of the active agent. A “prophylactically effective amount” refers to a sufficient amount of a pharmaceutical or nutraceutical agent to achieve the intended purpose, such as prevention of a disease, one or more symptoms associated with the disease, and/or the recurrence thereof. In certain embodiments, an effective amount of a composition is the effective amount of the active agent included in the composition.

It will be understood that the total daily usage of the composition described herein may be decided by an attending physician within the scope of sound medical judgment, and will depend safety and toxicity profile of the components of the composition. The specific therapeutically effective dose level for any particular patient or organism will depend upon a variety of factors including the disorder being treated and the severity of the disorder; the clinical studies results, the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts (see, for example, Goodman and Gilman's, “The Pharmacological Basis of Therapeutics”, Tenth Edition, A. Gilman, J. Hardman and L. Limbird, eds., McGraw-Hill Press, 155-173, 2001).

In certain embodiments, a composition described herein is as described in a table described herein, wherein the concentration of each component of the composition is independently between 90% and 110%, inclusive, of the concentration of the corresponding component in the table. In certain embodiments, a composition described herein is as described in a table described herein, wherein the concentration of each component of the composition is independently between 80% and 120%, inclusive, of the concentration of the corresponding component in the table. In certain embodiments, a composition described herein is as described in a table described herein, wherein the concentration of each component of the composition is independently between 70% and 130%, inclusive, of the concentration of the corresponding component in the table.

In certain embodiments, a mucoadhesive composition described herein has an adhesiveness force higher than adhesiveness force obtained with pre-hydrated gut (without composition). In certain embodiments, the difference between the adhesiveness force of a mucoadhesive composition described herein and pre-hydrated gut (without composition) is higher than 0 g. In certain embodiments, the difference between the adhesiveness force of a mucoadhesive composition described herein and pre-hydrated gut (without composition) is higher than 1 g. In certain embodiments, the difference between the adhesiveness force of a mucoadhesive composition described herein and pre-hydrated gut (without composition) is higher than 4 g. In certain embodiments, the difference between the Adhesiveness force of a mucoadhesive composition described herein and pre-hydrated gut (without composition) is higher than 10 g. In certain embodiments, the difference between the Adhesiveness force of a mucoadhesive composition described herein and pre-hydrated gut (without composition) is higher than 20 g.

In certain embodiments, a mucoadhesive composition described herein has a viscosity higher than about 1 mPas and lower than about 600000 mPas. In certain embodiments, a mucoadhesive composition described herein has a viscosity higher than about 1 mPas and lower than about 20000 mPas. In certain embodiments, a mucoadhesive composition described herein has a viscosity higher than about 1 mPas and lower than about 15000 mPas. In certain embodiments, a mucoadhesive composition described herein has a viscosity higher than about 1 mPas and lower than about 5000 mPas. In certain embodiments, a mucoadhesive composition described herein has a viscosity higher than about 1 mPas and lower than about 1500 mPas. In certain embodiments, a mucoadhesive composition described herein are dispensed by a pump-action spray and has a viscosity higher than about 1 mPas and lower than about 20000 mPas.

In certain embodiments, a composition described herein is mucoadhesive at different range of pH. In certain embodiments, a mucoadhesive composition described herein has a pH from about 1 to about 10. In certain embodiments, a mucoadhesive composition described herein preferably has a pH from about 2 to about 9.

In certain embodiments, a mucoadhesive composition described herein are dispensed using a pump-action spray. Pump-action sprays are characterized in requiring the application of external pressure for actuation, for example fast movement of index finger, mechanical or electrically initiated pressure. In certain embodiments, a mucoadhesive composition dispensed by pump-action spray required less than 10 kg of force to actuate pump-action spray. In certain embodiments, a mucoadhesive composition dispensed by pump-action spray required less than 8 kg of force to actuate pump-action spray. In certain embodiments, a mucoadhesive composition dispensed by pump-action spray required less than 6 kg of force to actuate pump-action spray.

Also encompassed by the present disclosure are kits (e.g., pharmaceutical packs). In certain embodiments, the kit comprises a pharmaceutical composition described herein, and instructions for using the pharmaceutical composition. In certain embodiments, the kit comprises a first container, wherein the first container includes the pharmaceutical composition. In some embodiments, the kit further comprises a second container. In certain embodiments, the second container includes an excipient (e.g., an excipient for dilution or suspension of the pharmaceutical composition). In certain embodiments, the second container includes an additional pharmaceutical agent. In some embodiments, the kit further comprises a third container. In certain embodiments, the third container includes an additional pharmaceutical agent. In some embodiments, the pharmaceutical composition included in the first container and the excipient or additional pharmaceutical agent included in the second container are combined to form one unit dosage form. In some embodiments, the pharmaceutical composition included in the first container, the excipient included in the second container, and the additional pharmaceutical agent included in the third container are combined to form one unit dosage form. In certain embodiments, each of the first, second, and third containers is independently a vial, ampule, bottle, syringe, dispenser package, tube, sprayer, or inhaler. In certain embodiments, at least one of the first, second, and third containers is a sprayer.

In certain embodiments, the instructions are for administering the pharmaceutical composition to a subject in need thereof. In certain embodiments, the instructions comprise information required by a regulatory agency, such as the U.S. Food and Drug Administration (FDA) or the European Agency for the Evaluation of Medicinal Products (EMA). In certain embodiments, the instructions comprise prescribing information.

### Methods of Use and Uses

The present disclosure also provides methods of using the compositions of the present disclosure. In another aspect, the present disclosure provides methods of delivering an active agent to a subject in need thereof comprising contacting a mucus of the subject in need thereof with an composition (e.g., an effective amount of the composition) (e.g., pharmaceutical composition, nutraceutical composition, cosmetic composition, supplementary composition) of the present disclosure.

In another aspect, the present disclosure provides methods of treating a disease in a subject in need thereof comprising contacting a mucus of the subject in need thereof with an effective amount (e.g., therapeutically effective amount) of an composition (e.g., pharmaceutical composition) of the present disclosure.

In another aspect, the present disclosure provides methods of preventing a disease in a subject in need thereof comprising contacting a mucus of the subject in need thereof with an effective amount (e.g., prophylactically effective amount) of an composition (e.g., pharmaceutical composition) of the present disclosure.

In another aspect, provided herein are uses of the compositions of the present disclosure in the manufacture of a medicament for use in a method (e.g., method of delivering an active agent to a subject in need thereof, method of treating a disease in a subject in need thereof, method of preventing a disease in a subject in need thereof) of the present disclosure.

In another aspect, provided herein are uses of the compositions of the present disclosure in a method (e.g., method of delivering an active agent to a subject in need thereof, method of treating a disease in a subject in need thereof, method of preventing a disease in a subject in need thereof) of the present disclosure.

In another aspect, provided herein are the compositions of the present disclosure for use in a method (e.g., method of delivering an active agent to a subject in need thereof, method of treating a disease in a subject in need thereof, method of preventing a disease in a subject in need thereof) of the present disclosure.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a dog. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice, transgenic pigs). In certain embodiments, a subject in need thereof is a subject in need of delivery of an active agent or a composition, a subject in need of treatment of a disease, or a subject in need of prevention of a disease.

In certain embodiments, the effective amount is effective in treating the disease. In certain embodiments, the effective amount is effective in preventing the disease. In certain embodiments, the disease (e.g., disease being treated or prevented using the pharmaceutical compositions of the present disclosure) is a genetic disease. The term “genetic disease” refers to a disease caused by one or more abnormalities in the genome of a subject, such as a disease that is present from birth of the subject. Genetic diseases may be heritable and may be passed down from the parents' genes. A genetic disease may also be caused by mutations or changes of the DNAs and/or RNAs of the subject. In such cases, the genetic disease will be heritable if it occurs in the germline. Exemplary genetic diseases include Aarskog-Scott syndrome, Aase syndrome, achondroplasia, acrodysostosis, addiction, adrenoleukodystrophy, albinism, ablepharon-macrostomia syndrome, alagille syndrome, alkaptonuria, alpha-1 antitrypsin deficiency, Alport's syndrome, Alzheimer's disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Friedreich's ataxia, Gaucher disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC), Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson's disease, paroxysmal nocturnal hemoglobinuria, Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma piginentosum, and Zellweger syndrome.

In certain embodiments, the disease is a proliferative disease. A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms”. An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis”, “metastatic”, or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CIVIL) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes post-surgical inflammation.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

In certain embodiments, the disease is a hematological disease. A “hematological disease” includes a disease which affects a hematopoietic cell or tissue. Hematological diseases include diseases associated with aberrant hematological content and/or function. Examples of hematological diseases include diseases resulting from bone marrow irradiation or chemotherapy treatments for cancer, diseases such as pernicious anemia, hemorrhagic anemia, hemolytic anemia, aplastic anemia, sickle cell anemia, sideroblastic anemia, anemia associated with chronic infections such as malaria, trypanosomiasis, HTV, hepatitis virus or other viruses, myelophthisic anemias caused by marrow deficiencies, renal failure resulting from anemia, anemia, polycythemia, infectious mononucleosis (EVI), acute non-lymphocytic leukemia (ANLL), acute myeloid leukemia (AML), acute promyelocytic leukemia (APL), acute myelomonocytic leukemia (AMMoL), polycythemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalassemia, antibody-mediated disorders such as transfusion reactions and erythroblastosis, mechanical trauma to red blood cells such as micro-angiopathic hemolytic anemias, thrombotic thrombocytopenic purpura and disseminated intravascular coagulation, infections by parasites such as Plasmodium, chemical injuries from, e.g., lead poisoning, and hypersplenism.

In certain embodiments, the disease is a neurological disease. The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including Alzheimer's disease, Parkinson's disease, amyotrophic lateral sclerosis, tauopathies (including frontotemporal dementia), and Huntington's disease. Examples of neurological diseases include headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; bbrain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

In certain embodiments, the disease is a painful condition. A “painful condition” includes neuropathic pain (e.g., peripheral neuropathic pain), central pain, deafferentation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawl symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g. nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition.

In certain embodiments, the disease is a psychiatric disorder. The term “psychiatric disorder” refers to a disease of the mind and includes diseases and disorders listed in the Diagnostic and Statistical Manual of Mental Disorders—Fourth Edition (DSM-IV), published by the American Psychiatric Association, Washington D. C. (1994). Psychiatric disorders include anxiety disorders (e.g., acute stress disorder agoraphobia, generalized anxiety disorder, obsessive-compulsive disorder, panic disorder, posttraumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, cannabis dependence, cocaine dependence, hallucinogen dependence, inhalant dependence, nicotine dependence, opioid dependence, phencyclidine dependence, and sedative dependence), adjustment disorder, autism, delirium, dementia, multi-infarct dementia, learning and memory disorders (e.g., amnesia and age-related memory loss), and Tourette's disorder.

In certain embodiments, the disease is a metabolic disorder. The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include the endocrine (hormonal) control system (e.g., the insulin pathway, the enteroendocrine hormones including GLP-1, PYY or the like), the neural control system (e.g., GLP-1 in the brain), or the like. Examples of metabolic disorders include diabetes (e.g., Type I diabetes, Type II diabetes, gestational diabetes), hyperglycemia, hyperinsulinemia, insulin resistance, and obesity.

In certain embodiments, the disease is a cardiovascular disease. In certain embodiments, the disease is a cardiovascular disease. In certain embodiments, the disease is atherogenesis or atherosclerosis. In certain embodiments, the disease is arterial stent occlusion, heart failure (e.g., congestive heart failure), a coronary arterial disease, myocarditis, pericarditis, a cardiac valvular disease, stenosis, restenosis, in-stent-stenosis, angina pectoris, myocardial infarction, acute coronary syndromes, coronary artery bypass grafting, a cardio-pulmonary bypass procedure, endotoxemia, ischemia-reperfusion injury, cerebrovascular ischemia (stroke), renal reperfusion injury, embolism (e.g., pulmonary, renal, hepatic, gastro-intestinal, or peripheral limb embolism), or myocardial ischemia.

In certain embodiments, the disease is a viral infection. In certain embodiments, the disease is an infection caused by DNA virus. In certain embodiments, the disease is an infection caused by a dsDNA virus. In certain embodiments, the disease is an infection caused by an ssDNA virus. In certain embodiments, the disease is an infection caused by an RNA virus. In certain embodiments, the disease is an infection caused by a dsRNA virus. In certain embodiments, the disease is an infection caused by a (+)ssRNA virus. In certain embodiments, the disease is an infection caused by a (−)ssRNA virus. In certain embodiments, the disease is an infection caused by a reverse transcribing (RT) virus. In certain embodiments, the disease is an infection caused by an ssRNA-RT virus. In certain embodiments, the disease is an infection caused by a dsDNA-RT virus. In certain embodiments, the disease is an infection caused by human immunodeficiency virus (HIV). In certain embodiments, the disease is an infection caused by acquired immunodeficiency syndrome (AIDS). In certain embodiments, the disease is an infection caused by human papillomavirus (HPV). In certain embodiments, the disease is an infection caused by hepatitis C virus (HCV). In certain embodiments, the disease is an infection caused by a herpes virus (e.g., herpes simplex virus (HSV)). In certain embodiments, the disease is an infection caused by Ebola virus. In certain embodiments, the disease is an infection caused by severe acute respiratory syndrome (SARS). In certain embodiments, the disease is an infection caused by influenza virus. In certain embodiments, the disease is an infection caused by an influenza virus. In certain embodiments, the disease is an infection caused by an influenza A virus. In certain embodiments, the disease is human flu (e.g., human flu caused by H1N1, H2N2, H3N2, H5N1, H7N7, H1N2, H9N2, H7N2, H7N3, or H10N7 virus). In certain embodiments, the disease is bird flu (e.g., bird flu caused by H5N1 or H7N9 virus). In certain embodiments, the disease is swine influenza (e.g., swine influenza caused by H1N1, H1N2, H2N1, H3N1, H3N2, H2N3, or influenza C virus). In certain embodiments, the disease is equine influenza (e.g., equine influenza caused by H7N7 or H3N8 virus). In certain embodiments, the disease is canine influenza (e.g., canine influenza caused by H3N8 virus). In certain embodiments, the disease is an infection caused by an influenza B virus. In certain embodiments, the disease is an infection caused by an influenza C virus. In certain embodiments, the disease is Dengue fever, Dengue hemorrhagic fever (DHF), Dengue shock syndrome (DSS), hepatitis A, hepatitis B, hepatitis D, hepatitis E, hepatitis F, infection caused by Coxsackie A virus, infection caused by Coxsackie B virus, fulminant viral hepatitis, viral myocarditis, infection caused by parainfluenza virus, infection caused by an RS virus (RSV) (e.g., RSV bronchiolitis, RSV pneumonia, especially an infant and childhood infection caused by RSV and RSV pneumonia in the patients with cardiopulmonary disorders), infection caused by measles virus, infection caused by vesicular stomatitis virus, infection caused by rabies virus, Japanese encephalitis, infection caused by Junin virus, infection caused by human cytomegalovirus, infection caused by varicellovirus, infection caused by cytomegalovirus, infection caused by muromegalovirus, infection caused by proboscivirus, infection caused by roseolovirus, infection caused by lymphocryptovirus, infection caused by macavirus, infection caused by percavirus, infection caused by rhadinovirus, infection caused by poliovirus, infection caused by Marburg virus, infection caused by Lassa fever virus, Venezuelan equine encephalitis, infection caused by Rift Valley Fever virus, infection caused by Korean hemorrhagic fever virus, infection caused by Crimean-Congo hemorrhagic fever virus, encephalitis, Saint Louise encephalitis, Kyasanur Forest disease, Murray Valley encephalitis, tick-borne encephalitis, West Nile encephalitis, yellow fever, infection caused by adenovirus, infection caused by poxvirus, or a viral infection in subjects with immune disorders.

In certain embodiments, the disease is a bacterial infection. In certain embodiments, the bacterium is a Gram-positive bacterium. In certain embodiments, the bacterium is a multidrug-resistant bacterium. In certain embodiments, the bacterium is a Staphylococcus species. In certain embodiments, the bacterium is a Staphylococcus aureus (S. aureus) strain (e.g., ATCC 25923). In certain embodiments, the bacterium is methicillin-resistant Staphylococcus aureus (MRSA). In certain embodiments, the bacterium is the methicillin-resistant Staphylococcus aureus clinical isolate (MRSA-2, a clinical isolate from a patient treated at Shands Hospital; obtained from the Emerging Pathogens Institute at the University of Florida), such as the methicillin-resistant Staphylococcus aureus clinical isolate reported in Abouelhassan et al., Bioorg. Med. Chem. Lett., 2014, 24, 5076. In certain embodiments, the bacterium is a Staphylococcus epidermidis (S. epidermidis) strain (e.g., ATCC 12228 or ATCC 35984). In certain embodiments, the bacterium is a Staphylococcus auricularis, Staphylococcus carnosus, Staphylococcus condimenti, Staphylococcus massiliensis, Staphylococcus piscifermentans, Staphylococcus simulans, Staphylococcus capitis, Staphylococcus caprae, Staphylococcus saccharolyticus, Staphylococcus devriesei, Staphylococcus haemolyticus, Staphylococcus hominis, Staphylococcus chromogens, Staphylococcus felis, Staphylococcus delphini, Staphylococcus hyicus, Staphylococcus intermedius, Staphylococcus lutrae, Staphylococcus microti, Staphylococcus muscae, Staphylococcus pseudintermedius, Staphylococcus rostri, Staphylococcus schleiferi, Staphylococcus lugdunensis, Staphylococcus arlettae, Staphylococcus cohnii, Staphylococcus equorum, Staphylococcus gallinarum, Staphylococcus kloosii, Staphylococcus leei, Staphylococcus nepalensis, Staphylococcus saprophyticus, Staphylococcus succinus, Staphylococcus xylosus, Staphylococcus fleurettii, Staphylococcus lentus, Staphylococcus sciuri, Staphylococcus stepanovicii, Staphylococcus vitulinus, Staphylococcus simulans, Staphylococcus pasteuri, or Staphylococcus warneri strain. In certain embodiments, the bacterium is a Streptococcus species. In certain embodiments, the bacterium is a Streptococcus agalactiae, Streptococcus anginosus, Streptococcus bovis, Streptococcus canis, Streptococcus constellatus, Streptococcus dysgalactiae, Streptococcus equinus, Streptococcus iniae, Streptococcus intermedius, Streptococcus mitis, Streptococcus mutans, Streptococcus oralis, Streptococcus parasanguinis, Streptococcus peroris, Streptococcus pneumoniae, Streptococcus pseudopneumoniae, Streptococcus pyogenes, Streptococcus ratti, Streptococcus salivarius, Streptococcus tigurinus, Streptococcus thermophilus, Streptococcus sanguinis, Streptococcus sobrinus, Streptococcus suis, Streptococcus uberis, Streptococcus vestibularis, Streptococcus viridans, or Streptococcus zooepidemicus strain. In certain embodiments, the bacterium is an Enterococcus species. In certain embodiments, the bacterium is an Enterococcus avium, Enterococcus durans, Enterococcus faecalis, Enterococcus faecium, Enterococcus gallinarum, Enterococcus hirae, or Enterococcus solitarius strain. In certain embodiments, the bacterium is an Enterococcus faecium strain (e.g., a vancomycin-resistant strain of Enterococcus faecium (VRE); ATCC 700221). In certain embodiments, the bacterium is a Listeria species. In certain embodiments, the bacterium is a Listeria fleischmannii, Listeria grayi, Listeria innocua, Listeria ivanovii, Listeria marthii, Listeria monocytogenes, Listeria rocourtiae, Listeria seeligeri, Listeria weihenstephanensis, or Listeria welshimeri strain. In certain embodiments, the bacterium is a Clostridium species. In certain embodiments, the bacterium is a Clostridium acetobutylicum, Clostridium argentinense, Clostridium aerotolerans, Clostridium baratii, Clostridium beijerinckii, Clostridium bifermentans, Clostridium botulinum, Clostridium butyricum, Clostridium cadaveris, Clostridium cellulolyticum, Clostridium chauvoei, Clostridium clostridioforme, Clostridium colicanis, Clostridium difficile, Clostridium estertheticum, Clostridium fallax, Clostridium feseri, Clostridium formicaceticum, Clostridium histolyticum, Clostridium innocuum, Clostridium kluyveri, Clostridium ljungdahlii, Clostridium lavalense, Clostridium leptum, Clostridium novyi, Clostridium oedematiens, Clostridium paraputrificum, Clostridium perfringens (Alias: Clostridium welchii), Clostridium phytofermentans, Clostridium piliforme, Clostridium ragsdalei, Clostridium ramosum, Clostridium scatologenes, Clostridium septicum, Clostridium sordellii, Clostridium sporogenes, Clostridium sticklandii, Clostridium tertium, Clostridium tetani, Clostridium thermocellum, Clostridium thermosaccharolyticum, or Clostridium tyrobutyricum strain. In certain embodiments, the bacterium is a Gram-negative bacterium. In certain embodiments, the Gram-negative bacterium is an Escherichia species. In certain embodiments, the Gram-negative bacterium is an Escherichia coli (E. coli) strain (e.g., ATCC 33475, K-12, CFT073, ATCC 43895). In certain embodiments, the Gram-negative bacterium is an Escherichia albertii strain, Escherichia blattae strain, Escherichia fergusonii strain, Escherichia hermannii strain, or Escherichia vulneris strain. In certain embodiments, the Gram-negative bacterium is a Pseudomonas species. In certain embodiments, the Gram-negative bacterium is a Pseudomonas aeruginosa strain. In certain embodiments, the Gram-negative bacterium is a Pseudomonas alcaligenes strain, Pseudomonas anguilliseptica strain, Pseudomonas argentinensis strain, Pseudomonas borbori strain, Pseudomonas citronellolis strain, Pseudomonas flavescens strain, Pseudomonas mendocina strain, Pseudomonas nitroreducens strain, Pseudomonas oleovorans strain, Pseudomonas pseudoalcaligenes strain, Pseudomonas resinovorans strain, Pseudomonas straminea strain, Pseudomonas chlororaphis strain, Pseudomonas fluorescens strain, Pseudomonas pertucinogena strain, Pseudomonas putida strain, Pseudomonas stutzeri strain, or Pseudomonas syringae strain. In certain embodiments, the Gram-negative bacterium is a Klebsiella species. In certain embodiments, the Gram-negative bacterium is a Klebsiella granulomatis strain, Klebsiella oxytoca strain, Klebsiella pneumoniae strain, Klebsiella terrigena strain, or Klebsiella planticola strain. In certain embodiments, the Gram-negative bacterium is a Salmonella species. In certain embodiments, the Gram-negative bacterium is a Salmonella bongori strain or Salmonella enterica strain, e.g., Salmonella typhi. In certain embodiments, the Gram-negative bacterium is an Acinetobacter species. In certain embodiments, the Gram-negative bacterium is an Acinetobacter baumannii strain. In certain embodiments, the Gram-negative bacterium is an Acinetobacter baylyi strain, Acinetobacter bouvetii strain, Acinetobacter calcoaceticus strain, Acinetobacter gerneri strain, Acinetobacter grimontii strain, Acinetobacter haemolyticus strain, Acinetobacter johnsonii strain, Acinetobacter junii strain, Acinetobacter lwoffii strain, Acinetobacter parvus strain, Acinetobacter pittii strain, Acinetobacter radioresistens strain, Acinetobacter schindleri strain, Acinetobacter tandoii strain, Acinetobacter tjernbergiae strain, Acinetobacter towneri strain, Acinetobacter ursingii strain, or Acinetobacter gyllenbergii strain. In certain embodiments, the microorganism is a mycobacterium. In certain embodiments, the microorganism is a strain of Mycobacterium tuberculosis. In certain embodiments, the microorganism is a strain of: Mycobacterium bovis, Mycobacterium bovis BCG, Mycobacterium africanum, Mycobacterium canetti, Mycobacterium caprae, Mycobacterium microti, Mycobacterium Pinnipedii, Mycobacterium avium, Mycobacterium avium paratuberculosis, Mycobacterium avium silvaticum, Mycobacterium avium hominissuis, Mycobacterium colombiense, Mycobacterium indicus pranii, Mycobacterium gastri, Mycobacterium kansasii, Mycobacterium hiberniae, Mycobacterium nonchromogenicum, Mycobacterium terrae, Mycobacterium triviale, Mycobacterium ulcerans, Mycobacterium pseudoshottsii, Mycobacterium shottsii, Mycobacterium triplex, Mycobacterium genavense, Mycobacterium florentinum, Mycobacterium lentiflavum, Mycobacterium palustre, Mycobacterium kubicae, Mycobacterium parascrofulaceum, Mycobacterium heidelbergense, Mycobacterium interjectum, Mycobacterium simiae, Mycobacterium bohemicum, Mycobacterium botniense, Mycobacterium branderi, Mycobacterium celatum, Mycobacterium chimaera, Mycobacterium conspicuum, Mycobacterium cookii, Mycobacterium doricum, Mycobacterium farcinogenes, Mycobacterium haemophilum, Mycobacterium heckeshornense, Mycobacterium intracellulare, Mycobacterium lacus, Mycobacterium leprae, Mycobacterium lepraemurium, Mycobacterium lepromatosis, Mycobacterium malmoense, Mycobacterium marinum, Mycobacterium monacense, Mycobacterium montefiorense, Mycobacterium murale, Mycobacterium nebraskense, Mycobacterium saskatchewanense, Mycobacterium scrofulaceum, Mycobacterium shimoidei, Mycobacterium szulgai, Mycobacterium tusciae, Mycobacterium xenopi, Mycobacterium yongonense, Mycobacterium intermedium, Mycobacterium abscessus, Mycobacterium chelonae, Mycobacterium bolletii, Mycobacterium fortuitum, Mycobacterium fortuitum subsp. acetamidolyticum, Mycobacterium boenickei, Mycobacterium peregrinum, Mycobacterium porcinum, Mycobacterium senegalense, Mycobacterium septicum, Mycobacterium neworleansense, Mycobacterium houstonense, Mycobacterium mucogenicum, Mycobacterium mageritense, Mycobacterium brisbanense, Mycobacterium cosmeticum, Mycobacterium parafortuitum, Mycobacterium austroafricanum, Mycobacterium diernhoferi, Mycobacterium hodleri, Mycobacterium neoaurum, Mycobacterium frederiksbergense, Mycobacterium aurum, Mycobacterium vaccae, Mycobacterium chitae, Mycobacterium fallax, Mycobacterium confluentis, Mycobacterium flavescens, Mycobacterium madagascariense, Mycobacterium phlei, Mycobacterium smegmatis Mycobacterium goodii, Mycobacterium wolinskyi, Mycobacterium thermoresistibile, Mycobacterium gadium, Mycobacterium komossense, Mycobacterium obuense, Mycobacterium sphagni, Mycobacterium agri, Mycobacterium aichiense, Mycobacterium alvei, Mycobacterium arupense, Mycobacterium brumae, Mycobacterium canariasense, Mycobacterium chubuense, Mycobacterium conceptionense, Mycobacterium duvalii, Mycobacterium elephantis, Mycobacterium gilvum, Mycobacterium hassiacum, Mycobacterium holsaticum, Mycobacterium immunogenum, Mycobacterium massiliense, Mycobacterium moriokaense, Mycobacterium psychrotolerans, Mycobacterium pyrenivorans, Mycobacterium vanbaalenii, Mycobacterium pulveris, Mycobacterium arosiense, Mycobacterium aubagnense, Mycobacterium caprae, Mycobacterium chlorophenolicum, Mycobacterium fluoroanthenivorans, Mycobacterium kumamotonense, Mycobacterium novocastrense, Mycobacterium parmense, Mycobacterium phocaicum, Mycobacterium poriferae, Mycobacterium rhodesiae, Mycobacterium seoulense, or Mycobacterium tokaiense.

In certain embodiments, the disease is a fungal infection. In certain embodiments, the disease is a parasitic infection. In certain embodiments, the disease is a prion infection.

In certain embodiments, the disease is a fibrotic condition. In certain embodiments, the disease is selected from the group consisting of renal fibrosis, postoperative stricture, keloid formation, hepatic cirrhosis, biliary cirrhosis, and cardiac fibrosis. In certain embodiments, the disease is scleroderma. In certain embodiments, the disease is idiopathic pulmonary fibrosis.

In certain embodiments, the method further comprises administering to the subject in need thereof an additional therapy. In certain embodiments, the additional therapy is an additional pharmaceutical agent. In certain embodiments, the additional therapy is a cytotoxic chemotherapy. In certain embodiments, the additional therapy is an immunotherapy. In certain embodiments, the additional therapy is radiation therapy. The pharmaceutical compositions of the present disclosure and the additional therapy may show synergy in the methods and uses of the present disclosure.

The representative examples, which follow, are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that, unless otherwise indicated, the entire contents of each of the references cited herein are incorporated herein by reference to help illustrate the state of the art. The following examples contain important additional information, exemplification and guidance, which can be adapted to the practice of this invention in its various embodiments and the equivalents thereof.

These and other aspects of the present invention will be further appreciated upon consideration of the following Examples, which are intended to illustrate certain particular embodiments of the invention but are not intended to limit its scope, as defined by the claims.

### Examples

In order that the disclosure described herein may be more fully understood, the following examples are set forth. The synthetic and biological examples described in this application are offered to illustrate the compounds, compositions, and methods provided herein and are not to be construed in any way as limiting their scope.

Exemplary embodiments appear in Tables 1A, 2A, and 3 below with the corresponding physical data of the embodiments appearing in Tables 1B, 2B, and 4, respectively. Additional embodiments appear in Table 5, 6, and 7.

TABLE 1A
Exemplary Compositions

| | Carbopol | PEG- | HPC- | Methyl | | | | | | Flavor | | PH |
|---|---|---|---|---|---|---|---|---|---|---|---|---|
| # | 971P NF | 400 | M | Cellulose | Glycerol | EtOH | H₂O | Cavitron | Cavasol | Agent | Colorant | adjuster |
| 1 | 0.50 | 10.00 | 0.00 | 0.00 | 3.00 | 38.50 | 48.00 | 0.00 | 0.00 | 0.00 | 0.00 | — |
| 2 | 0.50 | 8.50 | 0.00 | 0.00 | 3.00 | 40.00 | 48.00 | 0.00 | 0.00 | 0.00 | 0.00 | — |
| 3 | 0.50 | 10.00 | 0.00 | 0.00 | 3.00 | 31.50 | 55.00 | 0.00 | 0.00 | 0.00 | 0.00 | — |
| 4 | 0.50 | 4.50 | 0.00 | 0.00 | 0.00 | 40.00 | 55.00 | 0.00 | 0.00 | 0.00 | 0.00 | — |
| 5 | 0.50 | 1.50 | 0.00 | 0.00 | 3.00 | 40.00 | 55.00 | 0.00 | 0.00 | 0.00 | 0.00 | — |
| 6 | 0.50 | 9.99 | 0.00 | 0.00 | 0.00 | 40.00 | 49.51 | 0.00 | 0.00 | 0.00 | 0.00 | — |
| 7 | 3.00 | 20.00 | 0.00 | 0.00 | 5.00 | 40.00 | 32.00 | 0.00 | 0.00 | 0.00 | 0.00 | Q.S. |
| 8 | 2.00 | 20.00 | 0.00 | 0.00 | 0.00 | 21.00 | 57.00 | 0.00 | 0.00 | 0.00 | 0.00 | Q.S. |
| 9 | 2.00 | 5.00 | 0.00 | 3.00 | 0.00 | 33.00 | 57.00 | 0.00 | 0.00 | 0.00 | 0.00 | Q.S. |
| 10 | 0.30 | 7.92 | 0.00 | 1.78 | 0.00 | 33.00 | 57.00 | 0.00 | 0.00 | 0.00 | 0.00 | Q.S. |
| 11 | 0.46 | 6.54 | 0.00 | 3.00 | 0.00 | 35.17 | 54.83 | 0.00 | 0.00 | 0.00 | 0.00 | Q.S. |
| 12 | 0.40 | 9.60 | 0.00 | 0.00 | 1.07 | 31.39 | 57.00 | 0.00 | 0.00 | 0.00 | 0.00 | Q.S. |
| 13 | 0.50 | 9.50 | 0.00 | 0.00 | 0.93 | 34.30 | 54.19 | 0.00 | 0.00 | 0.00 | 0.00 | Q.S. |
| 14 | 0.30 | 7.90 | 0.00 | 1.78 | 0.00 | 32.98 | 57.04 | 0.00 | 0.00 | 0.00 | 0.00 | — |
| 15 | 0.28 | 7.49 | 0.00 | 1.68 | 0.00 | 31.23 | 53.93 | 5.39 | 0.00 | 0.00 | 0.00 | — |
| 16 | 0.22 | 5.66 | 0.00 | 0.00 | 0.00 | 23.90 | 41.16 | 0.00 | 29.20 | 0.00 | 0.00 | — |
| 17 | 0.30 | 7.91 | 0.00 | 1.76 | 0.00 | 32.90 | 56.92 | 0.00 | 0.00 | 0.00 | 0.00 | — |
| 18 | 0.30 | 7.88 | 0.00 | 1.76 | 0.00 | 32.91 | 56.74 | 0.00 | 0.00 | 0.00 | 0.00 | — |
| 19 | 0.29 | 7.59 | 0.00 | 1.68 | 0.00 | 31.74 | 54.54 | 0.00 | 0.00 | 0.00 | 0.00 | — |
| 20 | 0.30 | 7.92 | 1.78 | 0.00 | 0.00 | 33.03 | 56.97 | 0.00 | 0.00 | 0.00 | 0.00 | — |
| 21 | 0.30 | 7.95 | 1.78 | 0.00 | 0.00 | 32.99 | 56.98 | 0.00 | 0.00 | 0.00 | 0.00 | — |
| 22 | 0.30 | 7.87 | 1.78 | 0.00 | 0.00 | 33.00 | 56.93 | 0.00 | 0.00 | 0.00 | 0.12 | — |
| 23 | 0.35 | 7.89 | 0.00 | 0.00 | 0.00 | 33.87 | 57.89 | 0.00 | 0.00 | 0.00 | 0.00 | — |
| 24 | 0.35 | 7.89 | 0.00 | 0.00 | 0.00 | 33.86 | 57.87 | 0.00 | 0.00 | 1.01 | 0.03 | — |
| 25 | 0.30 | 7.89 | 1.77 | 0.00 | 0.00 | 32.93 | 57.10 | 0.00 | 0.00 | 0.00 | 0.00 | — |
| 26 | 0.30 | 7.81 | 1.75 | 0.00 | 0.00 | 32.59 | 56.51 | 0.00 | 0.00 | 1.01 | 0.03 | — |
| 27 | 0.40 | 7.88 | 1.77 | 0.00 | 0.00 | 32.89 | 57.03 | 0.00 | 0.00 | 1.01 | 0.03 | — |
| 28 | 0.90 | 1.84 | 0.40 | 0.00 | 0.00 | 38.98 | 57.88 | 0.00 | 0.00 | 0.00 | 0.00 | — |
| 29 | 0.89 | 1.78 | 0.40 | 0.00 | 0.00 | 38.60 | 57.31 | 0.00 | 0.00 | 1.00 | 0.03 | — |
| 30 | 0.51 | 6.08 | 0.00 | 0.00 | 0.00 | 23.16 | 40.17 | 0.00 | 30.08 | 0.00 | 0.00 | — |
| 31 | 0.30 | 6.35 | 0.00 | 0.00 | 0.00 | 0.00 | 62.16 | 0.00 | 29.56 | 0.00 | 0.00 | — |
| 32 | 0.50 | 6.09 | 0.00 | 0.00 | 0.00 | 0.00 | 62.22 | 0.00 | 29.56 | 0.00 | 0.00 | — |
| 33 | 0.49 | 7.70 | 0.00 | 0.00 | 0.00 | 0.00 | 62.27 | 0.00 | 27.96 | 0.00 | 0.00 | — |
| 34 | 0.30 | 7.73 | 0.00 | 0.00 | 0.00 | 0.00 | 62.53 | 0.00 | 27.95 | 0.00 | 0.00 | — |
| 35 | 0.30 | 20.00 | 0.00 | 0.00 | 0.00 | 24.68 | 55.02 | 0.00 | 0.00 | 0.00 | 0.00 | — |
| 36 | 0.30 | 39.98 | 0.00 | 0.00 | 0.00 | 19.69 | 40.04 | 0.00 | 0.00 | 0.00 | 0.00 | — |
| 37 | 0.30 | 40.02 | 0.00 | 0.00 | 0.00 | 19.65 | 40.02 | 0.00 | 0.00 | 0.00 | 0.00 | — |
| 38 | 0.30 | 76.47 | 0.00 | 0.00 | 0.00 | 12.71 | 10.53 | 0.00 | 0.00 | 0.00 | 0.00 | — |

Q.S. = quantum satis,
pH was adjusted using NaOH or HCl

TABLE 1B
Properties of Exemplary Compositions
Appearing in Table 1A

| | Mean Viscosity | | | |
|---|---|---|---|---|
| # | (mPas) | Sprayability | Mucoadhesion | pH |
| 1 | 646.67 | Yes | Yes | 4.97 |
| 2 | 650.00 | Yes | Yes | 4.86 |
| 3 | 886.67 | Yes | Yes | 4.68 |
| 4 | 540.00 | Yes | Yes | 4.49 |
| 5 | 496.67 | Yes | Yes | 4.36 |
| 6 | 660.00 | Yes | Yes | 4.82 |
| 7 | 16633.33 | No | Yes | 3.92 |
| 8 | 6533.33 | No | Yes | 4.27 |
| 9 | 73383.33 | No | Yes | 4.11 |
| 10 | 553.67 | Yes | Yes | 4.91 |
| 11 | 2286.67 | No | Yes | 4.65 |
| 12 | 15060.00 | Yes | Yes | 6.29 |
| 13 | 2620.00 | Yes | Yes | 6.18 |
| 14 | 488.33 | Yes | Yes | 4.85 |
| 15 | 502.67 | Yes | Yes | 4.85 |
| 16 | 128.67 | Yes | Yes | 4.91 |
| 17 | 115.67 | Yes | Yes | 4.75 |
| 18 | 80.00 | Yes | Yes | 4.49 |
| 19 | 65.97 | Yes | Yes | 4.04 |
| 20 | NT | Yes | NT | NT |
| 21 | NT | Yes | NT | NT |
| 22 | NT | Yes | NT | NT |
| 23 | NT | Yes | NT | NT |
| 24 | NT | Yes | NT | 4.48 |
| 25 | NT | Yes | NT | NT |
| 26 | 212.33 | Yes | Yes | 4.77 |
| 27 | 351.33 | Yes | Yes | 4.69 |
| 28 | NT | Yes | Yes | 6.79 |
| 29 | NT | Yes | Yes | NT |
| 30 | 1175.33 | NT | Yes | 4.53 |
| 31 | 3011.33 | Yes | Yes | 4.51 |
| 32 | 636.67 | NT | Yes | 4.32 |
| 33 | NT | NT | Yes | NT |
| 34 | NT | Yes | Yes | NT |
| 35 | 1147.00 | Yes | Yes | 5.22 |
| 36 | 1248.67 | Yes | Yes | 6.19 |
| 37 | NT | NT | NT | NT |
| 38 | NT | Yes | Yes | 7.31 |

NT = Not tested

TABLE 2A
Exemplary Compositions

| | | PVP- | | | | | | | | | | |
|---|---|---|---|---|---|---|---|---|---|---|---|---|
| | PEG- | VA- | HPC- | HPC- | HPC- | | | | Citric | | | pH |
| # | 400 | 64 | M | G | J | Glycerol | EtOH | H₂O | Acid | Sucrose | Peppermint | adjuster |
| 40 | 19.96 | 9.96 | 0 | 0 | 0 | 0.00 | 19.96 | 50.12 | 0.00 | 0.00 | 0.00 | — |
| 41 | 39.99 | 9.99 | 0 | 0 | 0 | 0.00 | 20.02 | 30.00 | 0.00 | 0.00 | 0.00 | — |
| 42 | 74.99 | 10.02 | 0 | 0 | 0 | 0.00 | 4.50 | 10.50 | 0.00 | 0.00 | 0.00 | — |
| 43 | 76.56 | 8.35 | 0 | 0 | 0 | 0.00 | 4.86 | 10.22 | 0.00 | 0.00 | 0.00 | — |
| 44 | 66.28 | 7.97 | 0 | 0 | 0 | 0.00 | 15.45 | 10.29 | 0.00 | 0.00 | 0.00 | — |
| 45 | 5.09 | 0.72 | 0.60 | 0.90 | 0 | 0.92 | 38.98 | 51.92 | 0.87 | 0.00 | 0.00 | — |
| 46 | 5.34 | 1.51 | 0.60 | 0.90 | 0 | 0.00 | 39.01 | 50.95 | 1.70 | 0.00 | 0.00 | — |
| 47 | 19.96 | 9.96 | 0 | 0 | 0 | 0.00 | 19.96 | 50.12 | 0.00 | 0.00 | 0.00 | — |
| 48 | 39.99 | 9.99 | 0 | 0 | 0 | 0.00 | 20.02 | 30.00 | 0.00 | 0.00 | 0.00 | — |
| 49 | 74.99 | 10.02 | 0 | 0 | 0 | 0.00 | 4.50 | 10.50 | 0.00 | 0.00 | 0.00 | — |
| 50 | 76.56 | 8.35 | 0 | 0 | 0 | 0.00 | 4.86 | 10.22 | 0.00 | 0.00 | 0.00 | — |
| 51 | 66.28 | 7.97 | 0 | 0 | 0 | 0.00 | 15.45 | 10.29 | 0.00 | 0.00 | 0.00 | — |
| 52 | 77.04 | 8.46 | 0 | 0 | 0 | 0.00 | 4.03 | 10.47 | 0.00 | 0.00 | 0.00 | — |
| 53 | 66.39 | 8.07 | 0 | 0 | 0 | 0.00 | 15.33 | 10.21 | 0.00 | 0.00 | 0.00 | — |
| 54 | 66.39 | 7.92 | 0 | 0 | 0 | 0.00 | 15.31 | 10.25 | 0.12 | 0.00 | 0.00 | — |
| 55 | 66.33 | 8.06 | 0 | 0 | 0 | 0.00 | 15.26 | 10.23 | 0.12 | 0.00 | 0.00 | — |
| 56 | 66.35 | 8.06 | 0 | 0 | 0 | 0.00 | 15.35 | 10.24 | 0.00 | 0.00 | 0.00 | — |
| 57 | 53.83 | 10.04 | 0 | 0 | 0 | 3.21 | 15.16 | 17.67 | 0.00 | 0.10 | 0.00 | — |
| 58 | 66.09 | 8.04 | 0 | 0 | 0 | 0.00 | 15.17 | 10.15 | 0.12 | 0.00 | 0.00 | — |
| 59 | 66.37 | 8.08 | 0 | 0.45 | 0 | 0.00 | 15.23 | 10.20 | 0.12 | 0.00 | 0.00 | — |
| 60 | 73.95 | 9.88 | 0 | 0 | 0 | 0.00 | 4.44 | 10.35 | 0.00 | 0.00 | 0.00 | — |
| 61 | 73.95 | 9.89 | 0 | 0 | 0 | 0.00 | 4.45 | 10.34 | 0.00 | 0.00 | 0.00 | — |
| 62 | 73.98 | 9.89 | 0 | 0 | 0 | 0.00 | 4.42 | 10.33 | 0.00 | 0.00 | 0.00 | Q.S. |
| 63 | 73.98 | 9.89 | 0 | 0 | 0 | 0.00 | 4.42 | 10.33 | 0.00 | 0.00 | 0.00 | Q.S. |
| 64 | 74.00 | 9.90 | 0 | 0 | 0 | 0.00 | 4.40 | 10.30 | 0.00 | 0.00 | 0.00 | Q.S. |
| 65 | 73.96 | 9.94 | 0 | 0 | 0 | 0.00 | 4.37 | 10.34 | 0.00 | 0.00 | 0.00 | Q.S. |
| 66 | 75.09 | 10.01 | 0 | 0 | 0 | 0.00 | 4.50 | 7.90 | 0.00 | 0.00 | 0.00 | — |
| 67 | 63.00 | 9.99 | 0 | 0 | 0 | 0.00 | 14.98 | 10.03 | 0.00 | 0.00 | 0.00 | — |
| 68 | 74.98 | 8.09 | 0 | 0 | 0 | 0.00 | 4.53 | 9.99 | 0.00 | 0.00 | 0.00 | — |
| 69 | 64.95 | 7.89 | 0 | 0 | 0 | 0.00 | 15.02 | 10.03 | 0.00 | 0.00 | 0.00 | — |
| 70 | 64.96 | 7.77 | 0 | 0 | 0 | 0.00 | 14.99 | 10.06 | 0.00 | 0.00 | 0.00 | — |
| 71 | 64.87 | 7.89 | 0 | 0 | 0 | 0.00 | 15.17 | 9.98 | 0.00 | 0.00 | 0.00 | — |
| 72 | 64.84 | 7.90 | 0 | 0 | 0 | 0.00 | 14.96 | 10.09 | 0.00 | 0.00 | 0.00 | — |
| 73 | 64.91 | 7.90 | 0 | 0 | 0 | 0.00 | 14.92 | 10.05 | 0.00 | 0.00 | 0.00 | — |
| 74 | 65.02 | 7.90 | 0 | 0 | 0 | 0.00 | 14.87 | 9.99 | 0.00 | 0.00 | 0.00 | — |
| 75 | 64.92 | 7.87 | 0 | 0 | 0 | 0.00 | 14.94 | 10.06 | 0.00 | 0.00 | 0.00 | — |
| 76 | 64.95 | 7.89 | 0 | 0 | 0 | 0.00 | 14.87 | 10.08 | 0.00 | 0.00 | 0.00 | — |
| 77 | 65.00 | 7.88 | 0 | 0 | 0 | 0.00 | 14.75 | 10.17 | 0.00 | 0.00 | 0.00 | — |
| 78 | 64.99 | 7.90 | 0 | 0 | 0 | 0.00 | 14.89 | 10.00 | 0.12 | 0.00 | 0.00 | — |
| 79 | 64.99 | 7.90 | 0 | 0 | 0 | 0.00 | 14.89 | 10.00 | 0.12 | 0.00 | 0.00 | — |
| 80 | 64.99 | 7.90 | 0 | 0 | 0 | 0.00 | 14.89 | 10.00 | 0.12 | 0.00 | 0.00 | — |
| 81 | 64.96 | 7.89 | 0 | 0 | 0 | 0.00 | 14.93 | 10.00 | 0.12 | 0.00 | 0.00 | — |
| 82 | 64.86 | 7.87 | 0 | 0 | 0.20 | 0.00 | 14.90 | 9.98 | 0.12 | 0.00 | 0.00 | — |
| 83 | 66.38 | 8.07 | 0 | 0 | 0 | 0.00 | 15.21 | 10.23 | 0.12 | 0.00 | 0.00 | — |
| 84 | 66.36 | 8.08 | 0 | 0 | 0.20 | 0.00 | 15.20 | 10.24 | 0.12 | 0.00 | 0.00 | — |
| 85 | 66.34 | 8.07 | 0 | 0 | 0 | 0.00 | 15.20 | 10.27 | 0.12 | 0.00 | 0.00 | — |
| 86 | 64.69 | 8.06 | 0 | 0 | 0 | 1.54 | 15.36 | 10.23 | 0.12 | 0.00 | 0.00 | — |
| 87 | 55.17 | 8.06 | 0 | 0 | 0 | 3.07 | 10.20 | 23.32 | 0.12 | 0.05 | 0.00 | — |
| 88 | 66.23 | 10.19 | 0 | 0 | 0 | 3.07 | 10.19 | 10.19 | 0.12 | 0.00 | 0.00 | — |
| 89 | 66.36 | 10.20 | 0 | 0 | 0 | 1.53 | 10.23 | 11.50 | 0.12 | 0.05 | 0.00 | — |
| 90 | 59.84 | 9.91 | 0 | 0 | 0 | 2.25 | 12.15 | 15.73 | 0.12 | 0.00 | 0.00 | — |
| 91 | 60.03 | 9.87 | 0 | 0 | 0 | 2.18 | 12.18 | 15.59 | 0.12 | 0.03 | 0.00 | — |
| 92 | 59.91 | 10.19 | 0 | 0 | 0 | 0.00 | 14.06 | 15.69 | 0.12 | 0.03 | 0.00 | — |
| 93 | 59.39 | 10.18 | 0 | 0 | 0 | 3.10 | 12.18 | 14.97 | 0.12 | 0.05 | 0.00 | — |
| 94 | 61.10 | 10.18 | 0 | 0 | 0 | 3.09 | 12.68 | 12.78 | 0.12 | 0.05 | 0.00 | — |
| 95 | 62.53 | 10.19 | 0 | 0 | 0 | 3.05 | 13.58 | 10.49 | 0.12 | 0.05 | 0.00 | — |
| 96 | 63.60 | 10.15 | 0 | 0 | 0 | 3.08 | 12.64 | 10.35 | 0.12 | 0.05 | 0.00 | — |
| 97 | 63.81 | 10.06 | 0 | 0 | 0 | 1.90 | 12.92 | 11.14 | 0.12 | 0.05 | 0.00 | — |
| 98 | 65.00 | 8.00 | 0 | 0 | 0 | 0.00 | 15.00 | 10.00 | 0.00 | 0.00 | 0.00 | — |
| 99 | 65.00 | 8.00 | 0 | 0 | 0 | 0.00 | 15.00 | 10.00 | 0.00 | 0.00 | 0.00 | Q.S. |
| 100 | 65.00 | 8.00 | 0 | 0 | 0 | 0.00 | 15.00 | 10.00 | 0.00 | 0.00 | 0.00 | — |
| 101 | 63.20 | 10.00 | 0 | 0 | 0 | 1.59 | 13.91 | 10.14 | 0.12 | 0.03 | 0.05 | — |
| 102 | 61.36 | 9.94 | 0 | 0 | 0 | 1.92 | 14.38 | 10.54 | 0.12 | 0.03 | 0.86 | — |
| 103 | 61.68 | 9.96 | 0 | 0 | 0 | 1.53 | 14.08 | 10.09 | 0.12 | 0.03 | 1.25 | — |
| 104 | 63.01 | 9.95 | 0 | 0 | 0 | 1.53 | 14.26 | 10.08 | 0.12 | 0.00 | 0.51 | — |
| 105 | 61.35 | 9.88 | 0 | 0 | 0 | 1.45 | 13.84 | 12.32 | 0.12 | 0.03 | 0.50 | — |
| 106 | 61.58 | 9.93 | 0 | 0 | 0 | 1.52 | 14.90 | 10.20 | 0.12 | 0.00 | 1.25 | — |
| 107 | 60.82 | 9.98 | 0 | 0 | 0 | 1.60 | 15.04 | 10.65 | 0.12 | 0.03 | 0.50 | — |
| 108 | 60.98 | 10.01 | 0 | 0 | 0 | 2.80 | 15.01 | 10.01 | 0.12 | 0.00 | 0.52 | — |
| 109 | 60.64 | 9.94 | 0 | 0 | 0 | 1.59 | 14.18 | 11.00 | 0.12 | 0.00 | 1.25 | — |
| 110 | 60.83 | 9.98 | 0 | 0 | 0 | 2.93 | 14.11 | 10.18 | 0.12 | 0.03 | 1.28 | — |
| 111 | 60.77 | 9.91 | 0 | 0 | 0 | 2.91 | 14.53 | 9.97 | 0.12 | 0.00 | 0.51 | — |
| 112 | 60.82 | 9.97 | 0 | 0 | 0 | 1.53 | 14.02 | 12.50 | 0.12 | 0.00 | 0.52 | — |
| 113 | 61.63 | 9.96 | 0 | 0 | 0 | 2.92 | 12.55 | 10.29 | 0.12 | 0.00 | 1.26 | — |
| 114 | 62.02 | 9.79 | 0 | 0 | 0 | 0.00 | 15.67 | 9.92 | 0.12 | 0.00 | 1.23 | — |
| 115 | 61.12 | 9.87 | 0 | 0 | 0 | 1.58 | 14.80 | 10.00 | 0.12 | 0.01 | 1.25 | — |
| 116 | 66.39 | 8.03 | 0 | 0 | 0 | 0.00 | 15.43 | 10.15 | 0.00 | 0.00 | 0.00 | — |
| 117 | 66.44 | 8.07 | 0 | 0 | 0 | 0.00 | 15.18 | 10.19 | 0.12 | 0.00 | 0.00 | — |
| 118 | 65.00 | 7.88 | 0 | 0 | 0 | 0.00 | 15.05 | 9.96 | 0.00 | 0.00 | 0.00 | — |
| 119 | 65.04 | 7.90 | 0 | 0 | 0 | 0.00 | 14.86 | 9.97 | 0.12 | 0.00 | 0.00 | — |

Q.S. = quantum satis,
pH was adjusted using NaOH or HCl

TABLE 2B
Properties of Exemplary Compositions
Appearing in Table 2A

| | Mean Viscosity | | | |
|---|---|---|---|---|
| # | (mPas) | Sprayability | Mucoadhesion | pH |
| 40 | 211.00 | Yes | Yes | 5.96 |
| 41 | NT | NT | NT | NT |
| 42 | NT | Yes | Yes | 8.16 |
| 43 | NT | NT | Yes | 8.32 |
| 44 | NT | NT | Yes | 8.19 |
| 45 | 1198.00 | No | Yes | 2.94 |
| 46 | 1328.00 | No | Yes | 2.74 |
| 47 | 211.00 | Yes | Yes | 5.96 |
| 48 | 440.00 | Yes | Yes | 7.06 |
| 49 | NT | Yes | Yes | 8.16 |
| 50 | NT | NT | Yes | 8.32 |
| 51 | NT | NT | Yes | 8.19 |
| 52 | NT | Yes | Yes | NT |
| 53 | NT | Yes | Yes | NT |
| 54 | NT | Yes | Yes | NT |
| 55 | NT | Yes | Yes | NT |
| 56 | NT | Yes | Yes | NT |
| 57 | NT | Yes | Yes | NT |
| 58 | NT | Yes | Yes | NT |
| 59 | NT | Yes | Yes | NT |
| 60 | NT | Yes | Yes | 8.30 |
| 61 | 307.33 | Yes | Yes | 8.40 |
| 62 | 306.67 | Yes | Yes | 2.20 |
| 63 | 291.67 | Yes | Yes | 2.49 |
| 64 | NT | Yes | Yes | NT |
| 65 | NT | Yes | Yes | NT |
| 66 | NT | Yes | Yes | NT |
| 67 | 106.00 | Yes | Yes | 7.95 |
| 68 | 115.57 | Yes | Yes | 8.26 |
| 69 | 247.33 | Yes | Yes | 8.09 |
| 70 | NT | Yes | Yes | NT |
| 71 | NT | Yes | Yes | NT |
| 72 | NT | Yes | NT | 6.62 |
| 73 | NT | Yes | NT | 5.74 |
| 74 | NT | Yes | NT | 5.65 |
| 75 | NT | Yes | NT | 5.64 |
| 76 | NT | Yes | NT | 5.68 |
| 77 | NT | Yes | NT | 5.71 |
| 78 | 116.23 | Yes | Yes | 6.60 |
| 79 | 116.23 | Yes | Yes | 6.60 |
| 80 | 116.23 | Yes | Yes | 6.60 |
| 81 | NT | Yes | Yes | NT |
| 82 | NT | Yes | Yes | NT |
| 83 | NT | Yes | Yes | NT |
| 84 | NT | Yes | Yes | NT |
| 85 | NT | Yes | Yes | NT |
| 86 | NT | Yes | Yes | 5.44 |
| 87 | NT | Yes | Yes | 5.09 |
| 88 | NT | Yes | Yes | 5.39 |
| 89 | NT | Yes | Yes | 5.57 |
| 90 | NT | Yes | Yes | 5.37 |
| 91 | NT | Yes | Yes | NT |
| 92 | NT | Yes | Yes | NT |
| 93 | NT | Yes | Yes | NT |
| 94 | NT | Yes | Yes | NT |
| 95 | NT | Yes | Yes | NT |
| 96 | NT | Yes | Yes | NT |
| 97 | NT | Yes | Yes | NT |
| 98 | 306.00 | Yes | Yes | 8.35 |
| 99 | 306.00 | Yes | Yes | 2.78 |
| 100 | 306.00 | Yes | Yes | 8.35 |
| 101 | NT | Yes | NT | NT |
| 102 | NT | Yes | NT | NT |
| 103 | NT | Yes | NT | 5.55 |
| 104 | NT | Yes | NT | NT |
| 105 | NT | Yes | NT | NT |
| 106 | NT | Yes | NT | NT |
| 107 | NT | Yes | NT | NT |
| 108 | NT | Yes | NT | NT |
| 109 | NT | Yes | NT | NT |
| 110 | NT | Yes | NT | NT |
| 111 | NT | Yes | NT | NT |
| 112 | NT | Yes | NT | NT |
| 113 | NT | Yes | NT | 5.53 |
| 114 | NT | Yes | NT | 5.55 |
| 115 | NT | Yes | NT | 5.65 |
| 116 | NT | Yes | NT | NT |
| 117 | NT | Yes | NT | NT |
| 118 | NT | Yes | NT | NT |
| 119 | NT | Yes | NT | NT |

NT = Not tested

TABLE 3
Exemplary Compositions

| | | | | PVP- | | | | | | | | | | | |
|---|---|---|---|---|---|---|---|---|---|---|---|---|---|---|---|
| | Carbopol | PEG- | PVP- | VA- | HPC- | HPC- | Methyl | | | | Salicylic | Citric | | Flavor | |
| # | 971P NF | 400 | 30K | 64 | M | G | cellulose | Glycerol | EtOH | H₂O | Acid | Acid | Cavitron | Agent | Colorant |
| 120 | 0.00 | 0.00 | 0.00 | 2.02 | 1.22 | 0.00 | 0.00 | 2.58 | 35.97 | 57.86 | 0.52 | 0.00 | 0.00 | 0.00 | 0.00 |
| 121 | 0.00 | 0.00 | 0.00 | 0.13 | 1.41 | 0.00 | 0.00 | 1.10 | 37.97 | 58.99 | 0.50 | 0.00 | 0.00 | 0.00 | 0.00 |
| 122 | 0.00 | 0.00 | 0.00 | 0.00 | 0.00 | 2.00 | 0.00 | 0.14 | 38.33 | 59.03 | 0.50 | 0.00 | 0.00 | 0.00 | 0.00 |
| 123 | 0.00 | 0.00 | 0.00 | 1.10 | 1.81 | 0.60 | 0.00 | 1.20 | 39.02 | 55.77 | 0.50 | 0.00 | 0.00 | 0.00 | 0.00 |
| 124 | 0.00 | 0.00 | 0.00 | 1.26 | 0.48 | 1.77 | 0.00 | 2.94 | 38.65 | 53.93 | 0.99 | 0.00 | 0.00 | 0.00 | 0.00 |
| 125 | 0.00 | 0.00 | 0.00 | 1.76 | 0.97 | 2.25 | 0.00 | 0.00 | 39.15 | 54.44 | 1.49 | 0.00 | 0.00 | 0.00 | 0.00 |
| 126 | 0.00 | 0.00 | 0.00 | 1.35 | 0.00 | 1.83 | 0.00 | 3.02 | 38.75 | 54.04 | 1.07 | 0.00 | 0.00 | 0.00 | 0.00 |
| 127 | 0.00 | 0.00 | 0.00 | 1.34 | 0.00 | 1.84 | 0.00 | 3.01 | 38.76 | 54.04 | 1.09 | 0.00 | 0.00 | 0.00 | 0.00 |
| 128 | 0.00 | 0.00 | 0.00 | 1.34 | 0.00 | 1.84 | 0.00 | 3.01 | 38.76 | 54.04 | 1.09 | 0.00 | 0.00 | 0.00 | 0.00 |
| 129 | 0.00 | 0.00 | 0.00 | 1.35 | 0.00 | 1.84 | 0.00 | 3.04 | 38.78 | 54.05 | 1.09 | 0.00 | 0.00 | 0.00 | 0.00 |
| 130 | 0.00 | 0.00 | 0.00 | 1.34 | 1.83 | 0.00 | 0.00 | 3.02 | 38.74 | 53.99 | 1.06 | 0.00 | 0.00 | 0.00 | 0.00 |
| 131 | 0.00 | 0.00 | 0.00 | 1.17 | 0.79 | 1.08 | 0.00 | 2.72 | 38.48 | 54.66 | 1.11 | 0.00 | 0.00 | 0.00 | 0.00 |
| 132 | 0.00 | 0.00 | 0.00 | 1.52 | 0.63 | 0.90 | 0.00 | 2.91 | 39.55 | 53.24 | 1.25 | 0.00 | 0.00 | 0.00 | 0.00 |
| 133 | 0.00 | 0.00 | 0.00 | 1.52 | 0.63 | 0.90 | 0.00 | 2.92 | 39.54 | 53.24 | 1.25 | 0.00 | 0.00 | 0.00 | 0.00 |
| 134 | 0.00 | 0.00 | 0.00 | 1.49 | 0.41 | 1.15 | 0.00 | 2.92 | 39.29 | 53.39 | 1.41 | 0.00 | 0.00 | 0.00 | 0.00 |
| 135 | 0.00 | 0.00 | 0.00 | 1.49 | 0.41 | 1.15 | 0.00 | 2.92 | 39.29 | 53.36 | 1.42 | 0.00 | 0.00 | 0.00 | 0.00 |
| 136 | 0.00 | 0.00 | 0.00 | 1.69 | 0.00 | 1.73 | 0.00 | 2.92 | 38.31 | 53.97 | 1.42 | 0.00 | 0.00 | 0.00 | 0.00 |
| 137 | 0.00 | 0.00 | 0.00 | 1.69 | 0.00 | 1.73 | 0.00 | 2.94 | 38.32 | 54.00 | 1.42 | 0.00 | 0.00 | 0.00 | 0.00 |
| 138 | 0.00 | 0.00 | 0.00 | 1.20 | 0.64 | 0.00 | 0.00 | 2.83 | 36.49 | 54.34 | 1.36 | 0.00 | 0.00 | 0.00 | 0.00 |
| 139* | 0.00 | 0.00 | 0.00 | 1.52 | 0.63 | 0.90 | 0.00 | 2.93 | 39.56 | 53.29 | 1.25 | 0.00 | 0.00 | 0.00 | 0.00 |
| 140 | 0.00 | 0.00 | 0.00 | 0.95 | 1.11 | 0.22 | 0.00 | 2.07 | 38.08 | 56.65 | 0.96 | 0.00 | 0.00 | 0.00 | 0.00 |
| 141 | 0.00 | 0.00 | 0.00 | 1.52 | 0.63 | 0.92 | 0.00 | 2.91 | 39.56 | 53.25 | 1.25 | 0.00 | 0.00 | 0.00 | 0.00 |
| 142 | 0.00 | 0.00 | 0.00 | 1.52 | 0.63 | 0.90 | 0.00 | 2.92 | 39.54 | 53.34 | 0.00 | 1.25 | 0.00 | 0.00 | 0.00 |
| 143 | 0.00 | 0.00 | 0.00 | 1.52 | 0.63 | 0.90 | 0.00 | 3.55 | 39.55 | 53.85 | 0.00 | 0.00 | 0.00 | 0.00 | 0.00 |
| 144 | 0.00 | 8.50 | 0.00 | 0.00 | 0.60 | 0.90 | 0.00 | 0.00 | 39.03 | 50.97 | 0.00 | 0.00 | 0.00 | 0.00 | 0.00 |
| 145 | 0.00 | 0.00 | 0.00 | 0.00 | 0.60 | 0.90 | 0.00 | 1.00 | 39.00 | 56.80 | 0.00 | 1.70 | 0.00 | 0.00 | 0.00 |
| 146 | 0.00 | 0.00 | 0.00 | 0.00 | 0.60 | 0.90 | 0.00 | 0.00 | 38.99 | 59.51 | 0.00 | 0.00 | 0.00 | 0.00 | 0.00 |
| 147 | 0.00 | 5.09 | 0.00 | 0.72 | 0.60 | 0.90 | 0.00 | 0.92 | 38.98 | 51.92 | 0.00 | 0.87 | 0.00 | 0.00 | 0.00 |
| 148 | 0.00 | 5.34 | 0.00 | 1.51 | 0.60 | 0.90 | 0.00 | 0.00 | 39.01 | 50.95 | 0.00 | 1.70 | 0.00 | 0.00 | 0.00 |
| 149 | 0.00 | 0.00 | 0.00 | 1.51 | 0.59 | 0.90 | 0.00 | 1.06 | 39.00 | 56.95 | 0.00 | 0.00 | 0.00 | 0.00 | 0.00 |
| 150 | 0.00 | 0.00 | 0.00 | 0.00 | 0.60 | 0.90 | 0.00 | 0.00 | 38.98 | 57.82 | 0.00 | 1.70 | 0.00 | 0.00 | 0.00 |
| 151 | 0.00 | 0.00 | 0.00 | 1.51 | 0.60 | 0.90 | 0.00 | 0.00 | 38.99 | 58.00 | 0.00 | 0.00 | 0.00 | 0.00 | 0.00 |
| 152 | 0.00 | 1.80 | 0.00 | 0.00 | 0.60 | 0.70 | 0.00 | 0.00 | 38.96 | 57.95 | 0.00 | 0.00 | 0.00 | 0.00 | 0.00 |
| 153 | 0.00 | 1.23 | 0.00 | 0.00 | 0.50 | 0.80 | 0.00 | 0.00 | 39.04 | 57.31 | 0.00 | 1.13 | 0.00 | 0.00 | 0.00 |
| 154 | 0.00 | 0.00 | 0.00 | 0.00 | 0.60 | 0.90 | 0.00 | 0.00 | 39.00 | 57.90 | 0.00 | 1.60 | 0.00 | 0.00 | 0.00 |
| 155 | 0.00 | 1.82 | 0.00 | 0.00 | 0.40 | 0.90 | 0.00 | 0.00 | 39.01 | 57.87 | 0.00 | 0.00 | 0.00 | 0.00 | 0.00 |
| 156 | 0.00 | 1.80 | 0.00 | 0.00 | 0.60 | 0.90 | 0.00 | 0.00 | 39.03 | 55.98 | 0.00 | 1.70 | 0.00 | 0.00 | 0.00 |
| 157 | 0.00 | 0.63 | 0.00 | 0.00 | 0.40 | 0.70 | 0.00 | 0.00 | 38.99 | 57.88 | 0.00 | 1.40 | 0.00 | 0.00 | 0.00 |
| 158 | 0.00 | 1.70 | 0.00 | 0.00 | 0.38 | 0.85 | 0.00 | 0.00 | 36.86 | 54.74 | 0.00 | 0.00 | 5.47 | 0.00 | 0.00 |
| 159 | 0.00 | 1.71 | 0.00 | 0.00 | 0.38 | 0.85 | 0.00 | 0.00 | 36.88 | 54.75 | 0.00 | 0.00 | 5.46 | 0.00 | 0.00 |
| 160 | 0.00 | 0.00 | 0.00 | 0.00 | 1.50 | 6.50 | 0.00 | 3.00 | 64.56 | 21.94 | 2.50 | 0.00 | 0.00 | 0.00 | 0.00 |
| 161 | 0.00 | 0.00 | 0.00 | 0.00 | 2.77 | 0.00 | 0.00 | 3.00 | 73.30 | 18.42 | 2.50 | 0.00 | 0.00 | 0.00 | 0.00 |
| 162 | 0.00 | 0.00 | 0.00 | 0.00 | 4.00 | 3.67 | 0.00 | 3.00 | 61.81 | 25.02 | 2.50 | 0.00 | 0.00 | 0.00 | 0.00 |
| 163 | 0.00 | 0.00 | 0.00 | 0.00 | 1.50 | 3.12 | 0.00 | 3.00 | 62.47 | 27.41 | 2.50 | 0.00 | 0.00 | 0.00 | 0.00 |
| 164 | 0.00 | 0.00 | 0.00 | 0.00 | 3.12 | 4.75 | 0.00 | 3.00 | 77.66 | 8.97 | 2.50 | 0.00 | 0.00 | 0.00 | 0.00 |
| 165 | 0.00 | 0.00 | 0.00 | 0.00 | 3.08 | 4.25 | 0.00 | 3.00 | 62.80 | 26.87 | 0.00 | 0.00 | 0.00 | 0.00 | 0.00 |
| 166 | 0.00 | 0.00 | 0.00 | 0.00 | 4.00 | 0.00 | 0.00 | 3.00 | 72.40 | 18.10 | 2.50 | 0.00 | 0.00 | 0.00 | 0.00 |
| 167 | 0.00 | 0.00 | 0.00 | 0.00 | 2.16 | 0.19 | 0.00 | 3.00 | 67.15 | 25.23 | 2.26 | 0.00 | 0.00 | 0.00 | 0.00 |
| 168 | 0.00 | 0.00 | 0.00 | 0.00 | 0.00 | 3.50 | 0.00 | 2.98 | 50.30 | 41.63 | 1.99 | 0.00 | 0.00 | 0.00 | 0.00 |
| 169 | 0.00 | 0.00 | 2.69 | 0.00 | 2.68 | 2.67 | 0.00 | 2.92 | 49.94 | 37.17 | 1.98 | 0.00 | 0.00 | 0.00 | 0.00 |
| 170 | 0.00 | 0.00 | 3.50 | 0.00 | 3.50 | 0.00 | 0.00 | 3.03 | 50.03 | 38.05 | 2.00 | 0.00 | 0.00 | 0.00 | 0.00 |
| 171 | 0.00 | 0.00 | 1.82 | 0.00 | 1.66 | 1.72 | 0.00 | 2.98 | 38.21 | 52.72 | 1.03 | 0.00 | 0.00 | 0.00 | 0.00 |
| 172 | 0.00 | 0.00 | 1.02 | 0.00 | 1.01 | 0.97 | 0.00 | 3.03 | 40.98 | 53.39 | 1.00 | 0.00 | 0.00 | 1.02 | 0.00 |
| 173 | 0.00 | 1.80 | 0.00 | 0.00 | 0.40 | 0.88 | 0.00 | 0.00 | 38.96 | 57.75 | 0.00 | 0.00 | 0.00 | 0.00 | 0.00 |
| 174 | 0.00 | 0.00 | 0.00 | 0.00 | 0.38 | 0.88 | 1.80 | 0.00 | 37.32 | 55.46 | 0.00 | 0.00 | 0.00 | 0.00 | 0.00 |
| 175 | 0.30 | 7.92 | 0.00 | 0.00 | 1.78 | 0.00 | 0.00 | 0.00 | 33.03 | 56.97 | 0.00 | 0.00 | 0.00 | 0.00 | 0.00 |
| 176 | 0.30 | 7.95 | 0.00 | 0.00 | 1.78 | 0.00 | 0.00 | 0.00 | 32.99 | 56.98 | 0.00 | 0.00 | 0.00 | 0.00 | 0.00 |
| 177 | 0.30 | 7.87 | 0.00 | 0.00 | 1.78 | 0.00 | 0.00 | 0.00 | 33.00 | 56.93 | 0.00 | 0.00 | 0.00 | 0.00 | 0.12 |
| 178 | 0.30 | 7.89 | 0.00 | 0.00 | 1.77 | 0.00 | 0.00 | 0.00 | 32.93 | 57.10 | 0.00 | 0.00 | 0.00 | 0.00 | 0.00 |
| 179 | 0.30 | 7.81 | 0.00 | 0.00 | 1.75 | 0.00 | 0.00 | 0.00 | 32.59 | 56.51 | 0.00 | 0.00 | 0.00 | 1.01 | 0.03 |
| 180 | 0.40 | 7.88 | 0.00 | 0.00 | 1.77 | 0.00 | 0.00 | 0.00 | 32.89 | 57.03 | 0.00 | 0.00 | 0.00 | 1.01 | 0.03 |
| 181 | 0.70 | 0.60 | 0.00 | 0.00 | 0.41 | 0.00 | 0.00 | 0.00 | 38.94 | 57.82 | 0.00 | 1.41 | 0.00 | 0.00 | 0.13 |
| 182 | 0.90 | 1.84 | 0.00 | 0.00 | 0.40 | 0.00 | 0.00 | 0.00 | 38.98 | 57.88 | 0.00 | 0.00 | 0.00 | 0.00 | 0.00 |
| 183 | 0.89 | 1.78 | 0.00 | 0.00 | 0.40 | 0.00 | 0.00 | 0.00 | 38.60 | 57.31 | 0.00 | 0.00 | 0.00 | 1.00 | 0.03 |
| 184 | 0.00 | 0.00 | 0.00 | 1.53 | 0.64 | 0.90 | 0.00 | 2.43 | 39.21 | 52.97 | 1.26 | 0.00 | 0.00 | 1.00 | 0.05 |
| 185 | 0.00 | 0.00 | 0.00 | 1.52 | 0.63 | 0.90 | 0.00 | 2.52 | 39.23 | 52.93 | 1.24 | 0.00 | 0.00 | 1.00 | 0.03 |

*pH adjuster used,
Q.S. = quantum satis,
pH was adjusted using NaOH or HCl

TABLE 4
Properties of Exemplary Compositions Appearing in Table 3

| | Mean Viscosity | | | |
|---|---|---|---|---|
| # | (mPas) | Sprayability | Mucoadhesion | pH |
| 120 | 1642.33 | Yes | Yes | 2.66 |
| 121 | 1483.33 | No | Yes | 2.61 |
| 122 | 524.00 | No | Yes | 2.74 |
| 123 | 17390.00 | No | Yes | 2.67 |
| 124 | 2050.00 | No | Yes | 2.42 |
| 125 | 576.67 | No | Yes | 2.52 |
| 126 | 519.00 | Yes | Yes | 2.56 |
| 127 | 464.00 | Yes | Yes | 2.62 |
| 128 | 481.33 | Yes | Yes | 2.56 |
| 129 | 488.33 | Yes | Yes | 2.54 |
| 130 | 1000.67 | Yes | Yes | 2.53 |
| 131 | 1315.33 | No | Yes | 2.63 |
| 132 | 98.67 | Yes | Yes | 2.53 |
| 133 | 370.33 | Yes | Yes | 2.55 |
| 134 | 480.00 | Yes | Yes | 2.56 |
| 135 | 457.67 | Yes | Yes | 2.50 |
| 136 | 329.67 | Yes | Yes | 2.56 |
| 137 | 297.67 | Yes | Yes | 2.51 |
| 138 | 2180.00 | No | Yes | 2.55 |
| 139 | 896.67 | Yes | Yes | 6.50 |
| 140 | 1980.00 | No | Yes | 2.58 |
| 141 | 630.00 | Yes | Yes | 5.50 |
| 142 | 1172.67 | Yes | Yes | 2.74 |
| 143 | 1426.33 | Yes | Yes | 5.53 |
| 144 | 1114.33 | No | Yes | 6.79 |
| 145 | 1115.00 | No | Yes | 2.59 |
| 146 | 1052.00 | No | Yes | 6.23 |
| 147 | 1198.00 | No | Yes | 2.94 |
| 148 | 1328.00 | No | Yes | 2.74 |
| 149 | 1155.00 | No | Yes | 4.40 |
| 150 | 1070.67 | Yes | Yes | 2.62 |
| 151 | 1090.67 | No | Yes | 5.12 |
| 152 | 748.67 | Yes | Yes | 5.90 |
| 153 | 570.00 | Yes | Yes | 2.77 |
| 154 | 1122.33 | No | Yes | 2.65 |
| 155 | 455.67 | Yes | Yes | 6.26 |
| 156 | 1010.33 | Yes | Yes | 2.54 |
| 157 | 276.67 | Yes | Yes | 2.58 |
| 158 | 419.00 | Yes | Yes | 5.98 |
| 159 | 397.33 | Yes | Yes | 5.88 |
| 160 | NT | No | Yes | 2.80 |
| 161 | NT | Yes | Yes | 2.70 |
| 162 | NT | No | Yes | 2.70 |
| 163 | NT | Yes | Yes | 2.70 |
| 164 | NT | No | Yes | 3.10 |
| 165 | NT | No | Yes | 7.20 |
| 166 | NT | No | Yes | 2.90 |
| 167 | NT | Yes | Yes | 2.95 |
| 168 | NT | Yes | Yes | 2.50 |
| 169 | NT | No | Yes | 3.00 |
| 170 | NT | No | Yes | 3.00 |
| 171 | NT | Yes | Yes | 3.00 |
| 172 | NT | Yes | Yes | 3.00 |
| 173 | 249.00 | Yes | Yes | 4.93 |
| 174 | 303.00 | Yes | Yes | 4.53 |
| 175 | NT | Yes | NT | NT |
| 176 | NT | Yes | NT | NT |
| 177 | NT | Yes | NT | NT |
| 178 | NT | Yes | NT | NT |
| 179 | 212.33 | Yes | Yes | 4.77 |
| 180 | 351.33 | Yes | Yes | 4.69 |
| 181 | NT | Yes | NT | NT |
| 182 | NT | Yes | Yes | 6.79 |
| 183 | NT | Yes | Yes | NT |
| 184 | NT | Yes | Yes | 2.65 |
| 185 | NT | Yes | Yes | NT |

NT = Not tested

TABLE 5
Exemplary compositions 5.1 to 5.4

| | Concentration by weight (%) | | | |
|---|---|---|---|---|
| | Example | Example | Example | Example |
| Excipient | 5.1 | 5.2 | 5.3 | 5.4 |
| Carbopol | 0.2-3 | 0.2-0.9 | 0.2-0.9 | 0.3 |
| PEG400 | 0-76 | 0.75-76 | 1.5-76 | 7.92 |
| Ethanol | 0-40 | 12-40 | 12-35 | 33 |
| Water | 0.5-63 | 10.5-63 | 10.5-58 | 57 |
| Cellulose derivative (HPC) | 0-1.8 | 0-1.8 | 0-1.8 | — |
| Cellulose derivative (MC) | 0-3 | 0-1.8 | 0.03-1.8 | 1.78 |
| Cellulose derivative (total) | 0-3 | 0-1.8 | 0.3-1.8 | — |
| Glycerol | 0-5 | 0-3 | 0.9-3 | — |
| Cyclodextrin | 0-30 | 0-30 | — | — |

TABLE 6
Exemplary compositions 6.1 to 6.4

| | Concentration by weight (%) | | | |
|---|---|---|---|---|
| | Example | Example | Example | Example |
| Excipient | 6.1 | 6.2 | 6.3 | 6.4 |
| PVP-VA-64 | 0.7-10 | 5-10 | 7-10 | 10 |
| PEG400 | 1.85-77 | 1.85-77 | 40-65 | 61.8 |
| Ethanol | 4.3-40 | 4.4-39 | 15-20 | 14 |
| Water | 8-54 | 8-54 | 10-40 | 10 |
| Citric Acid monohydrated | 0-1.7 | 0-0.12 | 0-0.12 | 0.12 |
| Cellulose derivative | 0-1.5 | 0.4-1.5 | 1-1.5 | — |
| (HPC (individual or mixture)) | | | | |
| Glycerol | 0-3 | 0-3 | 1.5-3 | 1.5 |
| Flavor (Mint's and derivate) | 0-2.5 | 0-2.5 | 0-2.5 | 2.5 |
| Sweetener (Sucralose) | 0-0.03 | 0-0.03 | 0.01-0.03 | 0.03 |

TABLE 7
Exemplary compositions 7.1 to 7.5

| | Concentration by weight (%) | | | | |
|---|---|---|---|---|---|
| | Example | Example | Example | Example | Example |
| Excipient | 7.1 | 7.2 | 7.3 | 7.4 | 7.5 |
| HPC-M | 0.3-4 | 0.3-2.7 | 0.3-1.8 | 0.4 | 0.6 |
| HPC-G | 0.19-6.5 | 0.6-3.5 | 0.6-1.8 | 0.9 | 0.9 |
| Total HPCs (M + G) | 0.4-8 | 0.4-4.6 | 0.4-3.6 | 1.3 | 1.3 |
| PVP-VA-64 | 0-2 | 0-2 | 0-2 | — | — |
| PVP-30 | 0-3.5 | 0-1.8 | — | — | — |
| PEG400 | 0.6-7.9 | 0-1.8 | 0.6-1.8 | 1.8 | 1.8 |
| Ethanol | 15-77 | 33-40 | 36-40 | 39 | 39 |
| Water | 8.9-59.5 | 50-58 | 51-58 | 57.9 | 56 |
| Additional HPCs | 0-1.84 | 0-1.84 | 0-1.84 | — | — |
| Cellulose derivative (MC) | 0-1.8 | 0-1.8 | 0-1.8 | — | — |
| Salicylic acid | 0-2.5 | 0-2.5 | — | — | — |
| Citric acid | 0-1.7 | 0.1-1.7 | 0.1-1.7 | — | 1.7 |
| Glycerol | 0-3.5 | 0-3.5 | 1.5-3.5 | — | — |
| Cyclodextrin | 0-5.5 | 0-5.5 | — | — | — |
| Carbopol | 0-0.9 | 0-0.9 | — | — | — |

### EQUIVALENTS AND SCOPE

In the claims articles such as “a,” “an,” and “the” may mean one or more than one unless indicated to the contrary or otherwise evident from the context. For example, “a cellulose derivative” refers to “one or more cellulose derivatives”; and “the concentration of the cellulose derivative” refers to “the combined concentration of the one or more cellulose derivatives.” Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising,” “including,” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A composition comprising:
(i) a polyacrylic acid, wherein the concentration of the polyacrylic acid in the composition is between 0.1% and 10%, inclusive, by weight;
(ii) a polyethylene glycol (PEG), wherein the concentration of the PEG in the composition is between 0% and 90%, inclusive, by weight;
(iii) ethanol, a propanol, a butanol, or a mixture thereof, wherein the concentration of component (iii) in the composition is between 0% and 60%, inclusive, by weight; and
(iv) water, wherein the concentration of water in the composition is between 10% and 75%, inclusive, by weight;
provided that the total concentration of all components of the composition is 100%, inclusive, by weight.

2. The composition of claim 1, wherein the polyacrylic acid is a Carbopol® polymer.

3-28. (canceled)

29. The composition of claim 1, wherein the composition is as described in Tables 1A, 1B, and 5.

30. A composition comprising:
(i) a polyvinylpyrrolidone copolymer, wherein the concentration of the polyvinylpyrrolidone copolymer in the composition is between 0.1% and 25%, inclusive, by weight;
(ii) a polyethylene glycol (PEG), wherein the concentration of the PEG in the composition is between 1% and 90%, inclusive, by weight;
(iii) ethanol, a propanol, a butanol, or a mixture thereof, wherein the concentration of component (iii) in the composition is between 1% and 50%, inclusive, by weight; and
(iv) water, wherein the concentration of water in the composition is between 5% and 75%, inclusive, by weight;
provided that the total concentration of all components of the composition is 100%, inclusive, by weight.

31-56. (canceled)

57. A composition comprising:
(i) one or both of a hydroxypropyl cellulose M and a hydroxypropyl cellulose G, wherein:
when the composition comprises a hydroxypropyl cellulose M, the concentration of the hydroxypropyl cellulose M in the composition is between 0.1% and 5%, inclusive, by weight;
when the composition comprises a hydroxypropyl cellulose G, the concentration of the hydroxypropyl cellulose G in the composition is between 0.1% and 7%, inclusive, by weight; and
when the composition comprises only one or both of a hydroxypropyl cellulose M and a hydroxypropyl cellulose G, the composition further comprises a polyvinylpyrrolidone copolymer, wherein the concentration of the polyvinylpyrrolidone copolymer in the composition is between 0% and 10%, inclusive, by weight;
(ii) ethanol, a propanol, a butanol, or a mixture thereof, wherein the concentration of component (ii) thereof in the composition is between 10% and 80%, inclusive, by weight; and
(iii) water, wherein the concentration of water in the composition is between 8% and 75%, inclusive, by weight;
provided that the total concentration of all components of the composition is 100%, inclusive, by weight.

58-91. (canceled)

92. The composition of claim 1 further comprising an active agent, wherein the concentration of the active agent in the composition is between 0.1% and 50%, inclusive, by weight.

93-96. (canceled)

97. A kit comprising:
a composition of claim 1; and
instructions for using the composition.

98. A method of delivering an active agent to a subject in need thereof comprising contacting a mucus of the subject in need thereof with a composition of claim 92.

99. A method of treating a disease in a subject in need thereof comprising contacting a mucus of the subject in need thereof with an effective amount of a composition of claim 92.

100. A method of preventing a disease in a subject in need thereof comprising contacting a mucus of the subject in need thereof with an effective amount of a composition of claim 92.

101-103. (canceled)

104. The composition of claim 30, further comprising an active agent, wherein the concentration of the active agent in the composition is between 0.1% and 50%, inclusive, by weight.

105. A kit comprising:
a composition of claim 30; and
instructions for using the composition.

106. A method of delivering an active agent to a subject in need thereof comprising contacting a mucus of the subject in need thereof with a composition of claim 104.

107. A method of treating a disease in a subject in need thereof comprising contacting a mucus of the subject in need thereof with an effective amount of a composition of claim 104.

108. A method of preventing a disease in a subject in need thereof comprising contacting a mucus of the subject in need thereof with an effective amount of a composition of claim 104.

109. The composition of claim 57, further comprising an active agent, wherein the concentration of the active agent in the composition is between 0.1% and 50%, inclusive, by weight.

110. A kit comprising:
a composition of claim 57; and
instructions for using the composition.

111. A method of delivering an active agent to a subject in need thereof comprising contacting a mucus of the subject in need thereof with a composition of claim 109.

112. A method of treating a disease in a subject in need thereof comprising contacting a mucus of the subject in need thereof with an effective amount of a composition of claim 109.

113. A method of preventing a disease in a subject in need thereof comprising contacting a mucus of the subject in need thereof with an effective amount of a composition of claim 109.
